(19) United States
(12) Patent Application Publication
(10) Pub. No.: US 2012/0014970 A1
(43) Pub. Date: Jan. 19, 2012

(54) Therapeutic Compositions for Treatment of Corneal Disorders

(76) Inventors: Reza Dana, Cambridge, MA (US); Mohammad Dastjerdi, Prairie Village, KS (US); Sunil Chauhan, Somerville, MA (US)

(21) Appl. No.: 13/183,231

(22) Filed: Jul. 14, 2011

Related U.S. Application Data
(63) Continuation-in-part of application No. 12/685,510, filed on Jan. 11, 2010.
(60) Provisional application No. 61/143,561, filed on Jan. 9, 2009.

(51) Int. Cl. A61K 39/395; A61P 29/00; A61P 25/00; A61K 38/16; A61P 27/02

(52) U.S. Cl. 424/158.1; 514/7.6; 424/172.1

(57) ABSTRACT

The invention provides methods and compositions for minimizing, preventing, or treating damage to corneal nerves by administering to a subject with such damage or at risk of exposure to such damage a composition which blocks an activity of an IL-1 cytokine and/or an IL-17 cytokine.

### RELATED APPLICATIONS

This application is a continuation-in-part of and claims the benefit of priority of U.S. Ser. No. 12/685,510, filed Jan. 11, 2010, and U.S. Ser. No. 61/143,561, filed Jan. 9, 2009, the contents of each of which are incorporated by reference herein in their entirety.

### FIELD OF THE INVENTION

This invention relates generally to the field of ophthalmology.

### BACKGROUND OF THE INVENTION

Corneal epithelial damage can lead to chronic ocular surface disease. The mechanisms by which this occurs have not been elucidated, making the development of treatments that address the cause rather than the symptoms of chronic ocular surface disease difficult, if not impossible. As such, there has been a long-felt need in the art for the discovery of these mechanisms and for the development of compositions and methods of treatment.

### SUMMARY OF THE INVENTION

The invention is based on the surprising discovery that IL-1 inhibition leads to corneal nerve regeneration. Moreover, the invention provides compositions and methods for treating neurotrophic dry eye disease by reducing damage to and regenerating corneal nerves. Nerve damage and increased immune activity within the cornea complete a vicious cycle of events, along with corneal epithelial damage, that would perpetuate itself and lead to chronic ocular surface disorders, but for the intervention of the treatments described herein. Neurotrophic dry eye (a neuropathic condition) is distinguished from other types of dry eye by a reduction or loss of corneal nerve tissue. For example, neurotrophic dry eye is characterized by a reduction or loss of at least about 10%, 25%, 50%, 75%, or more of corneal nerve tissue or corneal nerve fiber length compared to a normal condition.

The invention provides a method for protecting or treating corneal nerves in a subject in need thereof, including the steps of: (a) identifying a subject with corneal nerve damage or loss; and (b) locally administering to the cornea of the subject a composition that inhibits an activity of an inflammatory cytokine (e.g., IL-1 or a combination of IL-1 and IL-17), thereby enhancing corneal nerve regeneration and reducing the development of abnormalities in nerve morphology or density. Preferably, the subject has not been diagnosed as having meibomian gland dysfunction (MGD), e.g., posterior blepharitis.

In one aspect of the above method, the subject is identified as having corneal nerve damage or loss that results from a congenital defect, disease, trauma, medical or surgical procedure. In another aspect of the above method, the subject is identified as having corneal nerve damage or loss that results from neurotrophic keratitis, herpes simplex, zoster keratitis, diabetes mellitus, trigeminal nerve damage, orbital or head surgery, head trauma, aneurysm, intracranial neurologic disease, keratorefractive procedures, photorefractive keratectomy (PRK), laser in situ keratomileusis (LASIK), congenital defect, ocular surface disease, dry eye syndrome, a non-ophthalmic disorder, a non-ophthalmic procedure, peripheral neuropathy, or diabetic neuropathy.

The invention further provides a method for minimizing or preventing damage or loss of corneal nerves in a subject in need thereof, including the steps of: (a) identifying the subject at risk of developing corneal nerve damage or loss; and (b) locally administering to the cornea of the subject a composition that inhibits an activity of an inflammatory interleukin-1 or interleukin-17 cytokine prior to development of nerve damage or loss, thereby decreasing nerve degeneration and reducing or preventing the development of abnormalities in nerve morphology or density.

In one aspect of the above method, the subject is identified as being at risk of exposure to corneal nerve damage or loss that could result from disease, trauma, or a medical procedure. In another aspect of the above method, the subject is identified as being at risk of exposure to corneal nerve damage or loss that could result from neurotrophic keratitis, herpes simplex keratitis, herpes zoster keratitis, diabetes mellitus, trigeminal nerve damage, orbital or head surgery, head trauma, aneurysm, intracranial neurologic disease, keratorefractive procedures, photorefractive keratectomy (PRK), laser in situ keratomileusis (LASIK), ocular surface disease, dry eye syndrome, a non-ophthalmic disorder, a non-ophthalmic procedure, peripheral neuropathy, or diabetic neuropathy.

In certain embodiments, the above methods further include the step of identifying a subject with a sign or symptom of corneal nerve damage or loss. For example, a sign of corneal nerve damage or loss is a decrease of corneal innervation or sensation, a reduction in the number of nerve fibers or bundles innervating the cornea, death of neurons innervating the cornea, a decrease or loss of neurotransmitter release, a decrease or loss of nerve growth factor release, abnormal tearing reflexes, abnormal blink reflexes, abnormal nerve morphology, appearance of abnormal nerve sprouts, abnormal tortuosity, increased bead-like nerve formations, thinning of nerve fiber bundles, or thickening of nerve fiber bundles. For example, a symptom of corneal nerve damage or loss is abnormal tear production or dryness, abnormal blinking, and difficulty or loss of ability to focus, decreased or lost visual acuity, or decreased or lost corneal sensitivity.

In one aspect of the above methods, the activity includes binding of an inflammatory IL-1 cytokine to an IL-1 receptor. Compositions of the above methods that inhibit binding of an inflammatory IL-1 cytokine to an IL-1 receptor include an amino acid sequence of SEQ ID NO: 16. Compositions optionally include inhibitors of IL-17 activity, e.g., compounds that inhibit IL-17 binding to its receptor, or compounds that inhibit cytokines critical for generation of T helper-17/IL-17 response, such as inhibitors of IL-6 or inhibitors of IL-23. Preferably, the compositions do not include generic, broad spectrum immunosuppressive agents, such as cyclosporine A (CsA), as such non-specific suppressors of inflammation do not regenerate corneal nerves.

In each of the methods described herein, the composition is present in a concentration of 0.1-10% (weight/volume or w/v). Alternatively, the composition is present in a concentration of 1.0% (mg/ml), 1.5% (mg/ml), 2.0% (mg/ml), 2.5% (mg/ml), 3.0% (mg/ml), 3.5% (mg/ml), 4.0% (mg/ml), 4.5% (mg/ml), 5.0% (mg/ml), 5.5% (mg/ml), 6.0% (mg/ml), 6.5% (mg/ml), 7.0% (mg/ml), 7.5% (mg/ml), 8.0% (mg/ml), 8.5% (mg/ml), 9.0% (mg/ml), 9.5% (mg/ml), 10.0% (mg/ml), or any percentage point in between. In a preferred embodiment, the composition is present in a concentration of 2.5% (mg/ml) or 5% (mg/ml). For example, the composition is present in a concentration of 25 mg/ml or 50 mg/ml. Exemplary formulations contain an inhibitory composition present in a concentration of 2.5% (25 mg/ml) or 5% (50 mg/ml).

The form of a composition of the above methods is a solid, a paste, an ointment, a gel, a liquid, an aerosol, a mist, a polymer, a film, an emulsion, or a suspension. The composition is administered topically. In a preferred embodiment, the above methods do not include systemic administration or substantial dissemination to non-ocular tissue. In certain embodiments of the above methods, the composition further includes a compound selected from the group consisting of a physiological acceptable salt, poloxamer analogs with carbopol, carbopol/HPMC, carbopol-methyl cellulose, a mucolytic agent, carboxymethylcellulose (CMC), hyaluronic acid, cyclodextrin, and petroleum. An exemplary mucolytic agent is N-acetyl cysteine. In a preferred embodiment, the composition further includes carboxymethylcellulose (CMC).

Compositions of the above methods inhibit the transcription, transcript stability, translation, modification, localization, secretion, or function of a polynucleotide or polypeptide encoding an inflammatory interleukin-1 cytokine or an IL-1 receptor. In certain embodiments, a composition of the above methods includes a polynucleotide, a polypeptide, an antibody, or a small molecule. Alternatively, or in addition, a composition of the above methods includes a morpholino antisense oligonucleotide, microRNA (miRNA), short hairpin RNA (shRNA), or short interfering RNA (siRNA).

The invention provides a method for reducing or treating corneal lymphangiogenesis in a subject in need thereof, including the steps of: (a) identifying a subject with corneal lymphangiogenesis; and (b) locally administering to the cornea of the subject a composition that inhibits an activity of an inflammatory interleukin-1 cytokine, thereby inhibiting the ability of lymphatic vessels to expand within or invade corneal tissue and reducing or treating corneal lymphangiogenesis.

The invention provides a method for minimizing or preventing corneal lymphangiogenesis in a subject in need thereof, including the steps of: (a) identifying a subject at risk of developing lymphangiogenesis onset; and (b) locally administering to the cornea of the subject a composition that inhibits an activity of an inflammatory interleukin-1 cytokine prior to the development, thereby inhibiting the ability of lymphatic vessels to form or expand within, or to invade corneal tissue and minimizing or preventing corneal lymphangiogenesis.

The invention provides a method for reducing or treating the induction of immunity in a cornea of a subject in need thereof, including the steps of: (a) identifying a subject with an induction of immunity; and (b) locally administering to the cornea of the subject a composition that inhibits an activity of an inflammatory interleukin-1 cytokine, thereby inhibiting the ability of lymphatic vessels to expand within or to invade corneal tissue and reducing or treating the induction of immunity, wherein the lymphatic vessels permit the transport of immune cells between the corneal tissue and lymph nodes and the initiation of an immune response.

The invention provides a method for minimizing or preventing induction of immunity in a cornea of a subject in need thereof, including the steps of: (a) identifying the subject at risk of developing an immunity; and (b) locally administering to the cornea of the subject a composition that inhibits an activity of an inflammatory interleukin-1 cytokine prior to the development, thereby inhibiting the ability of lymphatic vessels to expand within or to invade corneal tissue and minimizing or preventing induction of immunity, wherein the lymphatic vessels permit the transport of immune cells between the corneal tissue and lymph nodes and the initiation of an immune response.

The invention provides a method for reducing or treating an autoimmune condition affecting a corneal tissue of a subject in need thereof, including the steps of: (a) identifying a subject with the autoimmune condition; and (b) locally administering to the cornea of the subject a composition that inhibits an activity of an inflammatory interleukin-1 cytokine, thereby inhibiting the ability of lymphatic vessels to expand within or to invade corneal tissue and reducing or treating the autoimmune condition, wherein the lymphatic vessels permit the transport of immune cells between the corneal tissue and lymph nodes and the initiation of an immune response.

The invention provides a method for minimizing or preventing the development of an autoimmune condition affecting a corneal tissue of a subject in need thereof, including the steps of: (a) identifying a subject at risk of developing said autoimmune condition; and (b) locally administering to the cornea of the subject a composition that inhibits an activity of an inflammatory interleukin-1 cytokine prior to the development, thereby inhibiting the ability of lymphatic vessels to expand within or to invade corneal tissue and minimizing or preventing the development of the autoimmune condition, wherein the lymphatic vessels permit the transport of immune cells between said corneal tissue and lymph nodes and the initiation of an immune response.

Also provided is a method of reducing ocular pain in a subject in need thereof. The method includes administering, e.g., topically, to a subject experiencing ocular pain, an inhibitor of an activity of an inflammatory interleukin-1 cytokine, e.g., a protein that inhibits binding of an inflammatory IL-1 cytokine to an IL-1 receptor, in an amount effective reduce ocular pain. The protein can be, for example, a protein inhibitor of inflammatory interleukin-1 cytokines described herein, such as a protein including an amino acid sequence of SEQ ID NO: 16 or an antibody that binds to IL-1α, IL-1β or IL-1RI. In certain embodiments, the inhibitor is delivered in an eye drop. The ocular pain can be pain mediated by corneal nerves or pain involving sensation at or around the ocular surface, e.g., pain involving the cornea or conjunctiva.

In certain embodiments of the above methods, the subject has a dry-eye associated ocular surface disease. Alternatively, or in addition, the subject is at risk of developing a dry-eye associated ocular surface disease. In some embodiments, the subject has an inflammatory surface of the eye disease, e.g., in addition to experiencing ocular pain. In some embodiments, the subject does not have an inflammatory surface of the eye disease, or the subject does not have a dry-eye associated ocular surface disease, or risk of developing such disease. In some embodiments, the subject does not have allergic conjunctivitis, e.g., severe allergic conjunctivitis.

In some embodiments, the subject has normal intraocular pressure; in some embodiments, the subject has abnormal intraocular pressure. In some embodiments, the subject is a subject who does not have glaucoma, a retinal nerve disease or disorder, neurodegenerative diseases of the central nervous systems, a disease or disorder of the optic nerve or the retina, optic neuritis, macular or retinal degeneration, retinitis pigmentosa, or diabetic retinopathy.

The ability of lymphatic vessels to expand within or to invade corneal tissue encompasses the potential or actual growth, expansion, elaboration, splitting, or remodeling of lymphatic vessels either within a corneal tissue or from a non-corneal tissue (such as the adjacent limbus) into corneal tissue. The phrase “lymphatic vessels permit the transport of immune cells” describes the unidirectional or bidirectional movement or deposition of an immune cell between a corneal tissue and a non-corneal tissue, preferably, a lymph node or other sites in the lymphoid compartment. Exemplary immune cells is include, but are not limited to, T cells, B cells, dendritic cells, macrophages, monocytes, and natural killer (NK) cells.

In one aspect of the above methods, the activity of an inflammatory interleukin-1 cytokine includes binding of an inflammatory IL-1 cytokine to an IL-1 receptor. Compositions of the above methods that inhibit binding of an inflammatory IL-1 cytokine to an IL-1 receptor include an amino acid sequence of SEQ ID NO: 16.

The form of a composition of the above methods is a solid, a paste, an ointment, a gel, a liquid, an aerosol, a mist, a polymer, a film, an emulsion, or a suspension. The composition is administered topically. In a preferred embodiment, the above methods do not include systemic administration or substantial dissemination to non-ocular tissue. In certain embodiments of the above methods, the composition further includes a compound selected from the group consisting of a physiological acceptable salt, poloxamer analogs with carbopol, carbopol/HPMC, carbopol-methyl cellulose, a mucolytic agent, carboxymethylcellulose (CMC), hyaluronic acid, cyclodextrin, and petroleum. An exemplary mucolytic agent is N-acetyl cysteine. In a preferred embodiment, the composition further includes carboxymethylcellulose (CMC).

Compositions of the above methods inhibit the transcription, transcript stability, translation, modification, localization, secretion, or function of a polynucleotide or polypeptide encoding an inflammatory interleukin-1 cytokine or an IL-1 receptor. In certain embodiments, a composition of the above methods includes a polynucleotide, a polypeptide, an antibody, or a small molecule. Alternatively, or in addition, a composition of the above methods includes a morpholino antisense oligonucleotide, microRNA (miRNA), short hairpin RNA (shRNA), or short interfering RNA (siRNA).

In certain embodiments of the above methods, the activity includes binding of an inflammatory IL-1 cytokine to an IL-1 receptor. Furthermore, compositions of the above methods that inhibit binding of an inflammatory IL-1 cytokine to an IL-1 receptor include the amino acid sequence of SEQ ID NO: 16. In certain embodiments, compositions of the above methods are present in a concentration of 0.1-10% (mg/ml). Alternatively, the composition is present in a concentration of 1.0% (mg/ml), 1.5% (mg/ml), 2.0% (mg/ml), 2.5% (mg/ml), 3.0% (mg/ml), 3.5% (mg/ml), 4.0% (mg/ml), 4.5% (mg/ml), 5.0% (mg/ml), 5.5% (mg/ml), 6.0% (mg/ml), 6.5% (mg/ml), 7.0% (mg/ml), 7.5% (mg/ml), 8.0% (mg/ml), 8.5% (mg/ml), 9.0% (mg/ml), 9.5% (mg/ml), 10.0% (mg/ml), or any percentage point in between. In a preferred embodiment, the composition is present in a concentration of 2.5% (mg/ml) or 5% (mg/ml). In another preferred embodiment, the composition is present in a concentration of 25 mg/ml or 50 mg/ml. In a further preferred embodiment, the composition is present in a concentration of 2.5% (25 mg/ml) or 5% (50 mg/ml).

In one aspect of the invention, the form of the compositions of the above methods is a solid, a paste, an ointment, a gel, a liquid, an aerosol, a mist, a polymer, a film, an emulsion, or a suspension.

Compositions of the above methods are administered topically. The above methods do not include systemic administration or substantial dissemination of the composition to non-ocular tissue.

In certain embodiments, compositions of the above methods further include a compound selected from the group consisting of a physiological acceptable salt, poloxamer analogs with carbopol, carbopol/HPMC, carbopol-methyl cellulose, N-acetyl cysteine, carboxymethylcellulose (CMC), hyaluronic acid, cyclodextrin, and petroleum. Preferably, the composition further includes N-acetyl cysteine or carboxymethylcellulose (CMC).

Alternatively, compositions of the above methods inhibit or enhance the transcription, transcript stability, translation, modification, localization, secretion, or function of a polynucleotide or polypeptide encoding the IL-1 receptor, type 2 (IL-1R2). IL-1R2 binds IL-1 and can inhibit the function of IL-1R1. Thus, in one embodiment, enhancement of IL-1R2 function provides another mechanism by which IL-1R1 activity is inhibited. In this same embodiment, inhibition of an antagonist of IL-1R2, specifically, IL-1Ra3, inhibits IL-1R1 function. Thus, the composition alone, or in combination with an enhancer of IL-1R2, inhibits the transcription, transcript stability, translation, modification, localization, secretion, or function of a polynucleotide or polypeptide encoding IL-1Ra3, SEQ ID NO: 22 or 23. Alternatively, in an embodiment wherein IL-1R2 receptor function augments the activity of IL-1R1, the composition contains one or more regions of a polynucleotide or polypeptide encoding IL-1Ra3 to augment IL-1R2 inhibition. Furthermore, the composition of this embodiment comprises the whole polynucleotide or polypeptide encoding IL-1Ra3.

Compositions of the methods of the invention include a polynucleotide, a polypeptide, an antibody, a compound, or a small molecule with means to inhibit the transcription, transcript stability, translation, modification, localization, secretion, or function of a polynucleotide or polypeptide encoding an accessory protein of an IL-1 Receptor. For example, this IL-1 receptor accessory protein is IL-1RAP, which directly binds IL-1 and IL-1R1, and is defined by the polynucleotide sequence of SEQ ID NO: 24 or 26 and the polypeptide sequence of SEQ ID NO: 25 or 27. IL-1RAP belongs to a signaling complex that is required for signal transduction from IL-1R1. Thus, inhibition of IL-1RAP antagonizes IL-1R1 function.

In another embodiment, compositions of the methods of the invention include a polynucleotide, a polypeptide, an antibody, a compound, or a small molecule with means to inhibit the transcription, transcript stability, translation, modification, localization, secretion, or function of a polynucleotide or polypeptide encoding an associated kinase to an IL-1 receptor. For example, IL-1 receptor-associated kinase is IRAK1. IRAK1 is a downstream signaling effector that leads to transcriptional events associated with escalating inflammatory responses and is defined by the polynucleotide sequence of SEQ ID NO: 28, 30, or 32 and the polypeptide sequence of SEQ ID NO: 29, 31, or 33. Upon IL-1 receptor binding by IL-1, IRAK1 is recruited to the receptor complex, becomes hyperphosphorylated, and participates in the formation of a new protein complex consisting of hyperphosphorylated IRAK1 and TRAF6. The formation of this IRAK1/TRAF6 complex is a prerequisite for tumor necrosis factor (TNF) associated factor 6 (TRAF6)-mediated activation of nuclear factor-κB (NF-κB). Thus, the modification of the expression or function of any component of the above-delineated signaling cascade indicates a binding event between IL-1 to an IL-1 receptor.

Compositions of the methods of the invention include a polynucleotide, a polypeptide, an antibody, or a small molecule that binds or modifies the function of IL-1α, IL-1β, IL-1R1, IL-1R2, IL-1Ra3, IL-1RAP, IL-17, or IRAK1. Moreover the compositions include morpholino antisense oligonucleotides, microRNAs (miRNAs), short hairpin RNA (shRNA), or short interfering RNA (siRNA) to silence gene expression, e.g., of IL-1α, IL-1β, IL-1R1, IL-1RAcP, or IL-17, or an IL-17 receptor. Exemplary compounds to be adapted for topical administration include, but are not limited to, anakinra/Kineret® (recombinant human IL-1Ra, rhIL-1Ra, and SEQ ID NO:16), IL-1R antisense oligomers (U.S. Patent No. 2005033694), IL-1Ra-like nucleic acid molecule (Amgen, U.S. Patent No. 2001041792), and polynucleotide encoding a soluble IL-1R accessory molecule (Human Genome Sciences, U.S. Issued Pat. No. 6,974,682).

Compositions of the methods of the invention include microRNA molecules adapted for topical administration to the cornea in order to silence gene expression. Exemplary miRNAs that bind to human IL-1α include, but are not limited to, miR-30c (SEQ ID NO: 34), miR-30b (SEQ ID NO: 35), miR-30a-5p (SEQ ID NO: 36), and miR-24 (SEQ ID NO: 37). Exemplary miRNAs (and corresponding sequences) that bind to human IL-1R1 include, but are not limited to, miR-135b (SEQ ID NO: 38), miR-326 (SEQ ID NO: 39), miR-184 (SEQ ID NO: 40), miR-214 (SEQ ID NO: 41), miR-203 (SEQ ID NO: 42), miR-331 (SEQ ID NO: 43), and miR-205 (SEQ ID NO: 44).

Exemplary polypeptides to be adapted for topical administration to the cornea include, but are not limited to, anakinra/Kineret® (recombinant human IL-1Ra, rhIL-1Ra, and SEQ ID NO:16), AF12198 (binds human IL-1R1, Ac-FEWTPGWYQJYALPL-NH2 where J represents the unnatural amino acid, 2-azetidine-1-carboxylic acid, SEQ ID NO: 45), IL-1R and IL-1RAP peptide antagonists (U.S. Patent No. 20060094663), IL-1R accessory molecule polypeptides (U.S. Patent No. 20050171337), IL-1Ra peptides (U.S. Patent No. 2005105830), and IL-1Ra-related peptides (Amgen, U.S. Patent No. 2001042304).

Exemplary antibodies to be adapted for topical administration to the cornea include, but are not limited to, IL-1 TRAP (inline fusion double chain protein of IL1R-gp130 with hIgGFc, Regeneron, U.S. Issued U.S. Pat. No. 6,927,044), anti-IL-1α (U.S. Patent No. 20030026806), anti-IL-1β (U.S. Patent No. 20030026806 and Yamasaki et al. Stroke. 1995; 26:676-681), and humanized monoclonal anti-IL-1R (Amgen, U.S. Patent No. 2004022718 and Roche, U.S. Patent No. 2005023872).

Small molecules are organic or inorganic. Exemplary organic small molecules include, but are not limited to, aliphatic hydrocarbons, alcohols, aldehydes, ketones, organic acids, esters, mono- and disaccharides, aromatic hydrocarbons, amino acids, and lipids. Exemplary inorganic small molecules comprise trace minerals, ions, free radicals, and metabolites. Alternatively, small molecule inhibitors can be synthetically engineered to consist of a fragment, or small portion, or a longer amino acid chain to fill a binding pocket of an enzyme. Typically small molecules are less than one kilodalton. An exemplary small molecule to be adapted for topical administration to the cornea is ZnPP (IL-1 blocker zinc protoporphyrin, naturally-occurring metabolite, Yamasaki et al. Stroke. 1995; 26:676-681).

Compositions of the methods of the invention include a polynucleotide, a polypeptide, an antibody, or a small molecule that binds or modifies the function of IL-1α, IL-1b, IL-1Ra, IL-1R1, IL-1R2, IL-1Ra3, IL-1RAP, IL-17, or IRAK1, administered topically with a pharmaceutically appropriate carrier. Delivery methods for polynucleotide compositions include, but are not limited to, liposomes, receptor-mediated delivery systems, naked DNA, and engineered viral vectors such as herpes viruses, retroviruses, adenoviruses and adeno-associated viruses, among others. Polynucleotide compositions are administered topically with a pharmaceutically acceptable liquid carrier, e.g., a liquid carrier, which is aqueous or partly aqueous. Alternatively, polynucleotide sequences within the composition are associated with a liposome (e.g., a cationic or anionic liposome).

A number of methods have been developed for delivering short DNA or RNA sequences into cells; e.g., polynucleotide molecules can be contacted directly onto the tissue site, or modified polynucleotide molecules, designed to specifically target desired cell types (e.g., sequences linked to peptides or antibodies that specifically bind receptors or antigens expressed on the target cell surface).

A preferred approach uses a recombinant DNA construct in which the short polynucleotide sequence is placed under the control of a strong polymerase III or polymerase II promoter. The use of such a construct will result in the transcription of sufficient amounts of polynucleotide that will form complementary base pairs with the endogenous transcripts of nucleic acids of the invention and thereby prevent translation of endogenous mRNA transcripts. The invention encompasses the construction of a short polynucleotide using the complementary strand as a template. For example, a vector can be introduced in vivo such that it is taken up by a cell and directs the transcription of an interfering RNA or precursor to a double stranded RNA molecule. Alternatively, the template for the short polynucleotide transcript is placed under the transcriptional control of a cell-type specific promoter or other regulatory element. Thus, diffusion or absorption of a topically administered composition beyond the cornea does not cause deleterious or systemic side effects. The vector remains episomal or becomes chromosomally integrated, as long as it can be transcribed to produce the desired polynucleotide.

Vectors are constructed by recombinant DNA technology methods standard in the art. Vectors can be plasmid, viral, or others known in the art, used for replication and expression in mammalian cells. Expression of the sequence encoding the short polynucleotide can be placed under the control of any promoter known in the art to act in mammalian, preferably human cells. Promoters are inducible or constitutive. Exemplary promoters include, but are not limited to: the SV40 early promoter region (Bernoist et al., Nature 290:304, 1981); the promoter contained in the 3′ long terminal repeat of Rous sarcoma virus (Yamamoto et al., Cell, 22:787-797, 1988); the herpes thymidine kinase promoter (Wagner et al., Proc. Natl. Acad. Sci. USA, 78:1441, 1981); or the regulatory sequences of the metallothionein gene (Brinster et al., Nature, 296:39, 1988).

Polypeptide compositions are associated with liposomes alone or in combination with receptor-mediated delivery systems, to enable transport across the plasma membrane. Polypeptide compositions are soluble or membrane-bound. An exemplary receptor-mediated delivery system involves fusion of a low-density or very-low-density lipoprotein containing particle or vesicle to the low-density lipoprotein (LDL) receptor (LDLR) as observed with Hepatitis C Virus (HCV) infection and HCV-mediated drug delivery methods.

Compositions of the methods of the invention include one or more extracellular or intracellular antibodies, also called intrabodies, raised against one or more of the following: IL-1α, IL-1b, IL-1Ra, IL-1R1, IL-1R2, IL-1Ra3, IL-1RAP, or IRAK1. Extracellular antibodies are topically administered with a pharmacologically appropriate aqueous or non-aqueous carrier. Sequences encoding intracellular antibodies are subcloned into a viral or mammalian expression vector, packed in a lipophilic device to facilitate transport across the plasma membrane, and topically administered to the cornea with a pharmacologically appropriate aqueous or non-aqueous carrier. Once inside the plasma membrane, host cell machinery transcribes, translates, and processes the intrabody code to generate an intracellular function-blocking antibody targeted against IL-1α, IL-1b, IL-1Ra, IL-1R1, IL-1R2, IL-1Ra3, IL-1RAP, or IRAK1. In the case of secreted molecules, intracellular antibodies prevent post-translational modification or secretion of the target protein. In the case of membrane-bound molecules, intracellular antibodies prevent intracellular signaling events upon receptor engagement by IL-1 cytokines.

In one preferred embodiment, methods of the invention includes a composition with means to inhibit the transcription, transcript stability, translation, modification, localization, secretion, or receptor binding of IL-1α, IL-1β, or a combination of both cytokines. In one embodiment, the composition comprises a polynucleotide capable of binding to a region of the IL-1α mRNA transcript, defined by SEQ ID NO: 1. In another embodiment, the composition comprises a polynucleotide capable of binding to a region of the IL-1β mRNA transcript, defined by SEQ ID NO: 3.

In another embodiment, the composition is capable of increasing the abundance of the naturally-occurring IL-1 Receptor antagonist (IL-1Ra). The composition comprises a polynucleotide, a polypeptide, an antibody, a compound, or a small molecule that binds to a region of the IL-1Ra gene, mRNA transcript defined by SEQ ID NO: 5, 7, 9, 11, or 13, a polypeptide isoform of IL-1Ra defined by SEQ ID NO: 6, 8, 10, 12, or 14, or a recombinant IL-1Ra protein defined by SEQ ID NO: 16. Alternatively, the composition contains mRNA transcripts or polypeptides encoding a region or the entirety of the IL-1Ra gene.

The composition includes an antagonist or inverse agonist of a receptor for IL-1α or IL-1β, specifically, IL-1R1. In this embodiment an antagonist is defined as a binding partner, or ligand, of an IL-1R that inhibits the function of an agonist, IL-1, or inverse agonist by blocking its binding to the receptor. An inverse agonist is defined as a molecule which binds to the same IL-1R binding-site as an agonist, for instance, IL-1, but exerts the opposite pharmacological effect. The composition contains a polynucleotide, a polypeptide, an antibody, a compound, or a small molecule that binds to a region of the IL-1R1 defined by the polynucleotide and polypeptide sequences SEQ ID NO: 17-21. In an alternative embodiment, the composition includes a molecule with means to inhibit IL-1R transcription, transcript stability, translation, modification, localization, secretion, ligand binding, or association with an accessory protein of an IL-1R (IL-1RAP). IL-1RAP is defined by the polynucleotide sequence of SEQ ID NO: 24 or 26 and the amino acid sequence of SEQ ID NO: 25 or 27.

In another preferred embodiment, the composition includes a human recombinant IL-1R antagonist either in pure form, or as a component of a mixture. The human recombinant IL-1R antagonist is combined with balanced saline, carboxymethylcellulose (CMC), or hyaluronic acid (HA), or other vehicles prior to the composition contacting the cornea. Within these mixtures, the human recombinant IL-1R antagonist comprises at least 0.1%, 2.0%, 2.5%, 5%, or at most 10% of the total volume administered. Preferred aqueous formulations contain 2-2.5% of the purified antagonist. Purified is defined as the antagonist in the absence of unrelated polynucleotides, polypeptides, cellular organelles, or lipids. Purified is defines a degree of sterility that is safe for administration to a human subject, e.g., lacking infectious or toxic agents.

All polynucleotides and polypeptides of the invention are purified and/or isolated. As used herein, an “isolated” or “purified” nucleic acid molecule, polynucleotide, polypeptide, or protein, is substantially free of other cellular material, or culture medium when produced by recombinant techniques, or chemical precursors or other chemicals when chemically synthesized. Purified compounds are at least 60% by weight (dry weight) the compound of interest. Preferably, the preparation is at least 75%, more preferably at least 90%, and most preferably at least 99%, by weight the compound of interest. Purity is measured by any appropriate standard method, for example, by column chromatography, polyacrylamide gel electrophoresis, or HPLC analysis.

Signs or symptoms of corneal damage or abnormal nerve morphology are detected, analyzed, examined, and evaluated using in vivo confocal microscopy (IVCM) of the central cornea or other imaging or diagnostic devices that allow for detection of corneal nerve damage. Exemplary devices for IVCM include, but are not limited to the Heidelberg Retina Tomograph 3 with the Rostock Cornea Module (HRT3/RCM)(Heidelberg Engineering GMBH) and the Confoscan 4 Confocal Microscope (Nidek, Inc.). In certain embodiments of the above methods, IVCM is used to detect, analyze, examine, and evaluate the form and number of nerve fibers in the various corneal layers, as well as to discriminate between parallel running, bifurcating, branching, and interconnecting nerve fiber bundles. Alternatively or in addition, IVCM is used to detect, analyze, examine, and evaluate changes in the total number of nerves, changes in the length of nerves, nerve density, the presence or absence of abnormal nerve sprouts, the presence or absence of abnormal nerve fiber tortuosity, changes in number or morphology of bead-like nerve formations, and thinning versus thickening of nerve fiber bundles. In one aspect of the methods of the invention, IVCM is used to detect, analyze, examine, and evaluate nerve regeneration. Alternatively, or in addition, IVCM is used to detect, analyze, examine, and evaluate nerve degeneration. For instance, IVCM has been used to show an average of 6-8 corneal nerve bundles per image within the subbasal area of healthy individuals and nerve regeneration in patients who experienced nerve damage as a result of photoreceptive keratectomy.

The invention also provides a method for reducing corneal nerve damage and/or enhancing corneal nerve regeneration in a subject in need thereof, including the steps of: (a) identifying a subject with corneal nerve damage; and (b) locally administering to the cornea of the subject a composition that inhibits an activity of an inflammatory interleukin-17 cytokine, thereby enhancing corneal nerve regeneration, reducing the development of abnormalities in nerve morphology, and reducing corneal nerve damage.

The invention also provides a method for protecting or regenerating corneal nerves in a subject in need thereof, comprising the steps of: (a) identifying a subject with corneal nerve damage or loss; and (b) locally administering to the cornea of the subject a composition that inhibits an activity of an inflammatory interleukin-1 cytokine and a composition that inhibits an activity of an inflammatory interleukin-17 cytokine, thereby enhancing corneal nerve regeneration and reducing the development of abnormalities in nerve morphology or density. This combination therapy leads to a synergistic effect in regenerating corneal nerve tissue.

Publications, U.S. patents and applications, Genbank/NCBI accession numbers, and all other references cited herein, are hereby incorporated by reference.

### BRIEF DESCRIPTION OF THE DRAWINGS

FIG. 1A is a microphotograph that shows the extent of terminal nerve branching system at the level of basal epithelial cells in a normal cornea of a Balb/c mouse.

FIG. 1B is a microphotograph of the terminal nerve processes following 7 days treatment with vehicle after epithelial debridement in a normal cornea of a Balb/c mouse, showing very minimal nerve regeneration activity at the level of the basal epithelial cells.

FIG. 1C is a microphotograph of the regenerated terminal nerve processes following 7 days treatment with 2.5% topical IL-1Ra after epithelial debridement in a normal cornea of a Balb/c mouse, showing a significant number of regenerate nerves at the level of basal epithelial cells, bringing the density of these nerves close to that seen in normal corneas (FIG. 1A).

FIG. 2 is a series of in vivo confocal images (Confoscan 4; Nidek Technologies) of subbasal corneal nerve before (left) and after (right) a one-month treatment with IL-1Ra 2.5% in a dry eye patient, showing an increase of 25% in nerve density after the treatment compared to the baseline.

FIG. 3 is a graph of the percent difference of corneal fluorescein staining observed in mouse models of dry eye treated with varying concentrations of IL-1Ra compared to untreated animals. As the graph shows, all concentrations of IL-1Ra (1%, 2.5%, and 5%) can decrease the corneal fluorescein staining score; however, the percent reduction of corneal fluorescein staining was modestly higher in the group that received topical IL-1Ra at a concentration of 5%.

FIG. 4 is a graph of the percent difference of corneal fluorescein staining observed in mouse models of dry eye treated with varying formulations of IL-1Ra compared to untreated animals. As the graph shows, the percent reduction of corneal fluorescein staining was highest in the groups that received topical IL-1Ra 5% mixed with N-acetyl cysteine 10% and the group that received topical IL-1Ra 5% mixed with carboxymethyl cellulose 1%.

FIG. 5 is a schematic representation of signaling pathways that are transduced from the IL-1R1 and the downstream effectors involved in carrying these intracellular signals (drawing reproduced from BioCarta website).

FIG. 6 is an in vivo confocal microscopic image (Confoscan 4; Nidek, Inc.) of subbasal nerve fibers in a healthy cornea of a 42-year-old male subject. Nerve bundles show a preferred orientation in the superior-inferior direction. Note the nerve fibers appear almost straight or slightly tortuous.

FIG. 7 is an in vivo confocal microscopic image (Confoscan 4; Nidek, Inc.) of subbasal nerve fibers in the cornea of a 56-year-old female subject with herpes zoster ophthalmicus. Note the significant decrease in the number of nerve bundles compared to the normal cornea. This microphotograph also shows signs of other nerve abnormalities such as high tortuosity, increased bead-like nerve formations, and an abnormal branching pattern.

FIG. 8A is a series of representative micrographs showing nerve fiber distribution in the central cornea of normal and dry eye disease (DED) mice. The white arrows show nerve fiber loss (reduced nerve length) in the cornea of dry eye mice. FIG. 8B is a bar diagram showing fold change (from normal cornea shown as horizontal solid arrow) in corneal nerve fiber length in DED mice treated topically with vehicle, IL-1Ra, anti-IL17-antibody, and cyclosporine-1 (CsA). FIG. 8C is a bar diagram showing fold change (from normal cornea shown as horizontal solid arrow) in corneal nerve fiber tortuosity in DED mice treated with vehicle, IL-1Ra, anti-IL17-antibody, and cyclosporine-1 (CsA).

FIG. 9A is a line graph demonstrating mean corneal fluorescein staining in 70 human patients with ocular surface inflammatory disorder and dry eyes treated with topical IL-1 receptor antagonist. FIG. 9B is a bar graph demonstrating the percent change from baseline of corneal staining in the same patients treated with topical IL-1 receptor antagonist.

FIG. 10A is a line graph demonstrating mean interpalpebral staining in 70 human patients with ocular surface inflammatory disorder and dry eyes treated with topical IL-1 receptor antagonist. FIG. 10B is a bar graph demonstrating the percent change from baseline of interpalpebral staining in the same patients treated with topical IL-1 receptor antagonist.

FIG. 11A is a line graph showing mean ocular surface disease index in 70 human patients with ocular surface inflammatory disorder and dry eyes treated with topical IL-1 receptor antagonist. FIG. 11B is a bar graph showing the percent change from baseline of ocular surface disease index in the same patients treated with topical IL-1 receptor antagonist.

FIG. 12 is a line graph demonstrating mean tear volume in 70 human patients with an ocular surface inflammatory disorder and dry eyes treated with topical IL-1 receptor antagonist.

FIG. 13 is a series of photomicrographs depicting a corneal nerve regeneration in the human patients with ocular surface inflammatory disorder and dry eyes treated with topical IL-1 receptor antagonist or vehicle (lubricating eye drops).

### DETAILED DESCRIPTION

IL-1, particularly IL-1β, has been reported to promote nerve regeneration. Earlier studies reported that IL-1β was upregulated or stimulated production of the neurotrophin, nerve growth factor (NGF) (Pons et al., 2002, Eur. Respir. J. 20:458-463; Akeda et al., 2007, Spine 32:635-642). For example, IL-1β inhibition using IL-1Ra was found to suppress a neurotrophin response in injured brain tissue. An increase in nerve growth factor (NGF) was found to be directly mediated through IL-1β, and blocking IL-1β with IL-1Ra led to suppression of the NGF-mediated reparative response (DeKosky et al., 1996, Ann. Neurol. 39:123-127). The data reported herein indicate that IL-1 blockade stimulates corneal nerve regeneration, an unexpected and surprising finding that contradicts these earlier reports.

IL-1 blockade was used to treat patients characterized by complaints of chronic ocular irritation and discomfort. Using an animal model and clinical studies, compositions and methods of the invention demonstrate that corneal nerves are protected and indeed regenerated by inhibiting the action of IL-1. Specifically, IL-1 blockade through topical administration of IL-1 Receptor antagonist (IL-1Ra), which acts as an antagonist to IL-1, protects corneal nerves, enhances corneal nerve regeneration, and reduces the abnormalities in subbasal nerve morphology.

Exemplary abnormalities in subbasal nerve morphology include, but are not limited to the presence of abnormal nerve sprouts, abnormal tortuosity, increased bead-like formation, and thinning or thickening of nerve fiber bundles. Thus, IL-1 inhibitors are protective in neuropathic conditions such as herpes simplex or zoster keratitis, diabetes mellitus, dry eye, exposure keratopathy, trigeminal nerve damage associated with orbital or head surgery, head trauma, aneurysms, or intracranial neurologic disease, and corneal nerve damage associated with keratorefractive procedures such as PRK and LASIK.

Corneal nerves are characterized by unique anatomical location, structural features, and functions, compared to other nerves, e.g., the cornea is an avascular location and has unmyelinated nerve endings sensitive to touch, temperature and chemicals. A touch of the cornea or other stimulus causes an involuntary reflex to close the eyelid.

### Corneal Structure

The cornea is the transparent front part of the eye that covers the iris, pupil, and anterior chamber. Together with the lens, the cornea refracts light, and as a result helps the eye to focus, accounting for approximately two-thirds of the eye's total optical power. The cornea has unmyelinated nerve endings sensitive to touch, temperature and chemicals; a touch of the cornea causes an involuntary reflex to close the eyelid.

Because transparency is of prime importance the cornea does not have blood vessels; it receives nutrients via diffusion from the tear fluid at the outside and the aqueous humor at the inside and also from neurotrophins supplied by nerve fibers that innervate it. In humans, the cornea has a diameter of about 11.5 mm and a thickness of 0.5-0.6 mm in the center and 0.6-0.8 mm at the periphery. Transparency, avascularity, the presence of highly immature resident immune cells, and immunologic privilege makes the cornea a unique tissue. Immune privilege is meant to describe certain sites in the body that are able to tolerate the introduction of an antigen without eliciting an inflammatory immune response. The cornea has no blood supply, but rather, the cornea receives oxygen directly through the air and the tears that bathe it.

The human cornea, like that of other primates, has five layers. From the anterior to posterior they are the corneal epithelium, Bowman's layer, the corneal stroma, Descemet's membrane, and the corneal endothelium. The corneal epithelium is a thin epithelial multicellular tissue layer, stratified squamous epithelium, of continuously regenerating cells, kept moist with tears. Irregularity or edema of the corneal epithelium disrupts the smoothness of the air-tear film interface, the most significant component of the total refractive power of the eye, thereby reducing visual acuity. Bowman's layer, also known as the anterior limiting membrane, is a condensed layer of irregularly-arranged collagen, about 8-14 microns thick, that protects the corneal stroma. The corneal stroma, also known as the substantia propria, is a thick and transparent middle layer, consisting of regularly-arranged collagen fibers along with sparsely populated keratocytes. The corneal stroma consists of approximately 200 layers of type I collagen fibrils. Ninety percent of the corneal thickness is composed of the stroma. Descemet's membrane, also known as the posterior limiting membrane, is a thin and acellular layer that serves as the modified basement membrane of the corneal endothelium. The corneal endothelium is a simple squamous or low cuboidal monolayer of mitochondria-rich cells responsible for regulating fluid and solute transport between the aqueous and corneal stromal compartments. The corneal endothelium is bathed by aqueous humour, not by blood or lymph, and has a very different origin, function, and appearance from vascular endothelia. Unlike the corneal epithelium, the cells of the endothelium do not regenerate. Instead, corneal endothelial cells expand or spread to compensate for dead cells which reduces the overall cell density of the endothelium and impacts fluid regulation.

The cornea is one of the most sensitive tissues of the body, it is densely innervated with sensory nerve fibers via the ophthalmic division of the trigeminal nerve by way of 70-80 long and short ciliary nerves. Nerves enter the cornea via three levels, scleral, episcleral and conjunctival. Most of the bundles subdivide and form a network in the stroma, from which fibers supply different regions of the cornea. Three exemplary networks are midstromal, subepithelial/Bowman's layer, and epithelium. Corneal nerves of the subepithelial layer converge and terminate near the apex of the cornea.

### Corneal Innervation

The cornea is one of the most densely innervated tissues in the body and is abundantly supplied by different types of nerve fibers. Rabbit studies have revealed that the nerve density of the corneal epithelium is about 300-600 times as much as that of skin and 20-40 times that of the dental pulp. It is estimated that there are approximately 7000 sensory receptors per mm² in the human corneal epithelium, implying that injuries to individual epithelial cells may be adequate to give a pain perception (Müller et al., Exp Eye Res 2003; 76:521-42).

Most corneal nerve fibers are sensory in origin and are derived from the ophthalmic branch of the trigeminal nerve. Nerve bundles enter the peripheral mid-stromal cornea in a radial fashion parallel to the corneal surface. Soon after entering the cornea, the main stromal bundles branch repeatedly and dichotomously into smaller fascicles that ascended into progressively more superficial layers of the stroma. Eventually, the stromal nerve fibers turn abruptly 90°, penetrate Bowman's layer and proceed towards the corneal surface. After penetrating Bowman's layer, bundles divide and run parallel to the corneal surface between Bowman's layer and the basal epithelium, forming the subbasal nerve plexus. The density and number of nerves in the subbasal epithelial nerve plexus are significantly greater than the density and number of nerves in the remaining corneal layers. Subbasal fibers subsequently form branches that turn upward and enter the corneal epithelium between the basal cells to reach the wing cells, where they terminate (Müller et al., Invest Ophthalmol Vis Sci 1996; 37:476-88).

Corneal nerve fibers mediate not only sensation but also exert critical trophic influences on the corneal epithelium and play a vital role to the preservation of a healthy ocular surface. Corneal sensation is a key mechanism in preventing injury through the blink reflex and reflex tearing. Neuropathy, e.g., degeneration of corneal nerves, leads to changes in sensation. Patients diagnosed with neuropathy of the corneal nerve experience diminished sensation and/or increased pain (hyperalgesia), characterized by chronic discomfort and irritation. Since lacrimation is regulated by the corneal nerves, corneal neuropathy (loss or damaged corneal nerve tissue or decreased length of corneal nerve fibers) leads to tear deficiency. Thus, the methods are useful to reduce the symptoms of tear-deficient dry eye.

Dysfunction of corneal innervation and related neuropathic pathology produces a degenerative condition known clinically as “neurotrophic keratitis”, which therefore renders the corneal surface vulnerable to occult injury and delayed healing of established corneal epithelial injuries. Most clinical cases of neurotrophic keratitis are caused by herpes simplex or zoster keratitis, diabetes mellitus, or by trigeminal nerve damage associated with orbital or head surgery, head trauma, aneurysms, or intracranial neurologic disease. Absent or reduced corneal sensation may be congenital in origin. Keratorefractive procedures such as photorefractive keratectomy (PRK) and laser in situ keratomileusis (LASIK) can sever stromal and subbasal corneal nerves plexus and produce a transient mild to severe neuropathologic condition, or neurotrophic dry eye. This form of “neurotrophic dry eye”, characterized by nerve loss and associated dryness, is also seen in non-surgical conditions such as severe forms of dry eye that develop in subjects.

Intact corneal innervation is also mandatory for tearing reflexes. Under normal physiological conditions, sensory nerves in the cornea transmit an afferent stimulation signal to the brain stem and then, after a series of interneurons, the efferent signal is transmitted to the lacrimal gland through the parasympathetic and sympathetic nerves that innervate the gland and drive tear production and secretion (Dartt, D A Ocul Surf 2004; 2:76-91). Damage to this neural circuit interrupts the normal regulation of lacrimal gland secretion and causes dry eye disease. A reduction in neural drive from the cornea favors the occurrence of dry eye-associated ocular surface disease in two ways; first, by decreasing reflex-induced lacrimal secretion and by reducing the blink rate and, consequently, increasing evaporative loss; second, by decreasing the trophic factors to the epithelial layer. Damage to the sensory nerves in the ocular surface, particularly the cornea, as a consequence of refractive surgery and normal aging, prevents the normal reflex arc to the lacrimal gland and can result in decreased tear secretion and dry eye syndromes. Evidence for this mechanism comes from the clinical observation that dry eye syndrome frequently occurs after corneal refractive surgery (e.g., surgery in which the nerve is transected). Clinical studies confirmed that tear production and secretion are reduced after LASIK surgery (Battat et al., Ophthalmology 2001; 108:1230-5). Interestingly, hyposecretion of tears in dry eye may lead to pathologic alterations in corneal nerves and a decline in corneal sensitivity which subsequently perpetuate the dry eye state (Xu et al., Cornea 1996; 15:235-9). Dry eye is further described in PCT/US2008/009776, which is incorporated herein by reference. In addition, inflammation of the corneal surface can lead to increases in inflammatory IL-1 cytokines that alter nociception on IL-1 receptor expressing corneal nerves.

Patients with only meibomian gland disease (MGD) or posterior blepharitis are generally not characterized as having clinical neuropathy (clinically significant corneal nerve damage), and hence do not have “neurotrophic” dry eye.

### Corneal Pathology

Ocular diseases that affect the corneal epithelium such as dry eye, exposure keratopathy, and other ocular surface diseases cause corneal nerve degeneration. On the other hand, normal neural drive is an essential requirement for corneal epithelium to heal and maintain its homeostasis. Therefore, corneal nerve alterations, either as a primary reason (refractive surgery) or just as the outcome of dryness and other corneal epithelial or ocular surface diseases, have crucial effects on the homeostasis of corneal epithelium, thus neatly contributing to the increase of the vicious cycle of epithelial disease and nerve damage.

### Interleukin-1 (IL-1)

The IL-1 family is a group of cytokines that function as major mediators of inflammation and immune response (Dinarello, C. A. 1996. Blood. 15:2095-2147). This family is composed of three forms: two proinflammatory forms, IL-1α and IL-1β, each having a precursor form, and an anti-inflammatory form, IL-1 receptor antagonist (IL-1Ra). The proinflammatory cytokine IL-1 plays an important role in inflammation and immunity by increasing chemokine production, adhesion factors, macrophage infiltration and activity, and lymphocyte proliferation. IL-1 has been implicated in the pathogenesis of human inflammatory diseases, such as rheumatoid arthritis, septic shock, and periodontitis (Jiang, Y. et al. 2000. Arthritis Rheum. 43:1001-1009; Okusawa, S. et al. 1988. J Clin Invest. 81: 1162-1172; McDevitt, M. J. et al. 2000. J. Periodontol. 71:156-163).

The compositions and methods described herein inhibit the activity of human IL-1α and/or IL-1β, as defined by the ability to induce signal transduction or initiate/activate a downstream signaling cascade from an IL-1 receptor. Compositions that contain an inhibitor of human IL-1α or IL-1β function antagonize the activity of an IL-1 receptor. The composition comprises a polynucleotide, a polypeptide, an antibody, a compound, or a small molecule with means to inhibit the transcription, transcript stability, translation, modification, localization, secretion, or function of a polynucleotide or polypeptide encoding human IL-1α or IL-1β. Moreover, the inhibitory polynucleotide or polypeptide composition binds to one or more region(s) of IL-1α or IL-1β comprised by SEQ ID NO: 1 and SEQ ID NO: 2 (IL-1α) or SEQ ID NO: 3 and SEQ ID NO: 4 (IL-1β). The inhibitory polynucleotide or polypeptide composition binds to one or more fragments of IL-1α or IL-1β comprised by SEQ ID NO: 1 and SEQ ID NO: 2 (IL-1α) or SEQ ID NO: 3 and SEQ ID NO: 4 (IL-1β).

Exemplary IL-1 inhibitors include peptides that bind to and inhibit IL-1R1, such as those described in U.S. Pat. No. 5,861,476. Peptides can bind, e.g., with an affinity of less than 500 nM, 50 nM, or 1 nM. Peptides can include between 8 and 40 amino acids, e.g., between 10-30 amino acids. Methods for synthesizing peptides are described, e.g., in Peptide Synthesis and Applications (Howl, ed.), Humana Press (2010) (ISBN: 1617374903).

Other exemplary IL-1 inhibitors that bind to IL-1β and, e.g., antagonize IL-1β activity. For example such proteins can prevent interaction between IL-1β and its receptors, e.g., IL-1R1. Proteins in this category include soluble forms of IL-1R1 receptors and antibodies that bind IL-1β. Exemplary antibodies that bind to IL-1β include XOMA-052 and canakinumab, as well as Fv, Fab and scFv fragments and other variants thereof.

Additional IL-1 inhibitors include proteins that bind to IL-1α and, e.g., antagonize IL-1α activity. Such proteins can prevent interaction between IL-1α and its receptors, e.g., IL-1RI. Proteins in this category include soluble forms of IL-1RI receptors and antibodies that bind IL-1α. Still other antagonists are bispecific antibodies that recognize IL-1α and IL-1β. See, e.g., U.S. Pat. No. 7,612,181.

Further exemplary IL-1 inhibitors include proteins that bind to one or both of IL-1β and IL-1α, including antibodies having specificity for both these cytokines and soluble forms of receptors for IL-1 cytokines, e.g., soluble forms of IL-1R1 and IL-1R2, and IL-1RAcP, such as rilonacept.

A fragment, in the case of these sequences and all others provided herein, is defined as a part of the whole that is less than the whole. Moreover, a fragment ranges in size from a single nucleotide or amino acid within a polynucleotide or polypeptide sequence to one fewer nucleotide or amino acid than the entire polynucleotide or polypeptide sequence. Finally, a fragment is defined as any portion of a complete polynucleotide or polypeptide sequence that is intermediate between the extremes defined above.

Human IL-1α has the following mRNA sequence (NCBI Accession No. NM_{—}000575 and SEQ ID NO: 1): (For all mRNA transcripts incorporated into the present application, the initiator methionine, encoded by the codon “atg,” is bolded and capitalized to delineate the start of the coding region.)

accaggcaacaccattgaaggctcatatgtaaaaatccatgccttcctttctcccaatctccattcccaa
acttagccactggcttctggctgaggccttacgcatacctcccggggcttgcacacaccttcttctacag
aagacacaccttgggcatatcctacagaagaccaggcttctctctggtccttggtagagggctactttac
tgtaacagggccagggtggagagttctctcctgaagctccatcccctctataggaaatgtgttgacaata
ttcagaagagtaagaggatcaagacttctttgtgctcaaataccactgttctcttctctaccctgcccta
accaggagcttgtcaccccaaactctgaggtgatttatgccttaatcaagcaaacttccctcttcagaaa
agatggctcattttccctcaaaagttgccaggagctgccaagtattctgccaattcaccctggagcacaa
tcaacaaattcagccagaacacaactacagctactattagaactattattattaataaattcctctccaa
atctagccccttgacttcggatttcacgatttctcccttcctcctagaaacttgataagtttcccgcgct
tccctttttctaagactacatgtttgtcatcttataaagcaaaggggtgaataaatgaaccaaatcaata
acttctggaatatctgcaaacaacaataatatcagctatgccatctttcactattttagccagtatcgag
ttgaatgaacatagaaaaatacaaaactgaattcttccctgtaaattccccgttttgacgacgcacttgt
agccacgtagccacgcctacttaagacaattacaaaaggcgaagaagactgactcaggcttaagctgcca
gccagagagggagtcatttcattggcgtttgagtcagcaaagaagtcaagATGgccaaagttccagacat
gtttgaagacctgaagaactgttacagtgaaaatgaagaagacagttcctccattgatcatctgtctctg
aatcagaaatccttctatcatgtaagctatggcccactccatgaaggctgcatggatcaatctgtgtctc
tgagtatctctgaaacctctaaaacatccaagcttaccttcaaggagagcatggtggtagtagcaaccaa
cgggaaggttctgaagaagagacggttgagtttaagccaatccatcactgatgatgacctggaggccatc
gccaatgactcagaggaagaaatcatcaagcctaggtcagcaccttttagcttcctgagcaatgtgaaat
acaactttatgaggatcatcaaatacgaattcatcctgaatgacgccctcaatcaaagtataattcgagc
caatgatcagtacctcacggctgctgcattacataatctggatgaagcagtgaaatttgacatgggtgct
tataagtcatcaaaggatgatgctaaaattaccgtgattctaagaatctcaaaaactcaattgtatgtga
ctgcccaagatgaagaccaaccagtgctgctgaaggagatgcctgagatacccaaaaccatcacaggtag
tgagaccaacctcctcttcttctgggaaactcacggcactaagaactatttcacatcagttgcccatcca
aacttgtttattgccacaaagcaagactactgggtgtgcttggcaggggggccaccctctatcactgact
ttcagatactggaaaaccaggcgtaggtctggagtctcacttgtctcacttgtgcagtgttgacagttca
tatgtaccatgtacatgaagaagctaaatcctttactgttagtcatttgctgagcatgtactgagccttg
taattctaaatgaatgtttacactctttgtaagagtggaaccaacactaacatataatgttgttatttaa
agaacaccctatattttgcatagtaccaatcattttaattattattcttcataacaattttaggaggacc
agagctactgactatggctaccaaaaagactctacccatattacagatgggcaaattaaggcataagaaa
actaagaaatatgcacaatagcagttgaaacaagaagccacagacctaggatttcatgatttcatttcaa
ctgtttgccttctacttttaagttgctgatgaactcttaatcaaatagcataagtttctgggacctcagt
tttatcattttcaaaatggagggaataatacctaagccttcctgccgcaacagttttttatgctaatcag
ggaggtcattttggtaaaatacttcttgaagccgagcctcaagatgaaggcaaagcacgaaatgttattt
tttaattattatttatatatgtatttataaatatatttaagataattataatatactatatttatgggaa
ccccttcatcctctgagtgtgaccaggcatcctccacaatagcagacagtgttttctgggataagtaagt
ttgatttcattaatacagggcattttggtccaagttgtgcttatcccatagccaggaaactctgcattct
agtacttgggagacctgtaatcatataataaatgtacattaattaccttgagccagtaattggtccgatc
tttgactcttttgccattaaacttacctgggcattcttgtttcaattccacctgcaatcaagtcctacaa
gctaaaattagatgaactcaactttgacaaccatgagaccactgttatcaaaactttcttttctggaatg
taatcaatgtttcttctaggttctaaaaattgtgatcagaccataatgttacattattatcaacaatagt
gattgatagagtgttatcagtcataactaaataaagcttgcaacaaaattctctgacaaaaaaaaaaaaa
aaa.

Human IL-1α encodes the following amino acid sequence (NCBI Accession No. NM_{—}000575 and SEQ ID NO: 2):

MAKVPDMFEDLKNCYSENEEDSSSIDHLSLNQKSFYHVSYGPLHDSEEEI
IKPRSAPFSFLSNVKYNFMRIIKYEFILNDALNQSIIRANDQYLTAAALH
NLDEAVKFDMGAYKSSKDDAKITVILRISKTQLYVTAQDEDQPVLLKEMP
EIPKTITGSETNLLFFWETHGTKNYFTSVAHPNLFIATKQDYWVCLAGGP
PSITDFQILENQA.

Human IL-1β has the following mRNA sequence (NCBI Accession No. NM_{—}000576 and SEQ ID NO: 3):

accaaacctcttcgaggcacaaggcacaacaggctgctctgggattctcttcagccaatcttcattgctc
aagtgtctgaagcagccATGgcagaagtacctgagctcgccagtgaaatgatggcttattacagtggcaa
tgaggatgacttgttctttgaagctgatggccctaaacagatgaagtgctccttccaggacctggacctc
tgccctctggatggcggcatccagctacgaatctccgaccaccactacagcaagggcttcaggcaggccg
cgtcagttgttgtggccatggacaagctgaggaagatgctggttccctgcccacagaccttccaggagaa
tgacctgagcaccttctttcccttcatctttgaagaagaacctatcttcttcgacacatgggataacgag
gcttatgtgcacgatgcacctgtacgatcactgaactgcacgctccgggactcacagcaaaaaagcttgg
tgatgtctggtccatatgaactgaaagctctccacctccagggacaggatatggagcaacaagtggtgtt
ctccatgtcctttgtacaaggagaagaaagtaatgacaaaatacctgtggccttgggcctcaaggaaaag
aatctgtacctgtcctgcgtgttgaaagatgataagcccactctacagctggagagtgtagatcccaaaa
attacccaaagaagaagatggaaaagcgatttgtcttcaacaagatagaaatcaataacaagctggaatt
tgagtctgcccagttccccaactggtacatcagcacctctcaagcagaaaacatgcccgtcttcctggga
gggaccaaaggcggccaggatataactgacttcaccatgcaatttgtgtcttcctaaagagagctgtacc
cagagagtcctgtgctgaatgtggactcaatccctagggctggcagaaagggaacagaaaggtttttgag
tacggctatagcctggactttcctgttgtctacaccaatgcccaactgcctgccttagggtagtgctaag
aggatctcctgtccatcagccaggacagtcagctctctcctttcagggccaatccccagcccttttgttg
agccaggcctctctcacctctcctactcacttaaagcccgcctgacagaaaccacggccacatttggttc
taagaaaccctctgtcattcgctcccacattctgatgagcaaccgcttccctatttatttatttatttgt
ttgtttgttttattcattggtctaatttattcaaagggggcaagaagtagcagtgtctgtaaaagagcct
agtttttaatagctatggaatcaattcaatttggactggtgtgctctctttaaatcaagtcctttaatta
agactgaaaatatataagctcagattatttaaatgggaatatttataaatgagcaaatatcatactgttc
aatggttctgaaataaacttcactgaag.

Human IL-1β encodes the following amino acid sequence (NCBI Accession No. NM_{—}000576 and SEQ ID NO: 4):

MAEVPELASEMMAYYSGNEDDLFFEADGPKQMKCSFQDLDLCPLDGGI
QLRISDHHYSKGFRQAASVVVAMDKLRKMLVPCPQTFQENDLSTFFPFIF
EEEPIFFDTWDNEAYVHDAPVRSLNCTLRDSQQKSLVMSGPYELKALHL
QGQDMEQQVVFSMSFVQGEESNDKIPVALGLKEKNLYLSCVLKDDKPTL
QLESVDPKNYPKKKMEKRFVFNKIEINNKLEFESAQFPNWYISTSQAEN
MPVFLGGTKGGQDITDFTMQFVSS.

### Interleukin-1 Receptor (Type 1) Antagonist (IL-1Ra):

IL-1Ra is an endogenous receptor antagonist which is primarily produced by activated monocytes and tissue macrophages, inhibits the activities of the proinflammatory forms of IL-1 by competitively binding to IL-1 receptor. (Gabay, C. et al. 1997. 159: 5905-5913). IL-1Ra is an inducible gene that is typically upregulated in inflammatory conditions (Arend, W. P. 1993. Adv Immunol. 54: 167-223).

In the present invention, compositions comprise one or more regions of IL-1Ra transcripts 1, 2, 3, or 4, intacellular IL-1Ra (icIL-1Ra), or their corresponding polypeptide isoforms. Alternatively, compositions comprise the entirety of IL-1Ra transcripts 1, 2, 3, or 4, intacellular IL-1Ra (icIL-1Ra), or their corresponding polypeptide isoforms. Compositions comprising any form of human IL-1Ra, or fragments thereof, inhibit the function of IL-1R1. These polynucleotides and polypeptides are defined by the following sequences. Human IL-1Ra, transcript 1, has the following mRNA sequence (NCBI Accession No. NM_{—}173842 and SEQ ID NO: 5):

atttctttataaaccacaactctgggcccgcaatggcagtccactgccttgctgcagtcacagaATGgaa
atctgcagaggcctccgcagtcacctaatcactctcctcctcttcctgttccattcagagacgatctgcc
gaccctctgggagaaaatccagcaagatgcaagccttcagaatctgggatgttaaccagaagaccttcta
tctgaggaacaaccaactagttgctggatacttgcaaggaccaaatgtcaatttagaagaaaagatagat
gtggtacccattgagcctcatgctctgttcttgggaatccatggagggaagatgtgcctgtcctgtgtca
agtctggtgatgagaccagactccagctggaggcagttaacatcactgacctgagcgagaacagaaagca
ggacaagcgcttcgccttcatccgctcagacagcggccccaccaccagttttgagtctgccgcctgcccc
ggttggttcctctgcacagcgatggaagctgaccagcccgtcagcctcaccaatatgcctgacgaaggcg
tcatggtcaccaaattctacttccaggaggacgagtagtactgcccaggcctgcctgttcccattcttgc
atggcaaggactgcagggactgccagtccccctgccccagggctcccggctatgggggcactgaggacca
gccattgaggggtggaccctcagaaggcgtcacaagaacctggtcacaggactctgcctcctcttcaact
gaccagcctccatgctgcctccagaatggtctttctaatgtgtgaatcagagcacagcagcccctgcaca
aagcccttccatgtcgcctctgcattcaggatcaaaccccgaccacctgcccaacctgctctcctcttgc
cactgcctcttcctccctcattccaccttcccatgccctggatccatcaggccacttgatgacccccaac
caagtggctcccacaccctgttttacaaaaaagaaaagaccagtccatgagggaggtttttaagggtttg
tggaaaatgaaaattaggatttcatgatttttttttttcagtccccgtgaaggagagcccttcatttgga
gattatgttctttcggggagaggctgaggacttaaaatattcctgcatttgtgaaatgatggtgaaagta
agtggtagcttttcccttctttttcttctttttttgtgatgtcccaacttgtaaaaattaaaagttatgg
tactatgttagccccataattttttttttccttttaaaacacttccataatctggactcctctgtccagg
cactgctgcccagcctccaagctccatctccactccagattttttacagctgcctgcagtactttacctc
ctatcagaagtttctcagctcccaaggctctgagcaaatgtggctcctgggggttctttcttcctctgct
gaaggaataaattgctccttgacattgtagagcttctggcacttggagacttgtatgaaagatggctgtg
cctctgcctgtctcccccaccgggctgggagctctgcagagcaggaaacatgactcgtatatgtctcagg
tccctgcagggccaagcacctagcctcgctcttggcaggtactcagcgaatgaatgctgtatatgttggg
tgcaaagttccctacttcctgtgacttcagctctgttttacaataaaatcttgaaaatgcctaaaaaaaa
aaaaaaaaaa.

Human IL-1Ra, transcript 1, encodes the following amino acid sequence (NCBI Accession No. NM_{—}173842 and SEQ ID NO: 6):

MEICRGLRSHLITLLLFLFHSETICRPSGRKSSKMQAFRIWDVNQKTF
YLRNNQLVAGYLQGPNVNLEEKIDVVPIEPHALFLGIHGGKMCLSCVK
SGDETRLQLEAVNITDLSENRKQDKRFAFIRSDSGPTTSFESAACPG
WFLCTAMEADQPVSLTNMPDEGVMVTKFYFQEDE.

Human IL-1Ra, transcript 2, has the following mRNA sequence (NCBI Accession No. NM_{—}173841 and SEQ ID NO: 7):

gggcagctccaccctgggagggactgtggcccaggtactgcccgggtgctactttatgggcagcagctca
gttgagttagagtctggaagacctcagaagacctcctgtcctatgaggccctccccATGgctttagctga
cttgtatgaagaaggaggtggaggaggaggagaaggtgaagacaatgctgactcaaaggagacgatctgc
cgaccctctgggagaaaatccagcaagatgcaagccttcagaatctgggatgttaaccagaagaccttct
atctgaggaacaaccaactagttgctggatacttgcaaggaccaaatgtcaatttagaagaaaagataga
tgtggtacccattgagcctcatgctctgttcttgggaatccatggagggaagatgtgcctgtcctgtgtc
aagtctggtgatgagaccagactccagctggaggcagttaacatcactgacctgagcgagaacagaaagc
aggacaagcgcttcgccttcatccgctcagacagcggccccaccaccagttttgagtctgccgcctgccc
cggttggttcctctgcacagcgatggaagctgaccagcccgtcagcctcaccaatatgcctgacgaaggc
gtcatggtcaccaaattctacttccaggaggacgagtagtactgcccaggcctgcctgttcccattcttg
catggcaaggactgcagggactgccagtccccctgccccagggctcccggctatgggggcactgaggacc
agccattgaggggtggaccctcagaaggcgtcacaagaacctggtcacaggactctgcctcctcttcaac
tgaccagcctccatgctgcctccagaatggtctttctaatgtgtgaatcagagcacagcagcccctgcac
aaagcccttccatgtcgcctctgcattcaggatcaaaccccgaccacctgcccaacctgctctcctcttg
ccactgcctcttcctccctcattccaccttcccatgccctggatccatcaggccacttgatgacccccaa
ccaagtggctcccacaccctgttttacaaaaaagaaaagaccagtccatgagggaggtttttaagggttt
gtggaaaatgaaaattaggatttcatgatttttttttttcagtccccgtgaaggagagcccttcatttgg
agattatgttctttcggggagaggctgaggacttaaaatattcctgcatttgtgaaatgatggtgaaagt
aagtggtagcttttcccttctttttcttctttttttgtgatgtcccaacttgtaaaaattaaaagttatg
gtactatgttagccccataattttttttttccttttaaaacacttccataatctggactcctctgtccag
gcactgctgcccagcctccaagctccatctccactccagattttttacagctgcctgcagtactttacct
cctatcagaagtttctcagctcccaaggctctgagcaaatgtggctcctgggggttctttcttcctctgc
tgaaggaataaattgctccttgacattgtagagcttctggcacttggagacttgtatgaaagatggctgt
gcctctgcctgtctcccccaccgggctgggagctctgcagagcaggaaacatgactcgtatatgtctcag
gtccctgcagggccaagcacctagcctcgctcttggcaggtactcagcgaatgaatgctgtatatgttgg
gtgcaaagttccctacttcctgtgacttcagctctgttttacaataaaatcttgaaaatgcctaaaaaaa
aaaaaaaaaaaaaaaaaaaaaaaaaaaaaaaaaaaaaaaaaaaaa.

Human IL-1Ra, transcript 2, encodes the following amino acid sequence (NCBI Accession No. NM_{—}173841 and SEQ ID NO: 8):

MALADLYEEGGGGGGEGEDNADSKETICRPSGRKSSKMQAFRIWDVNQK
TFYLRNNQLVAGYLQGPNVNLEEKIDVVPIEPHALFLGIHGGKMCLSCVK
SGDETRLQLEAVNITDLSENRKQDKRFAFIRSDSGPTTSFESAACPGWFL
CTAMEADQPVSLTNMPDEGVMVTKFYFQEDE.

Human IL-1Ra, transcript 3, has the following mRNA sequence (NCBI Accession No. NM_{—}000577 and SEQ ID NO: 9):

gggcagctccaccctgggagggactgtggcccaggtactgcccgggtgctactttatgggcagcagctca
gttgagttagagtctggaagacctcagaagacctcctgtcctatgaggccctccccATGgctttagagac
gatctgccgaccctctgggagaaaatccagcaagatgcaagccttcagaatctgggatgttaaccagaag
accttctatctgaggaacaaccaactagttgctggatacttgcaaggaccaaatgtcaatttagaagaaa
agatagatgtggtacccattgagcctcatgctctgttcttgggaatccatggagggaagatgtgcctgtc
ctgtgtcaagtctggtgatgagaccagactccagctggaggcagttaacatcactgacctgagcgagaac
agaaagcaggacaagcgcttcgccttcatccgctcagacagcggccccaccaccagttttgagtctgccg
cctgccccggttggttcctctgcacagcgatggaagctgaccagcccgtcagcctcaccaatatgcctga
cgaaggcgtcatggtcaccaaattctacttccaggaggacgagtagtactgcccaggcctgcctgttccc
attcttgcatggcaaggactgcagggactgccagtccccctgccccagggctcccggctatgggggcact
gaggaccagccattgaggggtggaccctcagaaggcgtcacaagaacctggtcacaggactctgcctcct
cttcaactgaccagcctccatgctgcctccagaatggtctttctaatgtgtgaatcagagcacagcagcc
cctgcacaaagcccttccatgtcgcctctgcattcaggatcaaaccccgaccacctgcccaacctgctct
cctcttgccactgcctcttcctccctcattccaccttcccatgccctggatccatcaggccacttgatga
cccccaaccaagtggctcccacaccctgttttacaaaaaagaaaagaccagtccatgagggaggttttta
agggtttgtggaaaatgaaaattaggatttcatgatttttttttttcagtccccgtgaaggagagccctt
catttggagattatgttctttcggggagaggctgaggacttaaaatattcctgcatttgtgaaatgatgg
tgaaagtaagtggtagcttttcccttctttttcttctttttttgtgatgtcccaacttgtaaaaattaaa
agttatggtactatgttagccccataattttttttttccttttaaaacacttccataatctggactcctc
tgtccaggcactgctgcccagcctccaagctccatctccactccagattttttacagctgcctgcagtac
tttacctcctatcagaagtttctcagctcccaaggctctgagcaaatgtggctcctgggggttctttctt
cctctgctgaaggaataaattgctccttgacattgtagagcttctggcacttggagacttgtatgaaaga
tggctgtgcctctgcctgtctcccccaccgggctgggagctctgcagagcaggaaacatgactcgtatat
gtctcaggtccctgcagggccaagcacctagcctcgctcttggcaggtactcagcgaatgaatgctgtat
atgttgggtgcaaagttccctacttcctgtgacttcagctctgttttacaataaaatcttgaaaatgcct
aaaaaaaaaaaaaaaaaaaaaaaaaaaaaaaaaaaaaaaaaaaaaaaaaaaa.

Human IL-1Ra, transcript 3, encodes the following amino acid sequence (NCBI Accession No. NM_{—}000577 and SEQ ID NO: 10):

MALETICRPSGRKSSKMQAFRIWDVNQKTFYLRNNQLVAGYLQGPNVNL
EEKIDVVPIEPHALFLGIHGGKMCLSCVKSGDETRLQLEAVNITDLSEN
RKQDKRFAFIRSDSGPTTSFESAACPGWFLCTAMEADQPVSLTNMPDEG
VMVTKFYFQEDE.

Human IL-1Ra, transcript 4, has the following mRNA sequence (NCBI Accession No. NM_{—}173843 and SEQ ID NO: 11):

gggcagctccaccctgggagggactgtggcccaggtactgcccgggtgctactttatgggcagcagctca
gttgagttagagtctggaagacctcagaagacctcctgtcctatgaggccctccccatggctttaggggg
attataaaactaatcatcaaagccaagaaggcaagagcaagcatgtaccgctgaaaacacaagataactg
cataagtaatgactttcagtgcagattcatagctaacccataaactgctggggcaaaaatcatcttggaa
ggctctgaacctcagaaaggattcacaagacgatctgccgaccctctgggagaaaatccagcaagATGca
agccttcagaatctgggatgttaaccagaagaccttctatctgaggaacaaccaactagttgctggatac
ttgcaaggaccaaatgtcaatttagaagaaaagatagatgtggtacccattgagcctcatgctctgttct
tgggaatccatggagggaagatgtgcctgtcctgtgtcaagtctggtgatgagaccagactccagctgga
ggcagttaacatcactgacctgagcgagaacagaaagcaggacaagcgcttcgccttcatccgctcagac
agcggccccaccaccagttttgagtctgccgcctgccccggttggttcctctgcacagcgatggaagctg
accagcccgtcagcctcaccaatatgcctgacgaaggcgtcatggtcaccaaattctacttccaggagga
cgagtagtactgcccaggcctgcctgttcccattcttgcatggcaaggactgcagggactgccagtcccc
ctgccccagggctcccggctatgggggcactgaggaccagccattgaggggtggaccctcagaaggcgtc
acaagaacctggtcacaggactctgcctcctcttcaactgaccagcctccatgctgcctccagaatggtc
tttctaatgtgtgaatcagagcacagcagcccctgcacaaagcccttccatgtcgcctctgcattcagga
tcaaaccccgaccacctgcccaacctgctctcctcttgccactgcctcttcctccctcattccaccttcc
catgccctggatccatcaggccacttgatgacccccaaccaagtggctcccacaccctgttttacaaaaa
agaaaagaccagtccatgagggaggtttttaagggtttgtggaaaatgaaaattaggatttcatgatttt
tttttttcagtccccgtgaaggagagcccttcatttggagattatgttctttcggggagaggctgaggac
ttaaaatattcctgcatttgtgaaatgatggtgaaagtaagtggtagcttttcccttctttttcttcttt
ttttgtgatgtcccaacttgtaaaaattaaaagttatggtactatgttagccccataattttttttttcc
ttttaaaacacttccataatctggactcctctgtccaggcactgctgcccagcctccaagctccatctcc
actccagattttttacagctgcctgcagtactttacctcctatcagaagtttctcagctcccaaggctct
gagcaaatgtggctcctgggggttctttcttcctctgctgaaggaataaattgctccttgacattgtaga
gcttctggcacttggagacttgtatgaaagatggctgtgcctctgcctgtctcccccaccgggctgggag
ctctgcagagcaggaaacatgactcgtatatgtctcaggtccctgcagggccaagcacctagcctcgctc
ttggcaggtactcagcgaatgaatgctgtatatgttgggtgcaaagttccctacttcctgtgacttcagc
tctgttttacaataaaatcttgaaaatgcctaaaaaaaaaaaaaaaaaaaaaaaaaaaaaaaaaaaaaaa
aaaaaaaaaaaaa.

Human IL-1Ra, transcript 4, encodes the following amino acid sequence (NCBI Accession No. NM_{—}173843 and SEQ ID NO: 12):

MQAFRIWDVNQKTFYLRNNQLVAGYLQGPNVNLEEKIDVVPIEPHALF
LGIHGGKMCLSCVKSGDETRLQLEAVNITDLSENRKQDKRFAFIRSDS
GPTTSFESAACPGWFLCTAMEADQPVSLTNMPDEGVMVTKFYFQEDE.

Human intracellular IL-1Ra, icIL-1Ra, is encoded by the following mRNA sequence (NCBI Accession No. M55646 and SEQ ID NO: 13):

agctccaccctgggagggactgtggcccaggtactgcccgggtgctactttatgggcagcagctcagttg
agttagagtctggaagacctcagaagacctcctgtcctatgaggccctccccATGgctttagagacgatc
tgccgaccctctgggagaaaatccagcaagatgcaagccttcagaatctgggatgttaaccagaagacct
tctatctgaggaacaaccaactagttgctggatacttgcaaggaccaaatgtcaatttagaagaaaagat
agatgtggtacccattgagcctcatgctctgttcttgggaatccatggagggaagatgtgcctgtcctgt
gtcaagtctggtgatgagaccagactccagctggaggcagttaacatcactgacctgagcgagaacagaa
agcaggacaagcgcttcgccttcatccgctcagacagtggccccaccaccagttttgagtctgccgcctg
ccccggttggttcctctgcacagcgatggaagctgaccagcccgtcagcctcaccaatatgcctgacgaa
ggcgtcatggtcaccaaattctacttccaggaggacgagtag.

Human intracellular IL-1Ra, icIL-1Ra, has the following amino acid sequence (NCBI Accession No. M55646 and SEQ ID NO: 14):

MALETICRPSGRKSSKMQAFRIWDVNQKTFYLRNNQLVAGYLQGPNVNL
EEKIDVVPIEPHALFLGIHGGKMCLSCVKSGDETRLQLEAVNITDLSEN
RKQDKRFAFIRSDSGPTTSFESAACPGWFLCTAMEADQPVSLTNMPDE
GVMVTKFYFQEDE.

### Human Recombinant IL-1Ra:

A recombinant form of human IL-1Ra (rHuIL-1Ra) was developed and tested in animal models for arthritis. This form of rHuIL-1Ra is also known as Anakinra or Kineret® differs from the native nonglycosylated IL-1Ra by the addition of an N-terminal methionine. It binds to IL-1R type I with the same affinity as IL-1β. Kineret® consists of 153 amino acids and has a molecular weight of 17.3 kilodaltons. It is produced by recombinant DNA technology using an E. coli bacterial expression system. Additional exemplary cytokine domain containing proteins that inhibit IL-1 inflammatory activity and bind to IL-1RI include receptor antagonists such as in U.S. Pat. No. 5,922,573, Evans et al. (1995), J. Biol. Chem. 270: 11477-11483; Greenfeder, et al. (1995) J. Biol. Chem. 270: 22460-22466, and Boraschi, et al. (1996) Frontiers in Bioscience 1:d270-308 (PubMed ID 9159234).

Anakinra has been investigated in several conditions considered mediated at least in part via IL-1. Some evidence suggests involvement of IL-1 in the pathogenesis of rheumatoid arthritis and septic shock (Jiang, Y. et al. 2000. Arthritis Rheum. 43:1001-1009; Fisher, C. J. et a1.1994. JAMA. 271:1836-1843; Okusawa, S. et al. 1988. J Clin Invest. 81:1162-1172; Bresnihan, B. et al. 1998. Rheum Dis Clin North Am. 24(3):615-628; Dayer, J. M. et al. 2001. Curr Opin Rheumatol. 13:170-176; Edwards, C. K. 2001. J Clin Rheumatol. 7:S17-S24). Anakinra has been approved by the FDA for the reduction in signs and symptoms of moderately to severely active rheumatoid arthritis in patients 18 years of age or older who have failed one or more disease-modifying antirheumatic drugs. Considering its high safety profile, administration of Anakinra has also been used in the treatment of arthritis in patients with Juvenile rheumatoid arthritis (Reiff, A. 2005. Curr Rheumatol Rep. 7:434-40). Other indications include prevention of graft-versus-host disease (GVHD) after bone marrow transplantation (Antin, J. H. et al. 1994. Blood. 84:1342-8), uveitis (Teoh, S. C. et al. 2007. Br J. Opthalmol. 91: 263-4) osteoarthritis (Caron, J. P. et al. 1996. Arthritis Rheum. 39:1535-44), asthma, inflammatory bowel disease, acute pancreatitis (Hynninen, M. et al. 1999. J Crit. Care. 14:63-8), psoriasis, and type II diabetes mellitus (Larsen, C. M. et al. 2007. N Engl J. Med. 356:1517-26). The systemic safety profile of IL-1Ra is extremely favorable, especially in comparison to other immunosuppressive treatments such as TNF-α blockers, cytotoxic agents, or even steroids.

Topical human recombinant IL-1Ra has been successfully used for prevention of corneal transplant rejection (Yamada, J. et al. 2000. Invest Opthalmol Vis Sci. 41:4203-8) and allergic conjunctivitis (Keane-Myers, A. M. et al. 1999. Invest Opthalmol Vis Sci. 40:3041-6) in experimental animal models. Similarly, using topical IL-1Ra significantly decreases corneal inflammation and leads to enhanced corneal transparency in the rat model of corneal alkali injury (Yamada, J. et al. 2003. Exp Eye Res. 76:161-7).

A recombinant form of human IL-1Ra (rHuIL-1Ra) was developed and approved for use in humans by subcutaneous injection for the treatment of arthritis. This form of rHuIL-1Ra, also known as Anakinra or Kineret® (Amgen Inc.), differs from the native nonglycosylated IL-1Ra by the addition of an N-terminal methionine. It binds to human IL-1R, type 1, (IL-1R1) with the same affinity as IL-1 β. Kineret® consists of 153 amino acids (see SEQ ID NO: 16) and has a molecular weight of 17.3 kilodaltons. It is produced by recombinant DNA technology using an E. coli bacterial expression system.

Exemplary compositions of the invention comprise one or more regions of SEQ ID NO: 16 or encoded by SEQ ID NO:15. Furthermore, compositions of the invention comprise the entire sequence of either SEQ ID NO: 15 or SEQ ID NO: 16. Anakinra/Kineret® is encoded by the following mRNA sequence (NCBI Accession No. M55646 and SEQ ID NO: 15):

agctccaccctgggagggactgtggcccaggtactgcccgggtgctactttatgggcagcagctcagttg
agttagagtctggaagacctcagaagacctcctgtcctatgaggccctccccATGgctttagagacgatc
tgccgaccctctgggagaaaatccagcaagatgcaagccttcagaatctgggatgttaaccagaagacct
tctatctgaggaacaaccaactagttgctggatacttgcaaggaccaaatgtcaatttagaagaaaagat
agatgtggtacccattgagcctcatgctctgttcttgggaatccatggagggaagatgtgcctgtcctgt
gtcaagtctggtgatgagaccagactccagctggaggcagttaacatcactgacctgagcgagaacagaa
agcaggacaagcgcttcgccttcatccgctcagacagtggccccaccaccagttttgagtctgccgcctg
ccccggttggttcctctgcacagcgatggaagctgaccagcccgtcagcctcaccaatatgcctgacgaa
ggcgtcatggtcaccaaattctacttccaggaggacgagtag.

Anakinra/Kineret® includes the following polypeptide sequence (DrugBank Accession No. BTD00060 and SEQ ID NO: 16):

MRPSGRKSSKMQAFRIWDVNQKTFYLRNNQLVAGYLQGPNVNLEEKID
VVPIEPHALFLGIHGGKMCLSCVKSGDETRLQLEAVNITDLSENRKQDK
RFAFIRSDSGPTTSFESAACPGWFLCTAMEADQPVSLTNMPDEGVMV
TKFYFQEDE

Exemplary proteins that inhibit IL-1 inflammatory activity and bind to IL-1RI can have an amino acid sequence at least 80, 85, 90, 95, 98% identical to anakinra or to other proteins disclosed or referenced herein.

### IL-1 Receptors:

The composition of the present invention comprises a polynucleotide, a polypeptide, an antibody, a compound, or a small molecule with means to inhibit the transcription, transcript stability, translation, modification, localization, secretion, or function of a polynucleotide or polypeptide encoding the IL-1 receptor, either type 1 or 2. In the present application the IL-1 Receptor, type 1 (IL-1R1), is defined by the polynucleotide sequence of SEQ ID NO: 17 or the polypeptide sequence of SEQ ID NO: 18. In the present application the IL-1 Receptor, type 2 (IL-1R2), transcript variants 1 and 2, are defined by the polynucleotide sequences of SEQ ID NO: 19 and 20, or the polypeptide sequence of SEQ ID NO: 21. IL-1R2 can function as a “decoy” receptor which binds IL-1 cytokines and inhibits IL-1R1. Polynucleotide or polypeptide compositions bind to one or more region(s) of IL-1R1 or IL-1R2, and associated isoforms, comprised by SEQ ID NO: 17-21.

IL-1R1 is encoded by the following mRNA sequence (NCBI Accession No.
NM_000877 and SEQ ID NO: 17):
tagacgcaccctctgaagatggtgactccctcctgagaagctggaccccttggtaaaagacaaggccttc
tccaagaagaatATGaaagtgttactcagacttatttgtttcatagctctactgatttcttctctggagg
ctgataaatgcaaggaacgtgaagaaaaaataattttagtgtcatctgcaaatgaaattgatgttcgtcc
ctgtcctcttaacccaaatgaacacaaaggcactataacttggtataaagatgacagcaagacacctgta
tctacagaacaagcctccaggattcatcaacacaaagagaaactttggtttgttcctgctaaggtggagg
attcaggacattactattgcgtggtaagaaattcatcttactgcctcagaattaaaataagtgcaaaatt
tgtggagaatgagcctaacttatgttataatgcacaagccatatttaagcagaaactacccgttgcagga
gacggaggacttgtgtgcccttatatggagttttttaaaaatgaaaataatgagttacctaaattacagt
ggtataaggattgcaaacctctacttcttgacaatatacactttagtggagtcaaagataggctcatcgt
gatgaatgtggctgaaaagcatagagggaactatacttgtcatgcatcctacacatacttgggcaagcaa
tatcctattacccgggtaatagaatttattactctagaggaaaacaaacccacaaggcctgtgattgtga
gcccagctaatgagacaatggaagtagacttgggatcccagatacaattgatctgtaatgtcaccggcca
gttgagtgacattgcttactggaagtggaatgggtcagtaattgatgaagatgacccagtgctaggggaa
gactattacagtgtggaaaatcctgcaaacaaaagaaggagtaccctcatcacagtgcttaatatatcgg
aaattgaaagtagattttataaacatccatttacctgttttgccaagaatacacatggtatagatgcagc
atatatccagttaatatatccagtcactaatttccagaagcacatgattggtatatgtgtcacgttgaca
gtcataattgtgtgttctgttttcatctataaaatcttcaagattgacattgtgctttggtacagggatt
cctgctatgattttctcccaataaaagcttcagatggaaagacctatgacgcatatatactgtatccaaa
gactgttggggaagggtctacctctgactgtgatatttttgtgtttaaagtcttgcctgaggtcttggaa
aaacagtgtggatataagctgttcatttatggaagggatgactacgttggggaagacattgttgaggtca
ttaatgaaaacgtaaagaaaagcagaagactgattatcattttagtcagagaaacatcaggcttcagctg
gctgggtggttcatctgaagagcaaatagccatgtataatgctcttgttcaggatggaattaaagttgtc
ctgcttgagctggagaaaatccaagactatgagaaaatgccagaatcgattaaattcattaagcagaaac
atggggctatccgctggtcaggggactttacacagggaccacagtctgcaaagacaaggttctggaagaa
tgtcaggtaccacatgccagtccagcgacggtcaccttcatctaaacaccagttactgtcaccagccact
aaggagaaactgcaaagagaggctcacgtgcctctcgggtagcatggagaagttgccaagagttctttag
gtgcctcctgtcttatggcgttgcaggccaggttatgcctcatgctgacttgcagagttcatggaatgta
actatatcatcctttatccctgaggtcacctggaatcagattattaagggaataagccatgacgtcaata
gcagcccagggcacttcagagtagagggcttgggaagatcttttaaaaaggcagtaggcccggtgtggtg
gctcacgcctataatcccagcactttgggaggctgaagtgggtggatcaccagaggtcaggagttcgaga
ccagcccagccaacatggcaaaaccccatctctactaaaaatacaaaaatgagctaggcatggtggcaca
cgcctgtaatcccagctacacctgaggctgaggcaggagaattgcttgaaccggggagacggaggttgca
gtgagccgagtttgggccactgcactctagcctggcaacagagcaagactccgtctcaaaaaaagggcaa
taaatgccctctctgaatgtttgaactgccaagaaaaggcatggagacagcgaactagaagaaagggcaa
gaaggaaatagccaccgtctacagatggcttagttaagtcatccacagcccaagggcggggctatgcctt
gtctggggaccctgtagagtcactgaccctggagcggctctcctgagaggtgctgcaggcaaagtgagac
tgacacctcactgaggaagggagacatattcttggagaactttccatctgcttgtattttccatacacat
ccccagccagaagttagtgtccgaagaccgaattttattttacagagcttgaaaactcacttcaatgaac
aaagggattctccaggattccaaagttttgaagtcatcttagctttccacaggagggagagaacttaaaa
aagcaacagtagcagggaattgatccacttcttaatgctttcctccctggcatgaccatcctgtcctttg
ttattatcctgcattttacgtctttggaggaacagctccctagtggcttcctccgtctgcaatgtccctt
gcacagcccacacatgaaccatccttcccatgatgccgctcttctgtcatcccgctcctgctgaaacacc
tcccaggggctccacctgttcaggagctgaagcccatgctttcccaccagcatgtcactcccagaccacc
tccctgccctgtcctccagcttcccctcgctgtcctgctgtgtgaattcccaggttggcctggtggccat
gtcgcctgcccccagcactcctctgtctctgctcttgcctcgacccttcctcctcctttgcctaggaggc
cttctcgcattttctctagctgatcagaattttaccaaaattcagaacatcctccaattccacagtctct
gggagactttccctaagaggcgacttcctctccagccttctctctctggtcaggcccactgcagagatgg
tggtgagcacatctgggaggctggtctccctccagctggaattgctgctctctgagggagaggctgtggt
ggctgtctctgtccctcactgccttccaggagcaatttgcacatgtaacatagatttatgtaatgcttta
tgtttaaaaacattccccaattatcttatttaatttttgcaattattctaattttatatatagagaaagt
gacctattttttaaaaaaatcacactctaagttctattgaacctaggacttgagcctccatttctggctt
ctagtctggtgttctgagtacttgatttcaggtcaataacggtcccccctcactccacactggcacgttt
gtgagaagaaatgacattttgctaggaagtgaccgagtctaggaatgcttttattcaagacaccaaattc
caaacttctaaatgttggaattttcaaaaattgtgtttagattttatgaaaaactcttctactttcatct
attctttccctagaggcaaacatttcttaaaatgtttcattttcattaaaaatgaaagccaaatttatat
gccaccgattgcaggacacaagcacagttttaagagttgtatgaacatggagaggacttttggtttttat
atttctcgtatttaatatgggtgaacaccaacttttatttggaataataattttcctcctaaacaaaaac
acattgagtttaagtctctgactcttgcctttccacctgctttctcctgggcccgctttgcctgcttgaa
ggaacagtgctgttctggagctgctgttccaacagacagggcctagctttcatttgacacacagactaca
gccagaagcccatggagcagggatgtcacgtcttgaaaagcctattagatgttttacaaatttaattttg
cagattattttagtctgtcatccagaaaatgtgtcagcatgcatagtgctaagaaagcaagccaatttgg
aaacttaggttagtgacaaaattggccagagagtgggggtgatgatgaccaagaattacaagtagaatgg
cagctggaatttaaggagggacaagaatcaatggataagcgtgggtggaggaagatccaaacagaaaagt
gcaaagttattccccatcttccaagggttgaattctggaggaagaagacacattcctagttccccgtgaa
cttcctttgacttattgtccccactaaaacaaaacaaaaaacttttaatgccttccacattaattagatt
ttcttgcagtttttttatggcatttttttaaagatgccctaagtgttgaagaagagtttgcaaatgcaac
aaaatatttaattaccggttgttaaaactggtttagcacaatttatattttccctctcttgcctttctta
tttgcaataaaaggtattgagccattttttaaatgacatttttgataaattatgtttgtactagttgatg
aaggagttttttttaacctgtttatataattttgcagcagaagccaaattttttgtatattaaagcacca
aattcatgtacagcatgcatcacggatcaatagactgtacttattttccaataaaattttcaaactttgt
actgttaaa.

IL-1R1 has the following amino acid sequence (NCBI Accession No. NM_{—}000877 and SEQ ID NO: 18):

MKVLLRLICFIALLISSLEADKCKEREEKIILVSSANEIDVRPCPLNPNEHKGTITWYKDDS
KTPVSTEQASRIHQHKEKLWFVPAKVEDSGHYYCVVRNSSYCLRIKISAKFVENEPNLC
YNAQAIFKQKLPVAGDGGLVCPYMEFFKNENNELPKLQWYKDCKPLLLDNIHFSGVKD
RLIVMNVAEKHRGNYTCHASYTYLGKQYPITRVIEFITLEENKPTRPVIVSPANETMEVD
LGSQIQLICNVTGQLSDIAYWKWNGSVIDEDDPVLGEDYYSVENPANKRRSTLITVLNIS
EIESRFYKHPFTCFAKNTHGIDAAYIQLIYPVTNFQKHMIGICVTLTVIIVCSVFIYKIFKIDI
VLWYRDSCYDFLPIKASDGKTYDAYILYPKTVGEGSTSDCDIFVFKVLPEVLEKQCGYK
LFIYGRDDYVGEDIVEVINENVKKSRRLIIILVRETSGFSWLGGSSEEQIAMYNALVQDGI
KVVLLELEKIQDYEKMPESIKFIKQKHGAIRWSGDFTQGPQSAKTRFWKNVRYHMPVQ
RRSPSSKHQLLSPATKEKLQREAHVPLG.

IL-1R2, transcript variant 1, is encoded by the following mRNA sequence (NCBI Accession No. NM_{—}004633 and SEQ ID NO: 19):

cccgtgaggaggaaaaggtgtgtccgctgccacccagtgtgagcgggtgacaccacccggttaggaaatc
ccagctcccaagagggtataaatccctgctttactgctgagctcctgctggaggtgaaagtctggcctgg
cagccttccccaggtgagcagcaacaaggccacgtgctgctgggtctcagtcctccacttcccgtgtcct
ctggaagttgtcaggagcaATGttgcgcttgtacgtgttggtaatgggagtttctgccttcacccttcag
cctgcggcacacacaggggctgccagaagctgccggtttcgtgggaggcattacaagcgggagttcaggc
tggaaggggagcctgtagccctgaggtgcccccaggtgccctactggttgtgggcctctgtcagcccccg
catcaacctgacatggcataaaaatgactctgctaggacggtcccaggagaagaagagacacggatgtgg
gcccaggacggtgctctgtggcttctgccagccttgcaggaggactctggcacctacgtctgcactacta
gaaatgcttcttactgtgacaaaatgtccattgagctcagagtttttgagaatacagatgctttcctgcc
gttcatctcatacccgcaaattttaaccttgtcaacctctggggtattagtatgccctgacctgagtgaa
ttcacccgtgacaaaactgacgtgaagattcaatggtacaaggattctcttcttttggataaagacaatg
agaaatttctaagtgtgagggggaccactcacttactcgtacacgatgtggccctggaagatgctggcta
ttaccgctgtgtcctgacatttgcccatgaaggccagcaatacaacatcactaggagtattgagctacgc
atcaagaaaaaaaaagaagagaccattcctgtgatcatttcccccctcaagaccatatcagcttctctgg
ggtcaagactgacaatcccgtgtaaggtgtttctgggaaccggcacacccttaaccaccatgctgtggtg
gacggccaatgacacccacatagagagcgcctacccgggaggccgcgtgaccgaggggccacgccaggaa
tattcagaaaataatgagaactacattgaagtgccattgatttttgatcctgtcacaagagaggatttgc
acatggattttaaatgtgttgtccataataccctgagttttcagacactacgcaccacagtcaaggaagc
ctcctccacgttctcctggggcattgtgctggccccactttcactggccttcttggttttggggggaata
tggatgcacagacggtgcaaacacagaactggaaaagcagatggtctgactgtgctatggcctcatcatc
aagactttcaatcctatcccaagtgaaataaatggaatgaaataattcaaacacaaaaaaaaaaaaaaaa
aaaaaaaaaaaaaa.

IL-1R2, transcript variant 2, is encoded by the following mRNA sequence (NCBI Accession No. NM_{—}173343 and SEQ ID NO: 20):

gggatgggagatactgttgtggtcacctctggaaaatacattctgctactcttaaaaactagtgacgctc
atacaaatcaacagaaagagcttctgaaggaagactttaaagctgcttctgccacgtgctgctgggtctc
agtcctccacttcccgtgtcctctggaagttgtcaggagcaATGttgcgcttgtacgtgttggtaatggg
agtttctgccttcacccttcagcctgcggcacacacaggggctgccagaagctgccggtttcgtgggagg
cattacaagcgggagttcaggctggaaggggagcctgtagccctgaggtgcccccaggtgccctactggt
tgtgggcctctgtcagcccccgcatcaacctgacatggcataaaaatgactctgctaggacggtcccagg
agaagaagagacacggatgtgggcccaggacggtgctctgtggcttctgccagccttgcaggaggactct
ggcacctacgtctgcactactagaaatgcttcttactgtgacaaaatgtccattgagctcagagtttttg
agaatacagatgctttcctgccgttcatctcatacccgcaaattttaaccttgtcaacctctggggtatt
agtatgccctgacctgagtgaattcacccgtgacaaaactgacgtgaagattcaatggtacaaggattct
cttcttttggataaagacaatgagaaatttctaagtgtgagggggaccactcacttactcgtacacgatg
tggccctggaagatgctggctattaccgctgtgtcctgacatttgcccatgaaggccagcaatacaacat
cactaggagtattgagctacgcatcaagaaaaaaaaagaagagaccattcctgtgatcatttcccccctc
aagaccatatcagcttctctggggtcaagactgacaatcccgtgtaaggtgtttctgggaaccggcacac
ccttaaccaccatgctgtggtggacggccaatgacacccacatagagagcgcctacccgggaggccgcgt
gaccgaggggccacgccaggaatattcagaaaataatgagaactacattgaagtgccattgatttttgat
cctgtcacaagagaggatttgcacatggattttaaatgtgttgtccataataccctgagttttcagacac
tacgcaccacagtcaaggaagcctcctccacgttctcctggggcattgtgctggccccactttcactggc
cttcttggttttggggggaatatggatgcacagacggtgcaaacacagaactggaaaagcagatggtctg
actgtgctatggcctcatcatcaagactttcaatcctatcccaagtgaaataaatggaatgaaataattc
aaacacaaaaaaaaaaaaaaaaaaaaaaaaaaaaaa.

IL-1R2, transcript variants 1 and 2, have the following amino acid sequence (NCBI Accession No. NM_{—}004633, NM_{—}173343, and SEQ ID NO: 21):

MLRLYVLVMGVSAFTLQPAAHTGAARSCRFRGRHYKREFRLEGEPVALRC
PQVPYWLWASVSPRINLTWHKNDSARTVPGEEETRMWAQDGALWLLPAL
QEDSGTYVCTTRNASYCDKMSIELRVFENTDAFLPFISYPQILTLSTSGV
LVCPDLSEFTRDKTDVKIQWYKDSLLLDKDNEKFLSVRGTTHLLVHDVA
LEDAGYYRCVLTFAHEGQQYNITRSIELRIKKKKEETIPVIISPLKTISA
SLGSRLTIPCKVFLGTGTPLTTMLWWTANDTHIESAYPGGRVTEGPRQE
YSENNENYIEVPLIFDPVTREDLHMDFKCVVHNTLSFQTLRTTVKEAS
STFSWGIVLAPLSLAFLVLGGIWMHRRCKHRTGKADGLTVLWPHHQD
FQSYPK.

### Interleukin-1 Receptor (Type 2) Antagonist (IL-1Ra3):

The present invention comprises compositions with means to inhibit or enhance the activity of the human IL-1R2. Compositions that comprise the IL-1R2 antagonist, IL-1Ra3, have either agonist or antagonist activity regarding the efficacy of IL-1R1 function. The composition comprises a polynucleotide, a polypeptide, an antibody, a compound, or a small molecule with means to inhibit the transcription, transcript stability, translation, modification, localization, secretion, or function of a polynucleotide or polypeptide encoding IL-1Ra3. The inhibitory polynucleotide or polypeptide composition binds to one or more region(s) of IL-1Ra3 comprised by SEQ ID NO: 22 and SEQ ID NO: 23.

IL-1Ra3 is encoded by a region or the entirety of the following mRNA sequence (NCBI Accession No. AF_{—}057168 and SEQ ID NO: 22): (for this sequence, the bolded and capitalized codon does not encode methionine, but rather represents the codon that encodes the first amino acid of the corresponding polypeptide)

cagaagacctcctgtcctatgaggccctccccatggctttaggtaagctccttccactctcattttttca
cctgagaaatgagagaggaaaatgtctacaattggtgtttatcaaatgctttcaggctctggtgagcaag
cgtccaggaaaatgtcaagcgcatggagctccaggcctgtctgggggatctgggcacggggaggcatcca
tgggagaccatgcaggcactctgaggcaggggctgcaagctagtgcctgctggggcagcaggtgaacaga
gaggtgtaactgctgtgacagaagtcatggagtccttggagtgtgagggtcattttccactgttgataga
atagggaaattggtgaaatagccctgttaaatgagagaaagaacagtgtgagctcaatgagaaatactaa
tagaatgtggcactgagccacaaggtctgagggttgattgataaggaagggtggggactgtggagaatta
agggcttggcacaggtcagttccaccagttgtcacaagagaatgcaggctcaggtggccagaacttctcg
cttttccagaagagtccgatattctgatttcattatatatagtattctgattaaaccagacaataaagca
agcagataaaatatttaaagtataagctgccagtttgcaacctccggttaggatttgtgtggggcaaaga
aaaaaactctcaggatcattggtatgtagactctaattttaagtttctaatttaaaattggcccctgagg
ctgggcgtggtggctcacacctgtaatcccagcattttgggaggccaaggtgggtggatctcttgaggtc
aagagttcaaggcctgcctggccaacatggtgaaaccctgtctctattaaaaatacaaaaattagctggg
catggtggtgcatgtctgcaatcttagctacttgggtagctaaggcaggagaattgctggaacccgggag
gtagaggttgcagtgaatggagatcacaccactgcactccagtctgggcaatagagagagacgctctctc
taaaaaaaaatatgtaaagataaataaaatgaaataaaataggcctctaatgagcaggccattctccttt
ctgggtcttactttccttgcactcctttctgggtgttaagaggaggtctagaggaagctggacaactctt
agcttgtagtaagcacagtggaagtatcagctcttaatgggtcatggacacgttacgaagctaggcgccg
tgctgagcactttacatggtttatcccactgaaccctctcaataaccctatgaggaagggctattattgc
tcacattttcagaagaggaaatggatatagagagattagataatttgcccatggccagacagctagtata
agaggaggaggtggattgactgcagacattctgtcttcaaaccactacactatgctatggaggcacagag
acttaatgaaatcatggagaggggaattgctttgtcaaccacaagcagttattccgggggcagcagatcc
tcccctgtcccccagtggtacaatggtccctggtgggttgtgctacaatgttagcccatggtcttatgtg
tttttcaaatgtgtaaagtaggatgctggaaccactcttagaaccagataccaatacattgtgaagaaat
aaatctctgtgcttaaaactggttcatcccaaaatattttgaactgacacacaataggtgctaaataaat
gtgtgttaacttgaattggattgaattcgggaaaaaagtgcaataagcttagtgaagacaccatgttccc
tgggtagaggaaccacattctccatctaaggccaggagtatgggaggtatcaatgtttgcccagcacaga
acagggtgccaagaagagaaaagttgacggggtgcatactctgactggaaactggaagggtgagaacaga
gggtaaaggatagagatggaaccatgtgcatacactttgtgttaccttggacaagtcattcatttctctg
gacctctgctttctctctacacaatggggtcccaccacttcccttacagctgacttgtatgaagaaggag
gtggaggaggaggagaaggtgaagacaatgCTGactcaaagggtaaattatttttaggatccaagtttga
aaacaattttaggctactagatatgaacaacatcttgattatgtagttgaaggaaattaaagatgaatgg
tttaattaaaaattaatcagaatgaaaacgattgattactaatatatctgcaatggtttattttcctgag
tggcagactcactaaggtttttgaatactcctgtgtgattgctctatgtatgtatgtatgtatgtatgta
tgcatgtatctatctatctgttgtctaatagaatggatcacatctctgctaataaaaacactacactggc
agggtacaattataatcattaactgtgcctggaatttgcagcagcagccaccagaggtaccagtgccctt
taagggttcataatttagaataatccaattatctgagtttttcagggactgaggggtttggcaaggtgta
gaactttcagtaataaagtcaagaaagtcctggacaaaccaaggtagttggtcactctagtccataacca
ggtaaagagctttccctgtaacctgtgtaaggttttagaatcatttctttccttattaccaaaaatcctc
cccaaattttcaagaaattatgaactaaatagttactctatgagataggagttcagcccaaaagaaacac
cataagaacaaatataattcttgcttatgttaaccatgcaatgaagcagagagaaaaagtcagtggcctc
tttaggaggactgtagtgtgggaagaaataactaaactgggtttcaatcctggcctggccaggatctgga
gcaagtgagttaatctttctaagccttgagtagtttataaaagaatggccactccatagacagagtagcc
tgaaccttgagttcttctataaagtcactatgaatttatactcattttgaaagtgggtgtcaatatgtct
gtccactttgcacagctgttatgtggacaaaaggagatctgtgtgaaagtgtaacacagagcctaaacta
taacaggtaagcaacacagttgtccct.

One or more isoforms of IL-1Ra3 comprise the following amino acid sequence (NCBI Accession No. AF_{—}057168 and SEQ ID NO: 23):

DLYEEGGGGGGEGEDNADSK.

### Interleukin-1 Receptor Accessory Protein (IL-1 RAP):

Compositions that inhibit the activity of human IL-1RAP inhibit IL-1RAP binding to an IL-1 cytokine or an IL-1 receptor, and subsequent transduction of downstream intracellular signals. Compositions that comprise an inhibitor of IL-1RAP function antagonize the activity of an IL-1 receptor. The composition comprises a polynucleotide, a polypeptide, an antibody, a compound, or a small molecule with means to inhibit the transcription, transcript stability, translation, modification, localization, secretion, or function of a polynucleotide or polypeptide encoding IL-1RAP. The inhibitory polynucleotide or polypeptide composition binds to one or more region(s) of IL-1RAP, and associated isoforms, comprised by SEQ ID NO: 24-27. IL-1RAP, transcript variant 1, is encoded by the following mRNA sequence (NCBI Accession No. NM_{—}002182 and SEQ ID NO: 24):

tgccgggatccaggtctccggggtccgctttggccagaggcgcggaaggaagcagtgcccggcgacactg
cacccatcccggctgcttttgctgcgccctctcagcttcccaagaaaggcatcgtcatgtgatcatcacc
taagaactagaacatcagcaggccctagaagcctcactcttgcccctccctttaatatctcaaaggATGa
cacttctgtggtgtgtagtgagtctctacttttatggaatcctgcaaagtgatgcctcagaacgctgcga
tgactggggactagacaccatgaggcaaatccaagtgtttgaagatgagccagctcgcatcaagtgccca
ctctttgaacacttcttgaaattcaactacagcacagcccattcagctggccttactctgatctggtatt
ggactaggcaggaccgggaccttgaggagccaattaacttccgcctccccgagaaccgcattagtaagga
gaaagatgtgctgtggttccggcccactctcctcaatgacactggcaactatacctgcatgttaaggaac
actacatattgcagcaaagttgcatttcccttggaagttgttcaaaaagacagctgtttcaattccccca
tgaaactcccagtgcataaactgtatatagaatatggcattcagaggatcacttgtccaaatgtagatgg
atattttccttccagtgtcaaaccgactatcacttggtatatgggctgttataaaatacagaattttaat
aatgtaatacccgaaggtatgaacttgagtttcctcattgccttaatttcaaataatggaaattacacat
gtgttgttacatatccagaaaatggacgtacgtttcatctcaccaggactctgactgtaaaggtagtagg
ctctccaaaaaatgcagtgccccctgtgatccattcacctaatgatcatgtggtctatgagaaagaacca
ggagaggagctactcattccctgtacggtctattttagttttctgatggattctcgcaatgaggtttggt
ggaccattgatggaaaaaaacctgatgacatcactattgatgtcaccattaacgaaagtataagtcatag
tagaacagaagatgaaacaagaactcagattttgagcatcaagaaagttacctctgaggatctcaagcgc
agctatgtctgtcatgctagaagtgccaaaggcgaagttccaaagcagccaaggtgaagcagaaagtgcc
agctccaagatacacagtggaactggcttgtggttttggagccacagtcctgctagtggtgattctcatt
gttgtttaccatgtttactggctagagatggtcctattttaccgggctcattttggaacagatgaaacca
ttttagatggaaaagagtatgatatttatgtatcctatgcaaggaatgcggaagaagaagaatttgtatt
actgaccctccgtggagttttggagaatgaatttggatacaagctgtgcatctttgaccgagacagtctg
cctgggggaattgtcacagatgagactttgagcttcattcagaaaagcagacgcctcctggttgttctaa
gccccaactacgtgctccagggaacccaagccctcctggagctcaaggctggcctagaaaatatggcctc
tcggggcaacatcaacgtcattttagtacagtacaaagctgtgaaggaaacgaaggtgaaagagctgaag
agggctaagacggtgctcacggtcattaaatggaaaggggaaaaatccaagtatccacagggcaggttct
ggaagcagctgcaggtggccatgccagtgaagaaaagtcccaggcggtctagcagtgatgagcagggcct
ctcgtattcatctttgaaaaatgtatgaaaggaataatgaaaagggtaaaaagaacaaggggtgctccag
gaagaaagagtccccccagtcttcattcgcagtttatggtttcataggcaaaaataatggtctaagcctc
ccaatagggataaatttagggtgactgtgtggctgactattctgcttcctcaggcaacactaaagtttag
aaagatatcatcaacgttctgtcaccagtctctgatgccactatgttctttgcaggcaaagacttgttca
atgcgaatttccccttctacattgtctatccctgtttttatatgtctccattctttttaaaatcttaaca
tatggagcagcctttcctatgaatttaaatatgcctttaaaataagtcactgttgacagggtcatgagtt
tccgagtatagttttctttttatcttatttttactcgtccgttgaaaagataatcaaggcctacatttta
gctgaggataatgaacttttttcctcattcggctgtataatacataaccacagcaagactgacatccact
taggatgatacaaagcagtgtaactgaaaatgtttcttttaattgatttaaaggacttgtcttctatacc
acccttgtcctcatctcaggtaatttatgaaatctatgtaaacttgaaaaatatttcttaatttttgttt
ttgctccagtcaattcctgattatccacaggtcaacccacattttttcattccttctccctatctgctta
tatcgcattgctcatttagagtttgcaggaggctccatactaggttcagtctgaaagaaatctcctaatg
gtgctatagagagggaggtaacagaaagactcttttagggcatttttctgactcatgaaaagagcacaga
aaaggatgtttggcaatttgtcttttaagtcttaaccttgctaatgtgaatactgggaaagtgatttttt
ctcactcgtttttgttgctccattgtaaagggcggaggtcagtcttagtggccttgagagttgcttttgg
cattaatattctaagagaattaactgtatttcctgtcacctattcactagtgcaggaaatatacttgctc
caaataagtcagtatgagaagtcactgtcaatgaaagttgttttgtttgttttcagtaatattttgctgt
ttttaagacttggaaaactaagtgcagagtttacagagtggtaaatatctatgttacatgtagattatac
atatatatacacacgtgtatatgagatatatatcttatatctccacaaacacaaattatatatatacata
tccacacacatacattacatatatctgtgtatataaatccacatgcacatgaaatatatatatatatata
atttgtgtgtgtgtatgtgtatgtatatgactttaaatagctatgggtacaatattaaaaaccactggaa
ctcttgtccagtttttaaattatgtttttactggaatgtttttgtgtcagtgttttctgtacatattatt
tgttaattcacagctcacagagtgatagttgtcatagttcttgccttccctaagtttatataaataactt
aagtattgctacagtttatctaggttgcagtggcatctgctgtgcacagagcttccatggtcactgctaa
gcagtagccagccatcgggcattaattgatttcctactatattcccagcagacacatttagaaactaagc
tatgttaacctcagtgctcaactatttgaactgttgagtgataaaggaaacaaatataactgtaaatgaa
tcttggtatcctgtgaaacagaataattcgtaatttaagaaagcccttatcccggtaacatgaatgttga
tgaacaaatgtaaaattatatcctatatttaagtacccataataaatcatttccctctataagtgttatt
gattattttaaattgaaaaaagtttcacttggatgaaaaaagtagaaaagtaggtcattcttggatctac
ttttttttagccttattaatatttttccctattagaaaccacaattactccctctattaacccttcactt
actagaccagaaaagaacttattccagataagctttgaatatcaattcttacataaactttaggcaaaca
gggaatagtctagtcaccaaaggaccattctcttgccaatgctgcattccttttgcacttttggattcca
tatttatcccaaatgctgttgggcacccctagaaataccttgatgttttttctatttatatgcctgcctt
tggtacttaattttacaaatgctgtaatataaagcatatcaagtttatgtgatacgtatcattgcaagag
aatttgtttcaagatttttttttaatgttccagaagatggccaatagagaacattcaagggaaatgggga
aacataatttagagaacaagaacaaaccatgtctcaaatttttttaaaaaaaattaatggttttaaatat
atgctatagggacgttccatgcccaggttaacaaagaactgtgatatatagagtgtctaattacaaaatc
atatacgatttatttaattctcttctgtattgtaacttagatgattcccaaggactctaataaaaaatca
cttcattgtatttggaaacaaaaacatcattcattaattacttattttctttccataggttttaatattt
tgagagtgtcttttttatttcattcatgaacttttgtatttttcatttttcatttgatttgtaaatttac
ttatgttaaaaataaaccatttattttcagctttg.

IL-1RAP, transcript variant 1, has the following amino acid sequence (NCBI Accession No. NM_{—}002182 and SEQ ID NO: 25):

MTLLWCVVSLYFYGILQSDASERCDDWGLDTMRQIQVFEDEPARIKCPL
FEHFLKFNYSTAHSAGLTLIWYWTRQDRDLEEPINFRLPENRISKEKDV
LWFRPTLLNDTGNYTCMLRNTTYCSKVAFPLEVVQKDSCFNSPMKLPV
HKLYIEYGIQRITCPNVDGYFPSSVKPTITWYMGCYKIQNFNNVIPEGM
NLSFLIALISNNGNYTCVVTYPENGRTFHLTRTLTVKVVGSPKNAVPPV
IHSPNDHVVYEKEPGEELLIPCTVYFSFLMDSRNEVWWTIDGKKPDDI
TIDVTINESISHSRTEDETRTQILSIKKVTSEDLKRSYVCHARSAKGEVA
KAAKVKQKVPAPRYTVELACGFGATVLLVVILIVVYHVYWLEMVLFYR
AHFGTDETILDGKEYDIYVSYARNAEEEEFVLLTLRGVLENEFGYKLC
IFDRDSLPGGIVTDETLSFIQKSRRLLVVLSPNYVLQGTQALLELKAG
LENMASRGNINVILVQYKAVKETKVKELKRAKTVLTVIKWKGEKSKYP
QGRFWKQLQVAMPVKKSPRRSSSDEQGLSYSSLKNV.

IL-1RAP, transcript variant 2, is encoded by the following mRNA sequence (NCBI Accession No. NM_{—}134470 and SEQ ID NO: 26):

tgccgggatccaggtctccggggtccgctttggccagaggcgcggaaggaagcagtgcccggcgacactg
cacccatcccggctgcttttgctgcgccctctcagcttcccaagaaaggcatcgtcatgtgatcatcacc
taagaactagaacatcagcaggccctagaagcctcactcttgcccctccctttaatatctcaaaggATGa
cacttctgtggtgtgtagtgagtctctacttttatggaatcctgcaaagtgatgcctcagaacgctgcga
tgactggggactagacaccatgaggcaaatccaagtgtttgaagatgagccagctcgcatcaagtgccca
ctctttgaacacttcttgaaattcaactacagcacagcccattcagctggccttactctgatctggtatt
ggactaggcaggaccgggaccttgaggagccaattaacttccgcctccccgagaaccgcattagtaagga
gaaagatgtgctgtggttccggcccactctcctcaatgacactggcaactatacctgcatgttaaggaac
actacatattgcagcaaagttgcatttcccttggaagttgttcaaaaagacagctgtttcaattccccca
tgaaactcccagtgcataaactgtatatagaatatggcattcagaggatcacttgtccaaatgtagatgg
atattttccttccagtgtcaaaccgactatcacttggtatatgggctgttataaaatacagaattttaat
aatgtaatacccgaaggtatgaacttgagtttcctcattgccttaatttcaaataatggaaattacacat
gtgttgttacatatccagaaaatggacgtacgtttcatctcaccaggactctgactgtaaaggtagtagg
ctctccaaaaaatgcagtgccccctgtgatccattcacctaatgatcatgtggtctatgagaaagaacca
ggagaggagctactcattccctgtacggtctattttagttttctgatggattctcgcaatgaggtttggt
ggaccattgatggaaaaaaacctgatgacatcactattgatgtcaccattaacgaaagtataagtcatag
tagaacagaagatgaaacaagaactcagattttgagcatcaagaaagttacctctgaggatctcaagcgc
agctatgtctgtcatgctagaagtgccaaaggcgaagttgccaaagcagccaaggtgaagcagaaaggta
atagatgcggtcagtgatgaatctctcagctccaaattaacattgtggtgaataaggacaaaaggagaga
ttgagaacaagagagctccagcacctagcccgacggcatctaacccatagtaatgaatcaaacttaaatg
aaaaatatgaaagttttcatctatgtaagatactcaaaatattgtttctgatattgttagtaccgtaatg
cccaaatgtagctaaaaaaatcgacgtgagtacagtgagacacaattttgtgtctgtacaattatgaaaa
attaaaaacaaagaaaatattcaaagctaccaaagatagaaaaaactggtagagccacatattgttggtg
aattattaagacccttttaaaaatcattcatggtagagtttaagagtcataaaaaagattgcatcatctg
acctaagactttcggaatttttcctgaacaaataacagaaagggaattatataccttttaatattattag
aagcattatctgtagttgtaaaacattattaatagcagccatccaattgtatgcaactaattaaggtatt
gaatgtttattttccaaaaatgcataattataatattattttaaacactatgtatcaatatttaagcagg
tttataatataccagcagccacaattgctaaaatgaaaatcatttaaattatgattttaaatggtataaa
catgatttctatgttgatagtactatattattctacaataaatggaaattataaagccttcttgtcagaa
gtgctgctcctaaaaaaaaaaaaaaaaaaaaaa.

IL-1RAP, transcript variant 2, has the following amino acid sequence (NCBI Accession No. NM_{—}134470 and SEQ ID NO: 27):

MTLLWCVVSLYFYGILQSDASERCDDWGLDTMRQIQVFEDEPARIKCPL
FEHFLKFNYSTAHSAGLTLIWYWTRQDRDLEEPINFRLPENRISKEKDVL
WFRPTLLNDTGNYTCMLRNTTYCSKVAFPLEVVQKDSCFNSPMKLPVH
KLYIEYGIQRITCPNVDGYFPSSVKPTITWYMGCYKIQNFNNVIPEGMN
LSFLIALISNNGNYTCVVTYPENGRTFHLTRTLTVKVVGSPKNAVPPVIH
SPNDHVVYEKEPGEELLIPCTVYFSFLMDSRNEVWWTIDGKKPDDITI
DVTINESISHSRTEDETRTQILSIKKVTSEDLKRSYVCHARSAKGEVAK
AAKVKQKGNRCGQ.

### Interleukin-1 Receptor Associated Kinase 1 (IRAK1):

The invention also comprises compositions and methods to inhibit the activity of human IRAK1, defined as the ability of this protein to bind an IL-1 receptor following ligation of this receptor with IL-1, as well as to transduce downstream signals leading to an inflammatory response. Compositions that comprise an inhibitor of IRAK1 antagonize downstream signaling from an IL-1 receptor. The composition comprises a polynucleotide, a polypeptide, an antibody, a compound, or a small molecule with means to inhibit the transcription, transcript stability, translation, modification, localization, secretion, or function of a polynucleotide or polypeptide encoding IRAK1. The inhibitory polynucleotide or polypeptide composition binds to one or more region(s) of IRAK1, and associated isoforms, comprised by SEQ ID NO: 28-33. IRAK1, transcript variant 1, is encoded by the following mRNA sequence (NCBI Accession No. NM_{—}001569 and SEQ ID NO: 28):

cgcggacccggccggcccaggcccgcgcccgccgcggccctgagaggccccggcaggtcccggcccggcg
gcggcagccATGgccggggggccgggcccgggggagcccgcagcccccggcgcccagcacttcttgtacg
aggtgccgccctgggtcatgtgccgcttctacaaagtgatggacgccctggagcccgccgactggtgcca
gttcgccgccctgatcgtgcgcgaccagaccgagctgcggctgtgcgagcgctccgggcagcgcacggcc
agcgtcctgtggccctggatcaaccgcaacgcccgtgtggccgacctcgtgcacatcctcacgcacctgc
agctgctccgtgcgcgggacatcatcacagcctggcaccctcccgccccgcttccgtccccaggcaccac
tgccccgaggcccagcagcatccctgcacccgccgaggccgaggcctggagcccccggaagttgccatcc
tcagcctccaccttcctctccccagcttttccaggctcccagacccattcagggcctgagctcggcctgg
tcccaagccctgcttccctgtggcctccaccgccatctccagccccttcttctaccaagccaggcccaga
gagctcagtgtccctcctgcagggagcccgcccctttccgttttgctggcccctctgtgagatttcccgg
ggcacccacaacttctcggaggagctcaagatcggggagggtggctttgggtgcgtgtaccgggcggtga
tgaggaacacggtgtatgctgtgaagaggctgaaggagaacgctgacctggagtggactgcagtgaagca
gagcttcctgaccgaggtggagcagctgtccaggtttcgtcacccaaacattgtggactttgctggctac
tgtgctcagaacggcttctactgcctggtgtacggcttcctgcccaacggctccctggaggaccgtctcc
actgccagacccaggcctgcccacctctctcctggcctcagcgactggacatccttctgggtacagcccg
ggcaattcagtttctacatcaggacagccccagcctcatccatggagacatcaagagttccaacgtcctt
ctggatgagaggctgacacccaagctgggagactttggcctggcccggttcagccgctttgccgggtcca
gccccagccagagcagcatggtggcccggacacagacagtgcggggcaccctggcctacctgcccgagga
gtacatcaagacgggaaggctggctgtggacacggacaccttcagctttggggtggtagtgctagagacc
ttggctggtcagagggctgtgaagacgcacggtgccaggaccaagtatctgaaagacctggtggaagagg
aggctgaggaggctggagtggctttgagaagcacccagagcacactgcaagcaggtctggctgcagatgc
ctgggctgctcccatcgccatgcagatctacaagaagcacctggaccccaggcccgggccctgcccacct
gagctgggcctgggcctgggccagctggcctgctgctgcctgcaccgccgggccaaaaggaggcctccta
tgacccaggtgtacgagaggctagagaagctgcaggcagtggtggcgggggtgcccgggcattcggaggc
cgccagctgcatccccccttccccgcaggagaactcctacgtgtccagcactggcagagcccacagtggg
gctgctccatggcagcccctggcagcgccatcaggagccagtgcccaggcagcagagcagctgcagagag
gccccaaccagcccgtggagagtgacgagagcctaggcggcctctctgctgccctgcgctcctggcactt
gactccaagctgccctctggacccagcacccctcagggaggccggctgtcctcagggggacacggcagga
gaatcgagctgggggagtggcccaggatcccggcccacagccgtggaaggactggcccttggcagctctg
catcatcgtcgtcagagccaccgcagattatcatcaaccctgcccgacagaagatggtccagaagctggc
cctgtacgaggatggggccctggacagcctgcagctgctgtcgtccagctccctcccaggcttgggcctg
gaacaggacaggcaggggcccgaagaaagtgatgaatttcagagctgatgtgttcacctgggcagatccc
ccaaatccggaagtcaaagttctcatggtcagaagttctcatggtgcacgagtcctcagcactctgccgg
cagtgggggtgggggcccatgcccgcgggggagagaaggaggtggccctgctgttctaggctctgtgggc
ataggcaggcagagtggaaccctgcctccatgccagcatctgggggcaaggaaggctggcatcatccagt
gaggaggctggcgcatgttgggaggctgctggctgcacagacccgtgaggggaggagaggggctgctgtg
caggggtgtggagtagggagctggctcccctgagagccatgcagggcgtctgcagcccaggcctctggca
gcagctctttgcccatctctttggacagtggccaccctgcacaatggggccgacgaggcctagggccctc
ctacctgcttacaatttggaaaagtgtggccgggtgcggtggctcacgcctgtaatcccagcactttggg
aggccaaggcaggaggatcgctggagcccagtaggtcaagaccagccagggcaacatgatgagaccctgt
ctctgccaaaaaattttttaaactattagcctggcgtggtagcgcacgcctgtggtcccagctgctgggg
aggctgaagtaggaggatcatttatgcttgggaggtcgaggctgcagtgagtcatgattgtatgactgca
ctccagcctgggtgacagagcaagaccctgtttcaaaaagaaaaaccctgggaaaagtgaagtatggctg
taagtctcatggttcagtcctagcaagaagcgagaattctgagatcctccagaaagtcgagcagcaccca
cctccaacctcgggccagtgtcttcaggctttactggggacctgcgagctggcctaatgtggtggcctgc
aagccaggccatccctgggcgccacagacgagctccgagccaggtcaggcttcggaggccacaagctcag
cctcaggcccaggcactgattgtggcagaggggccactacccaaggtctagctaggcccaagacctagtt
acccagacagtgagaagcccctggaaggcagaaaagttgggagcatggcagacagggaagggaaacattt
tcagggaaaagacatgtatcacatgtcttcagaagcaagtcaggtttcatgtaaccgagtgtcctcttgc
gtgtccaaaagtagcccagggctgtagcacaggcttcacagtgattttgtgttcagccgtgagtcacact
acatgcccccgtgaagctgggcattggtgacgtccaggttgtccttgagtaataaaaacgtatgttgcaa
taaaaaaaaaaaaaaaaaa.

IRAK1, transcript variant 1, has the following amino acid sequence (NCBI Accession No. NM_{—}001569 and SEQ ID NO: 29):

MAGGPGPGEPAAPGAQHFLYEVPPWVMCRFYKVMDALEPADWCQFA
ALIVRDQTELRLCERSGQRTASVLWPWINRNARVADLVHILTHLQLLR
ARDIITAWHPPAPLPSPGTTAPRPSSIPAPAEAEAWSPRKLPSSASTF
LSPAFPGSQTHSGPELGLVPSPASLWPPPPSPAPSSTKPGPESSVSLL
QGARPFPFCWPLCEISRGTHNFSEELKIGEGGFGCVYRAVMRNTVYAV
KRLKENADLEWTAVKQSFLTEVEQLSRFRHPNIVDFAGYCAQNGFYCLV
YGFLPNGSLEDRLHCQTQACPPLSWPQRLDILLGTARAIQFLHQDSPSLI
HGDIKSSNVLLDERLTPKLGDFGLARFSRFAGSSPSQSSMVARTQTVRG
TLAYLPEEYIKTGRLAVDTDTFSFGVVVLETLAGQRAVKTHGARTKYLKD
LVEEEAEEAGVALRSTQSTLQAGLAADAWAAPIAMQIYKKHLDPRPGP
CPPELGLGLGQLACCCLHRRAKRRPPMTQVYERLEKLQAVVAGVPGH
SEAASCIPPSPQENSYVSSTGRAHSGAAPWQPLAAPSGASAQAAEQL
QRGPNQPVESDESLGGLSAALRSWHLTPSCPLDPAPLREAGCPQGD
TAGESSWGSGPGSRPTAVEGLALGSSASSSSEPPQIIINPARQKMVQ
KLALYEDGALDSLQLLSSSSLPGLGLEQDRQGPEESDEFQS.

IRAK1, transcript variant 2, is encoded by the following mRNA sequence (NCBI Accession No. NM_{—}001025242 and SEQ ID NO: 30):

cgcggacccggccggcccaggcccgcgcccgccgcggccctgagaggccccggcaggtcccggcccggcg
gcggcagccATGgccggggggccgggcccgggggagcccgcagcccccggcgcccagcacttcttgtacg
aggtgccgccctgggtcatgtgccgcttctacaaagtgatggacgccctggagcccgccgactggtgcca
gttcgccgccctgatcgtgcgcgaccagaccgagctgcggctgtgcgagcgctccgggcagcgcacggcc
agcgtcctgtggccctggatcaaccgcaacgcccgtgtggccgacctcgtgcacatcctcacgcacctgc
agctgctccgtgcgcgggacatcatcacagcctggcaccctcccgccccgcttccgtccccaggcaccac
tgccccgaggcccagcagcatccctgcacccgccgaggccgaggcctggagcccccggaagttgccatcc
tcagcctccaccttcctctccccagcttttccaggctcccagacccattcagggcctgagctcggcctgg
tcccaagccctgcttccctgtggcctccaccgccatctccagccccttcttctaccaagccaggcccaga
gagctcagtgtccctcctgcagggagcccgcccctttccgttttgctggcccctctgtgagatttcccgg
ggcacccacaacttctcggaggagctcaagatcggggagggtggctttgggtgcgtgtaccgggcggtga
tgaggaacacggtgtatgctgtgaagaggctgaaggagaacgctgacctggagtggactgcagtgaagca
gagcttcctgaccgaggtggagcagctgtccaggtttcgtcacccaaacattgtggactttgctggctac
tgtgctcagaacggcttctactgcctggtgtacggcttcctgcccaacggctccctggaggaccgtctcc
actgccagacccaggcctgcccacctctctcctggcctcagcgactggacatccttctgggtacagcccg
ggcaattcagtttctacatcaggacagccccagcctcatccatggagacatcaagagttccaacgtcctt
ctggatgagaggctgacacccaagctgggagactttggcctggcccggttcagccgctttgccgggtcca
gccccagccagagcagcatggtggcccggacacagacagtgcggggcaccctggcctacctgcccgagga
gtacatcaagacgggaaggctggctgtggacacggacaccttcagctttggggtggtagtgctagagacc
ttggctggtcagagggctgtgaagacgcacggtgccaggaccaagtatctgaaagacctggtggaagagg
aggctgaggaggctggagtggctttgagaagcacccagagcacactgcaagcaggtctggctgcagatgc
ctgggctgctcccatcgccatgcagatctacaagaagcacctggaccccaggcccgggccctgcccacct
gagctgggcctgggcctgggccagctggcctgctgctgcctgcaccgccgggccaaaaggaggcctccta
tgacccaggagaactcctacgtgtccagcactggcagagcccacagtggggctgctccatggcagcccct
ggcagcgccatcaggagccagtgcccaggcagcagagcagctgcagagaggccccaaccagcccgtggag
agtgacgagagcctaggcggcctctctgctgccctgcgctcctggcacttgactccaagctgccctctgg
acccagcacccctcagggaggccggctgtcctcagggggacacggcaggagaatcgagctgggggagtgg
cccaggatcccggcccacagccgtggaaggactggcccttggcagctctgcatcatcgtcgtcagagcca
ccgcagattatcatcaaccctgcccgacagaagatggtccagaagctggccctgtacgaggatggggccc
tggacagcctgcagctgctgtcgtccagctccctcccaggcttgggcctggaacaggacaggcaggggcc
cgaagaaagtgatgaatttcagagctgatgtgttcacctgggcagatcccccaaatccggaagtcaaagt
tctcatggtcagaagttctcatggtgcacgagtcctcagcactctgccggcagtgggggtgggggcccat
gcccgcgggggagagaaggaggtggccctgctgttctaggctctgtgggcataggcaggcagagtggaac
cctgcctccatgccagcatctgggggcaaggaaggctggcatcatccagtgaggaggctggcgcatgttg
ggaggctgctggctgcacagacccgtgaggggaggagaggggctgctgtgcaggggtgtggagtagggag
ctggctcccctgagagccatgcagggcgtctgcagcccaggcctctggcagcagctctttgcccatctct
ttggacagtggccaccctgcacaatggggccgacgaggcctagggccctcctacctgcttacaatttgga
aaagtgtggccgggtgcggtggctcacgcctgtaatcccagcactttgggaggccaaggcaggaggatcg
ctggagcccagtaggtcaagaccagccagggcaacatgatgagaccctgtctctgccaaaaaatttttta
aactattagcctggcgtggtagcgcacgcctgtggtcccagctgctggggaggctgaagtaggaggatca
tttatgcttgggaggtcgaggctgcagtgagtcatgattgtatgactgcactccagcctgggtgacagag
caagaccctgtttcaaaaagaaaaaccctgggaaaagtgaagtatggctgtaagtctcatggttcagtcc
tagcaagaagcgagaattctgagatcctccagaaagtcgagcagcacccacctccaacctcgggccagtg
tcttcaggctttactggggacctgcgagctggcctaatgtggtggcctgcaagccaggccatccctgggc
gccacagacgagctccgagccaggtcaggcttcggaggccacaagctcagcctcaggcccaggcactgat
tgtggcagaggggccactacccaaggtctagctaggcccaagacctagttacccagacagtgagaagccc
ctggaaggcagaaaagttgggagcatggcagacagggaagggaaacattttcagggaaaagacatgtatc
acatgtcttcagaagcaagtcaggtttcatgtaaccgagtgtcctcttgcgtgtccaaaagtagcccagg
gctgtagcacaggcttcacagtgattttgtgttcagccgtgagtcacactacatgcccccgtgaagctgg
gcattggtgacgtccaggttgtccttgagtaataaaaacgtatgttgcaataaaaaaaaaaaaaaaaaa.

IRAK1, transcript variant 2, has the following amino acid sequence (NCBI Accession No. NM_{—}001025242 and SEQ ID NO: 31):

MAGGPGPGEPAAPGAQHFLYEVPPWVMCRFYKVMDALEPADWCQFAALI
VRDQTELRLCERSGQRTASVLWPWINRNARVADLVHILTHLQLLRARDII
TAWHPPAPLPSPGTTAPRPSSIPAPAEAEAWSPRKLPSSASTFLSPAFPG
SQTHSGPELGLVPSPASLWPPPPSPAPSSTKPGPESSVSLLQGARPFP
FCWPLCEISRGTHNFSEELKIGEGGFGCVYRAVMRNTVYAVKRLKENAD
LEWTAVKQSFLTEVEQLSRFRHPNIVDFAGYCAQNGFYCLVYGFLPNGSL
EDRLHCQTQACPPLSWPQRLDILLGTARAIQFLHQDSPSLIHGDIKSSNV
LLDERLTPKLGDFGLARFSRFAGSSPSQSSMVARTQTVRGTLAYLPEEY
IKTGRLAVDTDTFSFGVVVLETLAGQRAVKTHGARTKYLKDLVEEEAEEA
GVALRSTQSTLQAGLAADAWAAPIAMQIYKKHLDPRPGPCPPELGLGLG
QLACCCLHRRAKRRPPMTQENSYVSSTGRAHSGAAPWQPLAAPSGASA
QAAEQLQRGPNQPVESDESLGGLSAALRSWHLTPSCPLDPAPLREAGC
PQGDTAGESSWGSGPGSRPTAVEGLALGSSASSSSEPPQIIINPARQKM
VQKLALYEDGALDSLQLLSSSSLPGLGLEQDRQGPEESDEFQS.

IRAK1, transcript variant 3, is encoded by the following mRNA sequence (NCBI Accession No. NM_{—}001025243 and SEQ ID NO: 32):

cgcggacccggccggcccaggcccgcgcccgccgcggccctgagaggccccggcaggtcccggcccggcg
gcggcagccATGgccggggggccgggcccgggggagcccgcagcccccggcgcccagcacttcttgtacg
aggtgccgccctgggtcatgtgccgcttctacaaagtgatggacgccctggagcccgccgactggtgcca
gttcgccgccctgatcgtgcgcgaccagaccgagctgcggctgtgcgagcgctccgggcagcgcacggcc
agcgtcctgtggccctggatcaaccgcaacgcccgtgtggccgacctcgtgcacatcctcacgcacctgc
agctgctccgtgcgcgggacatcatcacagcctggcaccctcccgccccgcttccgtccccaggcaccac
tgccccgaggcccagcagcatccctgcacccgccgaggccgaggcctggagcccccggaagttgccatcc
tcagcctccaccttcctctccccagcttttccaggctcccagacccattcagggcctgagctcggcctgg
tcccaagccctgcttccctgtggcctccaccgccatctccagccccttcttctaccaagccaggcccaga
gagctcagtgtccctcctgcagggagcccgcccctttccgttttgctggcccctctgtgagatttcccgg
ggcacccacaacttctcggaggagctcaagatcggggagggtggctttgggtgcgtgtaccgggcggtga
tgaggaacacggtgtatgctgtgaagaggctgaaggagaacgctgacctggagtggactgcagtgaagca
gagcttcctgaccgaggtggagcagctgtccaggtttcgtcacccaaacattgtggactttgctggctac
tgtgctcagaacggcttctactgcctggtgtacggcttcctgcccaacggctccctggaggaccgtctcc
actgccagacccaggcctgcccacctctctcctggcctcagcgactggacatccttctgggtacagcccg
ggcaattcagtttctacatcaggacagccccagcctcatccatggagacatcaagagttccaacgtcctt
ctggatgagaggctgacacccaagctgggagactttggcctggcccggttcagccgctttgccgggtcca
gccccagccagagcagcatggtggcccggacacagacagtgcggggcaccctggcctacctgcccgagga
gtacatcaagacgggaaggctggctgtggacacggacaccttcagctttggggtggtagtgctagagacc
ttggctggtcagagggctgtgaagacgcacggtgccaggaccaagtatctggtgtacgagaggctagaga
agctgcaggcagtggtggcgggggtgcccgggcattcggaggccgccagctgcatccccccttccccgca
ggagaactcctacgtgtccagcactggcagagcccacagtggggctgctccatggcagcccctggcagcg
ccatcaggagccagtgcccaggcagcagagcagctgcagagaggccccaaccagcccgtggagagtgacg
agagcctaggcggcctctctgctgccctgcgctcctggcacttgactccaagctgccctctggacccagc
acccctcagggaggccggctgtcctcagggggacacggcaggagaatcgagctgggggagtggcccagga
tcccggcccacagccgtggaaggactggcccttggcagctctgcatcatcgtcgtcagagccaccgcaga
ttatcatcaaccctgcccgacagaagatggtccagaagctggccctgtacgaggatggggccctggacag
cctgcagctgctgtcgtccagctccctcccaggcttgggcctggaacaggacaggcaggggcccgaagaa
agtgatgaatttcagagctgatgtgttcacctgggcagatcccccaaatccggaagtcaaagttctcatg
gtcagaagttctcatggtgcacgagtcctcagcactctgccggcagtgggggtgggggcccatgcccgcg
ggggagagaaggaggtggccctgctgttctaggctctgtgggcataggcaggcagagtggaaccctgcct
ccatgccagcatctgggggcaaggaaggctggcatcatccagtgaggaggctggcgcatgttgggaggct
gctggctgcacagacccgtgaggggaggagaggggctgctgtgcaggggtgtggagtagggagctggctc
ccctgagagccatgcagggcgtctgcagcccaggcctctggcagcagctctttgcccatctctttggaca
gtggccaccctgcacaatggggccgacgaggcctagggccctcctacctgcttacaatttggaaaagtgt
ggccgggtgcggtggctcacgcctgtaatcccagcactttgggaggccaaggcaggaggatcgctggagc
ccagtaggtcaagaccagccagggcaacatgatgagaccctgtctctgccaaaaaattttttaaactatt
agcctggcgtggtagcgcacgcctgtggtcccagctgctggggaggctgaagtaggaggatcatttatgc
ttgggaggtcgaggctgcagtgagtcatgattgtatgactgcactccagcctgggtgacagagcaagacc
ctgtttcaaaaagaaaaaccctgggaaaagtgaagtatggctgtaagtctcatggttcagtcctagcaag
aagcgagaattctgagatcctccagaaagtcgagcagcacccacctccaacctcgggccagtgtcttcag
gctttactggggacctgcgagctggcctaatgtggtggcctgcaagccaggccatccctgggcgccacag
acgagctccgagccaggtcaggcttcggaggccacaagctcagcctcaggcccaggcactgattgtggca
gaggggccactacccaaggtctagctaggcccaagacctagttacccagacagtgagaagcccctggaag
gcagaaaagttgggagcatggcagacagggaagggaaacattttcagggaaaagacatgtatcacatgtc
ttcagaagcaagtcaggtttcatgtaaccgagtgtcctcttgcgtgtccaaaagtagcccagggctgtag
cacaggcttcacagtgattttgtgttcagccgtgagtcacactacatgcccccgtgaagctgggcattgg
tgacgtccaggttgtccttgagtaataaaaacgtatgttgcaataaaaaaaaaaaaaaaaaa.

IRAK1, transcript variant 3, has the following amino acid sequence (NCBI Accession No. NM_{—}001025243 and SEQ ID NO: 33):

MAGGPGPGEPAAPGAQHFLYEVPPWVMCRFYKVMDALEPADWCQFAAL
IVRDQTELRLCERSGQRTASVLWPWINRNARVADLVHILTHLQLLRARDI
ITAWHPPAPLPSPGTTAPRPSSIPAPAEAEAWSPRKLPSSASTFLSPAFP
GSQTHSGPELGLVPSPASLWPPPPSPAPSSTKPGPESSVSLLQGARPFP
FCWPLCEISRGTHNFSEELKIGEGGFGCVYRAVMRNTVYAVKRLKENA
DLEWTAVKQSFLTEVEQLSRFRHPNIVDFAGYCAQNGFYCLVYGFLPN
GSLEDRLHCQTQACPPLSWPQRLDILLGTARAIQFLHQDSPSLIHGDIK
SSNVLLDERLTPKLGDFGLARFSRFAGSSPSQSSMVARTQTVRGTLA
YLPEEYIKTGRLAVDTDTFSFGVVVLETLAGQRAVKTHGARTKYLVYER
LEKLQAVVAGVPGHSEAASCIPPSPQENSYVSSTGRAHSGAAPWQPLA
APSGASAQAAEQLQRGPNQPVESDESLGGLSAALRSWHLTPSCPLDP
APLREAGCPQGDTAGESSWGSGPGSRPTAVEGLALGSSASSSSEP
PQIIINPARQKMVQKLALYEDGALDSLQLLSSSSLPGLGLEQDRQGPE
ESDEFQS.

Silencing Expression with MicroRNAs

The present invention comprises compositions with means to inhibit the activity of IL-1α, IL-1β, IL-1R1, IL-1R2, IL-1Ra3, IL-1RAP, or IRAK1, by delivering microRNA (miRNA) molecules to an ocular or adnexal tissue with an appropriate pharmaceutical carrier. Compositions that comprise a miRNA targeted to either IL-1α, IL-1β, IL-1R1, IL-1R2, IL-1Ra3, IL-1RAP, or IRAK1 antagonize the function of IL-1R1. The composition comprises one or more miRNA(s) that bind to one or more regions of IL-1α, IL-1β, IL-1R1, IL-1R2, IL-1Ra3, IL-1RAP, or IRAK1. The following table contains exemplary miRNAs that have been shown to partially or completely silence the expression of human IL-1α or IL-1R1.

TABLE 1
Summary of miRNAs, their human target genes,
nucleotide sequences, and their
sequence identifier numbers.

| Target | | Polynucleotide sequence | SEQ |
|---|---|---|---|
| Gene | miRNA | (5′ to 3′) | ID NO: |
| IL-1α | miR-30c | UGUAAACAUCCUACACUCUCAGC | 34 |
| IL-1α | miR-30b | UGUAAACAUCCUACACUCAGC | 35 |
| IL-1α | miR-30a-5p | UGUAAACAUCCUCGACUGGAAGC | 36 |
| IL-1α | miR-24 | UGGCUCAGUUCAGCAGGAACAG | 37 |
| IL-1R1 | miR-135b | UAUGGCUUUUCAUUCCUAUGUG | 38 |
| IL-1R1 | miR-326 | CCUCUGGGCCCUUCCUCCAG | 39 |
| IL-1R1 | miR-184 | UGGACGGAGAACUGAUAAGGGU | 40 |
| IL-1R1 | miR-214 | ACAGCAGGCACAGACAGGCAG | 41 |
| IL-1R1 | miR-203 | GUGAAAUGUUUAGGACCACUAG | 42 |
| IL-1R1 | miR-331 | GCCCCUGGGCCUAUCCUAGAA | 43 |
| IL-1R1 | miR-205 | UCCUUCAUUCCACCGGAGUCUG | 44 |

### IL-1 and IL-1R-Mediated Signaling

As used herein, the phrase “inhibit an activity of an inflammatory interleukin-1 cytokine” is meant to describe the inhibition, prevention, diminution, reduction, decrease, repression, or interruption intracellular signaling initiated, communicated, or transduced from an IL-1 receptor. In one aspect of the invention, inhibition, prevention, diminution, reduction, decreases, repression, or interruption of intracellular signaling initiated, communicated, or transduced from an IL-1 receptor is achieved by preventing or decreasing binding of an IL-1 cytokine to an IL-1R, e.g., preventing or decreasing binding of human IL-1β or IL-1α to human IL-1R1. Alternatively, or in addition, transduction of intracellular signaling from an IL-1R is prevented by removing, silencing, or mutating a downstream effector or target within a signaling cascade. The expression and/or function or activity of downstream effectors and/or targets are removed (e.g., deleted, knocked-out, sequestered, denatured, degraded, etc.), silenced (degraded, transcriptionally or translationally repressed), or mutated (nucleotide or amino acid sequence encoding the active product is altered to encode a non-functional product) by genetic modification or administration of a therapeutic compound.

Exemplary downstream effectors and/or targets include, but are not limited to, one or more isoforms or homologs of an IL-1 (interleukin 1), an IL-1α (interleukin 1 alpha), an IL-1β (interleukin 1 beta), an IL-1R (interleukin 1 receptor, type I), an IL-1Ra (IIL-1R antagonist), an IL-1RAcP (IL-1R accessory protein), a TOLLIP (TOLL interacting protein), an IRAK1 (IL-1R associated kinase 1), an IRAK2 (IL-1R associated kinase 2), an IRAK 3 (IL-1R associated kinase 3), a MYD88 (myeloid differentiation primary response gene 88), an ECSIT (evolutionarily conserved signaling intermediate in Toll pathways), a TRAF6 (TNF-receptor associated factor 6), a MEKK1 (MAP ERK kinase kinase 1), a TAB1 (TAK1 binding protein 1), a TAK1 (transforming growth factor b activated kinase 1), a NIK (NFkB Inducing Kinase), a RKIP (Raf kinase inhibitor protein), a MEK3 (Mitogen-Activated Protein Kinase Kinase 3; MEK3 or MKK3), a MEK6 (Mitogen-Activated Protein Kinase Kinase 6; MEK6 or MKK6), a MAPK14 (mitogen activated protein kinase 14), a MAPK8 (mitogen activated protein kinase 8), a MEKK1 (mitogen activated protein kinase kinase kinase 1), a MAP3K14 (mitogen activated protein kinase kinase kinase 14), a MEKK7 (mitogen activated protein kinase kinase kinase 7 or MKK7), a MAP3K71P1 (mitogen activated protein kinase kinase kinase 7 interacting protein 1), a JNK (Jun N-terminal kinase), p38 (also known as p38 MAPK or p38 mitogen activated protein kinase), cJUN (jun oncogene), AP-1 (activator protein 1; transcription factor), IL-6 (interleukin 6, also known as interferon beta 2), TNFα (tumor necrosis factor-alpha), a TNF (tumor necrosis factor superfamily member), an IFNα (interferon alpha, interferon alpha 1), an IFNβ (interferon beta, interferon beta 1), a TGFβ1 (transforming growth factor beta 1), a TGFβ2 (transforming growth factor beta 2), a TGFβ3 (transforming growth factor beta 3), an IKKα (inhibitor of kappa light polypeptide gene enhancer in B-cells, kinase alpha), an IKKβ (inhibitor of kappa light polypeptide gene enhancer in B-cells, kinase beta), a IκBα (nuclear factor of kappa light polypeptide gene enhancer in B-cells inhibitor, alpha), a Chuk (conserved helix-loop-helix ubiquitous kinase), and a NFκB (nuclear factor of kappa light polypeptide gene enhancer in B-cells 1; also known as p105). Additional signaling molecules and relationships are defined by O'Neill, L. A. J. and Greene, C. 1998. Journal of Leukocyte Biology. 63: 650-657.

The inhibition of an activity of an interleukin-1 cytokine is determined by sampling the cornea and determining the abundance of a polynucleotide or polypeptide which encodes for component of an IL-1R signaling cascade. An increase or decrease in the abundance of a polynucleotide or polypeptide which encodes for component of an IL-1R signaling cascade following administration of a therapeutic composition of the invention compared to the abundance of the component of an IL-1R signaling cascade prior to the administration indicates inhibition of an activity of an inflammatory interleukin-1 cytokine.

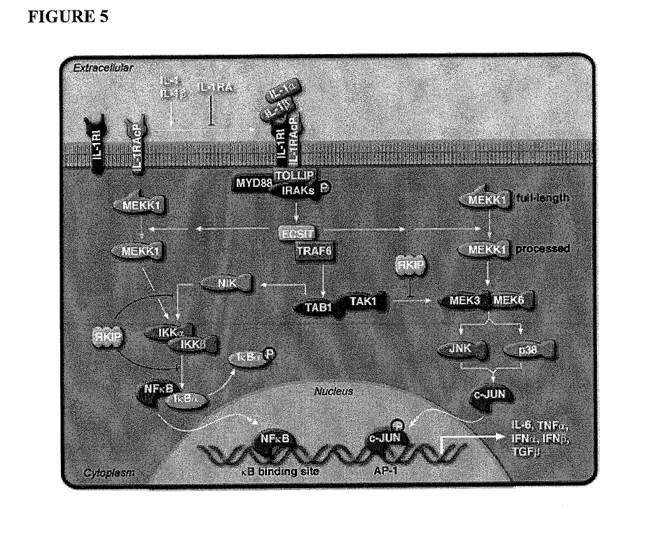

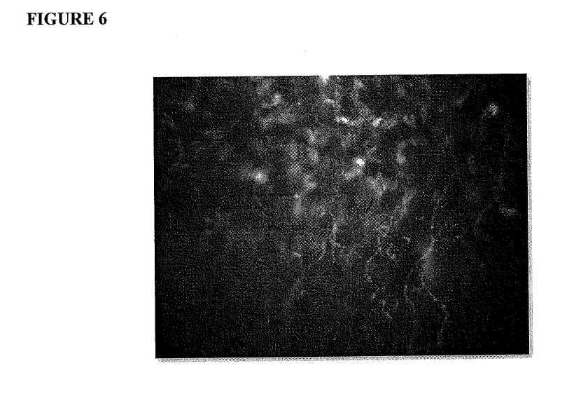

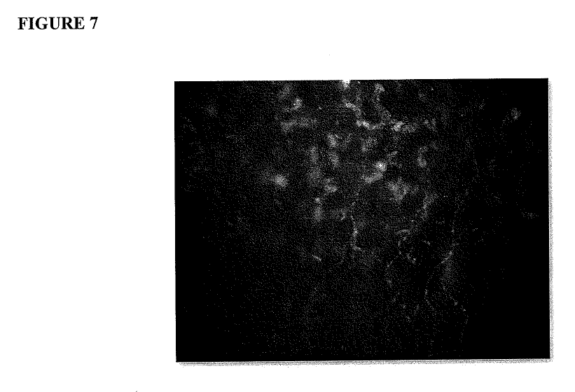

Specifically, FIG. 5 shows the functional interrelationships between components of two exemplary signaling cascades. The arrows between components in this figure indicate that the component preceding the arrow activates the component following the arrow. Conversely, the blunted lines indicated that the component preceding the blunted line inhibits the activity or function of the component following the blunted line.

Briefly, the IL-1R, type I, binds IL-1β, however, IL-1R requires the IL-1 receptor accessory protein (IL-1 RAcP) to transduce a signal. IL-1 binding causes activation of two kinases, IRAK-1 and IRAK-2, associated with the IL-1 receptor complex. IRAK-1 (IL-1 Receptor Associated Kinase) activates and recruits TRAF6 to the IL-1 receptor complex. TRAF6 activates two pathways, one leading to NF-kB activation and another leading to c-jun activation. The TRAF associated protein ECSIT leads to c-Jun activation through the Map kinase/JNK signaling system. TRAF6 also signals through the TAB1/TAK1 kinases to trigger the degradation of 1-kB, and activation of NF-kB.

For instance, in certain embodiments of the invention, a decrease in the abundance or absence of the processed form of MEKK1, a decrease in the abundance or absence of phosphorylated IκBα, a decrease in the abundance or absence of phosphorylated c-JUN, a decrease in the abundance or absence of ICAM-1, or a decrease in the abundance or absence of IL-6, TNFα, IFNα, IFNβ, TGFβ is indicative of inhibition of an interleukin-1 cytokine. Similarly, a decrease or absence of activity or function of any of the above-listed components is indicative of inhibition of an interleukin-1 cytokine.

### Pharmaceutically-Appropriate Carriers

Exemplary compounds incorporated to facilitate and expedite local delivery of topical compositions into ocular or adnexal tissues include, but are not limited to, alcohol (ethanol, propanol, and nonanol), fatty alcohol (lauryl alcohol), fatty acid (valeric acid, caproic acid and capric acid), fatty acid ester (isopropyl myristate and isopropyl n-hexanoate), alkyl ester (ethyl acetate and butyl acetate), polyol (propylene glycol, propanedione and hexanetriol), sulfoxide (dimethylsulfoxide and decylmethylsulfoxide), amide (urea, dimethylacetamide and pyrrolidone derivatives), surfactant (sodium lauryl sulfate, cetyltrimethylammonium bromide, polaxamers, spans, tweens, bile salts and lecithin), terpene (d-limonene, alpha-terpeneol, 1,8-cineole and menthone), and alkanone (N-heptane and N-nonane). Moreover, topically-administered compositions comprise surface adhesion molecule modulating agents including, but not limited to, a cadherin antagonist, a selectin antagonist, and an integrin antagonist.

Optionally, the composition further contains a compound selected from the group consisting of a physiological acceptable salt, poloxamer analogs with carbopol, carbopol/hydroxypropyl methyl cellulose (HPMC), carbopol-methyl cellulose, N-acetyl cysteine, carboxymethylcellulose (CMC), hyaluronic acid, cyclodextrin, and petroleum.

### EXAMPLES

### Example 1

### Effect of IL-1 Blockade on Central Nerve Regeneration in Epithelial Disease

It was observed that subjects with the most severe clinical signs of dry eye reported feeling surprising improvement after administration of IL-1 blockade. Upon further investigation, patients with severe dry eye were found to also suffer after administration of IL-1 blockade corneal nerve damage or even loss of nerve function. To better elucidate the mechanism of this unexpected improvement following treatment, the contribution of nerve damage and function to the normal and pathological processes of tear production and dry eye was investigated.

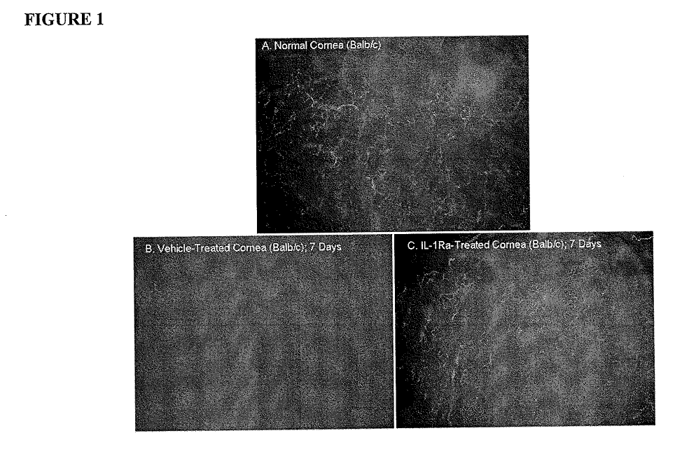

To examine the role of IL-1 blockade in corneal nerve homeostasis, regeneration of the corneal subbasal nerve plexus and terminal epithelial branches after epithelial debridement was measured. Mice were anesthetized, and a 2-mm circular corneal epithelial defect was made in one eye of each anesthetized mouse using a scalpel blade. One group of mice (n=5) was treated with topical eye drop of IL-1Ra 2.5% mixed with carboxymethylcellulose 1%, 3 times a day for 7 days. The control group (n=5) was treated using vehicle (carboxymethylcellulose 1%), 3 times a day for 7 days. After 7 days, animals were euthanized, and corneas were removed and fixed in 4% Para formaldehyde for 40 minutes at room temperature. Corneas were washed with PBS. Permeabilization and blocking was achieved with 2-hour incubation in 2% BSA in PBS and 0.2% Triton X-100. Corneas were incubated with rabbit anti-mouse B III tubulin primary antibody at a dilution of 1:200 for 16 hours at 4°. After three washes in PBS corneas were incubated in goat anti-rabbit secondary antibody conjugated to rhodamine at a dilution of 1:200 for 2 hours at room temperature. The corneas were coverslipped with mounting medium and imaged. The corneas were photographed at the level of the subbasal nerve plexus and terminal branching in cornea paracentral regions in the area of the wound (FIG. 1). For the treated eye, nerve density at the level of terminal branching was normalized to the contralateral normal cornea. The results of this experiment showed that in the IL-1Ra-treated group, the percent density of regenerated nerve at the level of basal epithelial cell was 80% compared to 15% in the vehicle-treated group.

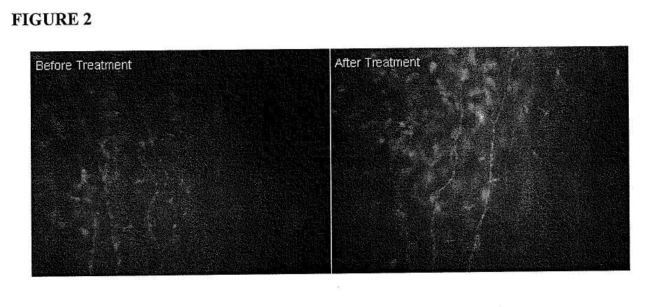

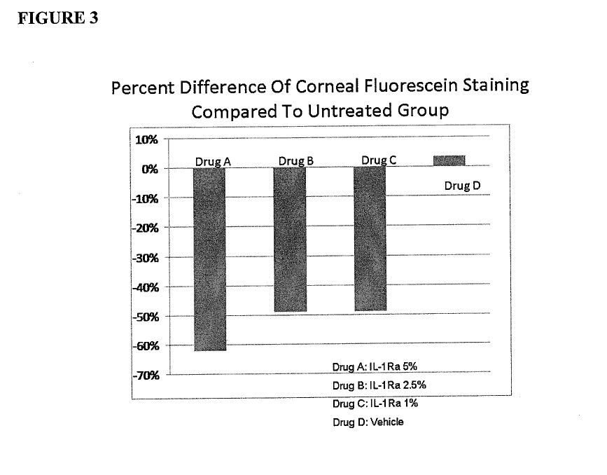

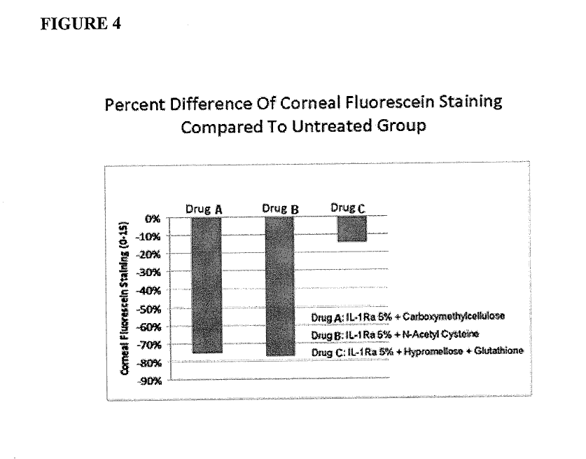

In a clinical human study in patients with dry eye-associated ocular surface disease using in-vivo confocal microscopy (Confoscan 4; Nidek Technologies), corneal nerve morphology at the level of subbasal corneal nerve was assessed before and after one-month treatment with topical IL-1Ra 2.5% three times a day (FIG. 2). After the treatment with IL-1 blockade in a total of 6 patients, the sum of the length of the nerves per image (nerve fiber density) was increased by 25% compared to baseline. The improvement in corneal nerve density was correlated with the reduction of signs and symptoms of dry eye in these patients.

IL-1 blockade, therefore, helps the epithelium improve its maintenance of corneal health by increasing nerve regeneration, which in turn breaks the vicious cycle of epithelial disease and nerve damage. Thus, IL-1 blockade can be used in all ophthalmologic conditions that affect the corneal epithelial health, including all forms of dry eye syndrome, all form of ocular surface diseases, corneal surgeries such as refractive surgeries (PRK, LASEK, Epi-LASIK, LASIK), corneal transplantation, epithelial debridement, ocular surface reconstruction, herpetic keratitis, neurotrophic keratitis, exposure keratopathy, diabetes mellitus, trigeminal nerve damage. Non-ophthalmic applications of IL-1 blockade would be all forms of peripheral neuropathy including diabetic peripheral neuropathy.

### Example 2

### Effect of IL-1 Blockade on Dry-Eye Induced Lymphangiogenesis

Corneal angiogenesis is involved in the pathogenesis of adaptive immunity to corneal antigens and in inducing ocular surface disease. Lymphatic vessels are crucial for migration of resident antigen presenting cells to the draining lymph nodes and induction of adaptive immunity, but there is no information about the role of lymphangiogenesis in dry-eye associated ocular surface disease. In a mouse model of dry eye, it was determined whether IL-1 blockade can reduce dry-eye induced lymphangiogenesis. Dry eye was induced in female C57BL/6 mice (n=10) by exposure to the controlled environment chamber and to systemic scopolamine. After 2 days, one group of mice (n=5) received topical IL-1Ra 2.5% mixed with carboxymethylcellulose 1% three times a day, and the second group received only the vehicle (carboxymethylcellulose 1%). After 5 days of treatment, animals were euthanized, and corneas were removed and fixed in 4% Para formaldehyde for 40 minutes at room temperature. Immunohistochemical staining for lymphatic vessels and blood vessels was performed on corneal flat mounts. Immunohistochemical staining against LYVE-1 (lymphatic endothelium-specific hyaluronic acid receptor) was performed with purified antibody followed by rhodamine conjugated secondary antibody. Immunohistochemical staining was also performed with FITC-conjugated CD31. To quantify the level of blood vessel formation and lymphatic vessel formation, low magnification (2×) micrographs were captured and the area covered by lymph neovessels were calculated and expressed as % of total corneal area. IL-1Ra-treated corneas showed on average 60% less lymphangiogenesis activity compared to vehicle-treated corneas. In addition, blockade of IL-1 led to the immune cells having a lower level of maturity, which renders them less able to sensitize T cells.

The results of this experiment show that the compounds having the inhibitory activity against IL-1 on prevention of dry-eye associated lymphangiogenesis are useful to minimize the induction of adaptive immunity and autoimmunity in dry-eye associated ocular surface disease.

### Example 3

### Concentration of Topical IL-1Ra

With respect to effects of IL-1 blockade on dry eye disease, different concentrations (1%, 2.5%, and 5%) of topical IL-1Ra mixed with carboxymethylcellulose 1% were tested in a mouse model of dry eye. Dry eye was induced in female C57BL/6 mice (n=20) by exposure to the controlled environment chamber and to systemic scopolamine. After 2 days, mice were divided in 5 groups. The first, second, and third groups of mice (4 mice in each group) received topical IL-1Ra 5%, 2.5%, and 1% respectively, mixed with carboxymethylcellulose 1% in frequency of 3 times a day; the fourth group (n=4) received vehicle (carboxymethylcellulose 1%) in frequency of 3 times a day; and the fifth group (n=4) left untreated. After 5 days of treatment, corneal fluorescein staining was performed by applying 0.5 μL of 1% fluorescein by micropipette into the inferior conjunctival sac of the mouse eye. The cornea was examined with a slit lamp biomicroscope in cobalt blue light 3 minutes after fluorescein instillation. Punctuate staining was recorded in a masked fashion with a standardized (National Eye Institute) grading system of 0 to 3 for each of the five areas in which the corneal surface was divided. This experiment showed that all concentrations of IL-1Ra (1%, 2.5%, and 5%) can decrease the corneal fluorescein staining score, however, percent reduction of corneal fluorescein staining was modestly higher in the group that received topical IL-1Ra with concentration of 5%.

### Example 4

### Formulation of Topical IL-1Ra

With respect to effects of IL-1 blockade on dry eye disease, IL-1Ra 5% mixed with different vehicles was tested in a mouse model of dry eye. Dry eye was induced in female C57BL/6 mice (n=20) by exposure to the controlled environment chamber and to systemic scopolamine. After 2 days, mice were divided in 4 groups. The first group of mice (n=5) received topical IL-1Ra 5% mixed with carboxymethylcellulose 1% in frequency of 3 times a day; the second group (n=5) received topical IL-1Ra 5% mixed with N-acetyl cysteine 10% in frequency of 3 times a day; the third group (n=5) received topical IL-1Ra 5% mixed with hypromellose (hydroxypropyl methylcellulose) 0.3% and glutathione 0.3 mmol/L; and the fourth group (n=5) left untreated. After 5 days of treatment, corneal fluorescein staining was performed by applying 0.5 μL of 1% fluorescein by micropipette into the inferior conjunctival sac of the mouse eye. The cornea was examined with a slit lamp biomicroscope in cobalt blue light 3 minutes after fluorescein instillation. Punctuate staining was recorded in a masked fashion with a standardized (National Eye Institute) grading system of 0 to 3 for each of the five areas in which the corneal surface was divided. This experiment showed that percent reduction of corneal fluorescein staining was significantly higher in the group received topical IL-1Ra 5% mixed with N-acetyl cysteine 10% and the group received topical IL-1Ra 5% mixed with carboxymethylcellulose 1%.

### Example 5

### Minimization or Prevention of Corneal Nerve Degeneration

To examine the role of IL-1 blockade in corneal nerve homeostasis, minimization and prevention of nerve degeneration of the corneal subbasal nerve plexus and terminal epithelial branches was measured. One group of mice (n=5) was treated with topical eye drop of IL-1Ra, 2.5% mixed with carboxymethylcellulose 1%, 3 times a day for 7 days. The control group (n=5) was treated using vehicle (carboxymethylcellulose 1%), 3 times a day for 7 days. Mice were then anesthetized, and a 2-mm circular corneal epithelial defect was made in one eye of each anesthetized mouse using a scalpel blade. After 7 days, animals were euthanized, and corneas were removed and fixed in 4% Para formaldehyde for 40 minutes at room temperature. Corneas were washed with PBS. Permeabilization and blocking was achieved with 2-hour incubation in 2% BSA in PBS and 0.2% Triton X-100. Corneas were incubated with rabbit anti-mouse β III tubulin primary antibody at a dilution of 1:200 for 16 hours at 4°. After three washes in PBS corneas were incubated in goat anti-rabbit secondary antibody conjugated to rhodamine at a dilution of 1:200 for 2 hours at room temperature. The corneas were coverslipped with mounting medium and imaged. The corneas are photographed at the level of the subbasal nerve plexus and terminal branching in cornea paracentral regions in the area of the wound. For the treated eye, nerve density at the level of terminal branching is normalized to the contralateral normal cornea. The results of this experiment show that that in the IL-1Ra-treated group, the percent density of degenerated nerve at the level of basal epithelial cell was decreased compared to the vehicle-treated group.

IL-1 blockade, therefore, can help the epithelium improve its maintenance of corneal health by minimizing or preventing nerve degeneration which in turn can break the vicious cycle of epithelial disease and nerve damage. Thus, IL-1 blockade can be used in all ophthalmologic conditions that affect the corneal epithelial health including all forms of dry eye syndrome, all form of ocular surface diseases, corneal surgeries such as refractive surgeries (PRK, LASEK, Epi-LASIK, LASIK), corneal transplantation, epithelial debridement, ocular surface reconstruction, herpetic keratitis, neurotrophic keratitis, exposure keratopathy, diabetes mellitus, trigeminal nerve damage. Non-ophthalmic applications of IL-1 blockade would be all forms of peripheral neuropathy including diabetic peripheral neuropathy.

### Example 6

### IL-1 and IL-17 Blockade Provides Unexpected Protection of Corneal Nerves

As described in WO/2009/089036, which is incorporated herein by reference, the method comprises administration of a compound that inhibits binding of an inflammatory IL-17 cytokine to the IL-17 receptor complex.

A method for regenerating corneal nerves is also carried out by locally administering to an eye of a subject a composition comprising a polynucleotide, a polypeptide, an antibody, a compound, or a small molecule that inhibits or modifies the transcription, transcript stability, translation, modification, localization, secretion, or function of a polynucleotide or polypeptide encoding an inflammatory interleukin-17 cytokine or any component of the IL-17 receptor complex.

The composition may comprise a neutralizing or function-blocking antibody against IL-17 and/or a receptor complex. The neutralizing or function-blocking antibody against IL-17 may be a reformulated or humanized derivative of or bind to the epitope of human IL-17 affinity purified polyclonal antibody (Catalog #AF-317-NA, R&D Systems), human IL-17 allophycocyanin monoclonal antibody (clone 41802) (Catalog #IC3171A, R&D Systems), human IL-17 biotinylated affinity purified polyclonal antibody (Catalog #BAF317, R&D Systems), human IL-17 monoclonal antibody (clone 41802) (Catalog #MAB3171, R&D Systems), human IL-17 monoclonal antibody (clone 41809)(Catalog #MAB317, R&D Systems), human IL-17 phycoerythrin monoclonal antibody (clone 41802) (Catalog #IC3171P, R&D Systems), mouse IL-17 affinity purified polyclonal antibody (Catalog #AF-421-NA, R&D Systems), mouse IL-17 biotinylated affinity purified polyclonal antibody (Catalog #BAF421, R&D Systems), mouse IL-17 monoclonal antibody (clone 50101) (Catalog #MAB721, R&D Systems), or mouse IL-17 monoclonal antibody (clone 50104) (Catalog #MAB421, R&D Systems). Preferably, the neutralizing or function-blocking antibody against IL-17 may be a reformulated or humanized derivative of or bind to the epitope of monoclonal anti-human IL-17 antibody, (Clone: 41809, Catalog #MAB317, R&D Systems), anti-human IL-17 antibody, polyclonal raised in Goat, (Catalog #AF-317-NA, R&D Systems), or recombinant human IL-17 RTFc chimera (Catalog #177-IR, R&D Systems).

By “reformulate” is meant altering the composition to make it suitable for topical administration, subconjunctival administration, episcleral space administration, subcutaneous administration, or intraductal administration. Preferred formulations are in the form of a solid, a paste, an ointment, a gel, a liquid, an aerosol, a mist, a polymer, a contact lens, a film, an emulsion, or a suspension. In one aspect, the formulations are administered topically, e.g., the composition is delivered and directly contacts the eye. Optionally, the compositions are administered with a pharmaceutically acceptable liquid carrier, e.g., a liquid carrier, which is aqueous or partly aqueous. Alternatively, the compositions are associated with a liposome (e.g., a cationic or anionic liposome).

The neutralizing or function-blocking antibody against an IL-17 receptor (I1-17R) may be a reformulated or humanized derivative of or bind to the epitope of human IL-17R affinity purified polyclonal antibody (Catalog #AF 177, R&D Systems), human IL-17R allophycocyanin monoclonal antibody (clone 133617) (Catalog #FAB177A, R&D Systems), human IL-17R biotinylated affinity purified polyclonal antibody (Catalog #BAF 177, R&D Systems), human IL-17R fluorescein monoclonal antibody (clone 133617) (Catalog #FAB177F, R&D Systems), human IL-17R monoclonal antibody (clone 133617) (Catalog #MAB 177, R&D Systems), human IL-17R monoclonal antibody (clone 133621) (Catalog #MAB 1771, R&D Systems), human IL-17R phycoerythrin monoclonal antibody (clone 133617) (Catalog #FAB 177P, R&D Systems), mouse IL-17R affinity purified polyclonal antibody (Catalog #AF448A, R&D Systems), mouse IL-17R biotinylated affinity purified polyclonal antibody (Catalog #BAF448, R&D Systems), or mouse IL-17R monoclonal antibody (clone 105828) (Catalog #MAB448, R&D Systems).

The neutralizing or function-blocking antibody against an IL-17 may be a reformulated or humanized derivative of, or bind to the epitope of, one or more mouse anti-IL-17A (SKU #s including but not limited to, 7172, 7173, 7175, 7177, 8171, 7371, 7971, and 7370, eBioscience) or mouse anti-IL-17F (SKU #s including, but not limited to, 7471 and 8471, eBioscience). The neutralizing or function-blocking antibody against an IL-17 may be a reformulated or humanized derivative of one or more human anti-IL-17A (SKU #s including, but not limited to, 7178, 7179, 8179, 7176, 7976, and 7876 or human anti-IL-17F SKU #s including, but not limited to, 8479, eBioscience). Preferably, the neutralizing or function-blocking antibody against an IL-17 may be a reformulated or humanized derivative of, or bind to the epitope of functional grade purified anti-human IL-17A antibody (Clone: eBio64CAP17, Catalog #16-7178. eBioscience).

Alternatively, the composition may comprise an intrabody that binds to the IL-17 receptor complex or any synthetic intermediate of IL-17 or the IL-17 receptor complex. The composition may alternatively, or in addition, comprise a soluble fragment of the IL-17 receptor complex which binds IL-17.

Exemplary polypeptides include, but are not limited to, fusion and/or chimeric proteins capable of disrupting IL-17 function. Moreover, the composition comprises morpholino antisense oligonucleotides, microRNAs (miRNAs), short hairpin RNA (shRNA), or short interfering RNA (siRNA) to silence gene expression.

Contemplated function-blocking antibodies targeted against an IL-17 cytokine or an IL-17 receptor are monoclonal or polyclonal. The contemplated antibody binds to one or more sequences within an IL-17 or IL-17 receptor polypeptide. The antibody is alternatively an intrabody. In some embodiments, the antibody comprises a single chain, a humanized, a recombinant, or a chimeric antibody. One or more compounds are directly or indirectly conjugated onto this antibody.

Antagonists of IL-17 and/or its receptor complex are administered either simultaneously or sequentially with an antagonist of IL-1 and/or its receptor.

Human IL-17 is encoded by the mRNA sequence of NCBI Accession No. NM_{—}002190, alternatively called IL-17A. Human IL-17 is encoded by the amino acid sequence of NCBI Accession No. NM_{—}002190, alternatively called IL-17A. Human IL-17B is encoded by the mRNA sequence of NCBI Accession No. NM_{—}014443. Human IL-17B is encoded by the amino acid sequence of NCBI Accession No. NM_{—}014443. Human IL-17C is encoded by the mRNA sequence of NCBI Accession No. NM_{—}013278. Human IL-17C is encoded by the amino acid sequence of NCBI Accession No. NM_{—}013278. Human IL-17D is encoded by the mRNA sequence of NCBI Accession No. NM_{—}138284. Human IL-17D is encoded by the amino acid sequence of NCBI Accession No. NM_{—}138284. Human IL-17E is encoded by the mRNA sequence of NCBI Accession No. AF305200. Human IL-17E is encoded by the amino acid sequence of NCBI Accession No. AF305200. Human IL-17F is encoded by the mRNA sequence of NCBI Accession No. NM 052872. Human IL-17F is encoded by the amino acid sequence of NCBI Accession No. NM_{—}052872.

IL-17 receptor antagonist (IL-17RA) is encoded by the mRNA sequence of NCBI Accession No. NM 014339. IL-17RA is encoded by the amino acid sequence of NCBI Accession No. NMJ4339. IL-17RB is encoded by the mRNA sequence of NCBI Accession No. NM_{—}018725. IL-17RB is encoded by the amino acid sequence of NCBI Accession No. NM_{—}018725. IL-17RC, transcript variant 1, is encoded by the mRNA sequence of NCBI Accession No. NM_{—}1 53461. IL-17RC, transcript variant 1, is encoded by the amino acid sequence of NCBI Accession No. NM_{—}153461. IL-17RC, transcript variant 2, is encoded by the mRNA sequence of NCBI Accession No. NMJ 53460. IL-17RC, transcript variant 2, is encoded by the amino acid sequence of NCBI Accession No. NM_{—}153460. IL-17RC, transcript variant 3, is encoded by the mRNA sequence of NCBI Accession No. NM_{—}032732. IL-17RC, transcript variant 3, is encoded by the amino acid sequence of NCBI Accession No. NM_{—}032732. IL-17RD, transcript 1, is encoded by the mRNA sequence of NCBI Accession No. NMJ)01080973. IL-17RD, transcript 1, is encoded by the amino acid sequence of NCBI Accession No. NMJ)O1080973. IL-17RD, transcript 2, is encoded by the mRNA sequence of NCBI Accession No. NM OI 7563. IL-17RD, transcript 2, is encoded by the amino acid sequence of NCBI Accession No. NM_{—}017563. IL-17RE, transcript variant 1, is encoded by the mRNA sequence of NCBI Accession No. NM 1 53480. IL-17RE, transcript variant 1, is encoded by the amino acid sequence of NCBI Accession No. NM_{—}153480. IL-17RE, transcript variant 2, is encoded by the mRNA sequence of NCBI Accession No. NM_{—}153481. IL-17RE, transcript variant 2, is encoded by the amino acid sequence of NCBI Accession No. NM_{—}153481. IL-17RE, transcript variant 5, is encoded by the mRNA sequence of NCBI Accession No. NM_{—}153483. IL-17RE, transcript variant 5, is encoded by the amino acid sequence of NCBI Accession No. NM_{—}153483.

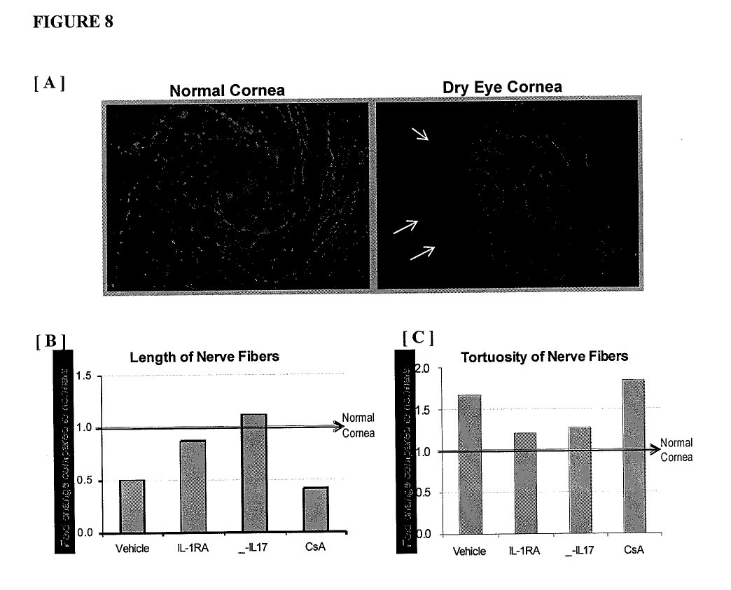

To study the effect of IL-17 blockade on corneal neuropathy, an art-recognized murine model of dry eye disease (DED) was used. As described above, DED was induced in female C57BL/6 mice by exposure to a desiccating environment in a controlled environment chamber and to systemic scopolamine (FIG. 8A). After induction of DED for 9-10 days, whole mount corneas were immunostained for nerve-specific β-tubulin-III, and then epifluorescence micrographs were captured and processed under an automated Matlab based software nerve quantification. Nerve fiber length and nerve fiber tortuosity (indices of neuroregeneration/neurodegeneration) were analyzed as described below.

Both the anti-cytokine therapies, including IL-1Ra and anti-IL17 prevented loss of nerve fibers in DED corneas (FIG. 8B). By contrast, use of a lubricating drop (“Vehicle”) or CsA showed ˜2-fold decrease in the fiber length compared to those in normal corneas, suggesting that standard treatments against DED (lubrication or topical cyclosporine) do not protect significantly against nerve damage. Similarly, IL-1Ra and anti-IL17 therapies maintain the nerve fiber tortuosity levels similar to that seen in normal corneas, whereas DED corneas treated with vehicle or CsA show ˜2-fold increase in the nerve fiber tortuosity as compared to the normals (FIG. 8C).

These results demonstrate that blockade of Interleukin-1 (IL-1) either directly using topical IL-1 receptor antagonist (IL-1Ra) or indirectly by blocking IL-17 (a potent inducer of IL-1β by target cells) using anti-IL17-antibody inhibits corneal neuropathy, including the symptoms of nerve loss (fiber length) and nerve tortuosity. The treatments that block IL-1 and IL-17 promote nerve fiber length, but suppress nerve fiber tortuosity.

The nerve tortuosity data and the nerve fiber length data described above indicate that conventional therapies for DED (topical lubricating tear ointment or topical cyclosporine-A) do not protect significantly against nerve damage. Although ocular surface inflammation associated with DED is often correlated with elevated levels of tear nerve growth factor, which is typically reduced with steroids such as 0.1% prednisolone, the results above indicate that such therapy are ineffective at inducing corneal nerve regeneration. Thus, administration of such immunosuppressive agents (e.g., macrolides such as cyclosporin A (e.g., Restasis®), or corticosteroids such as prednisolone) are preferably not administered with the compositions described herein.

### Example 7

Combination therapy blockade of IL-1 and IL-17 enhances corneal nerve regeneration IL-1 is a major initiator of the inflammatory process upstream of T cell activation and plays a crucial role in precipitating the inflammation by promoting the induction or expansion of IL-17-secreting T cells. IL-1 and IL-17 work synergistically and enhance secretion of each other in vivo.

An IL-17 blocking treatment is applied to a cornea, along with an IL-1 blocking treatment. The co-administration of IL-1 and IL-17 blocking treatment enhances corneal nerve regeneration such that the amount of nerve fibers associated with damaged corneas is increased compared to untreated corneas. The co-administration of IL-1 and IL-17 blocking treatment synergistically enhances corneal nerve regeneration in the case of immune-mediated corneal nerve damage.

### Example 8

### Efficacy of Topical Application of IL-1Ra in Ocular Surface Inflammatory Disorders in a Human

An IL-1Ra open label study was utilized to determine the efficacy of IL-1Ra in treating ocular surface inflammatory disorders in a human. Seventy patients were enrolled in the study and various numbers of patients were available for monthly checkups as follows: month 1 (44 patients), month 3 (37 patients), month 5 (22 patients), and month 7 (17 patients). None of these patients had severe meibomian gland dysfunction (MGD, also called posterior blepharitis) defined as grade 3 meibomian secretions (secretions retain shape after expression), or grade 4 lid margin disease (marked diffuse redness of both lid margins and skin), or grade 4 conjunctival hyperemia (marked dark redness of the palpebral and/or bulbar conjunctiva). Of note, patients with MGD/meibomian gland dysfunction alone do not develop significant corneal neuropathy (Foulks and Bron, 2003 Ocul Surf, 107-26).

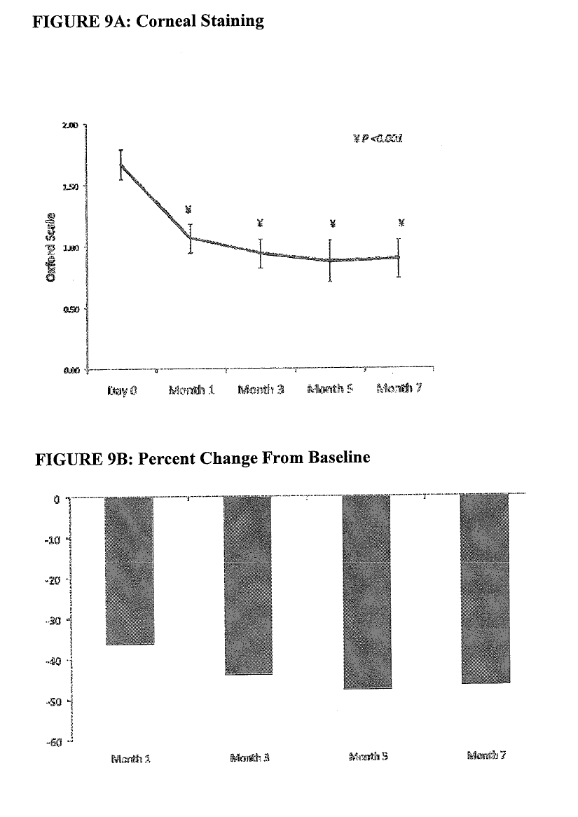

As shown in FIGS. 9A and 9B, corneal staining was utilized to measure ocular surface disease treated with topical IL-1 receptor antagonist. Specifically, the application of fluorescein was used to detect epithelial damage in the cornea, and each patient's disease severity was graded using the Oxford scale. The data demonstrate a significant reduction in staining (severity of disease) with topical blockade of IL-1 using the IL-1 receptor antagonist. Specifically, a significant change was observed from baseline to 7 months follow-up after treatment with IL-1 receptor antagonist.

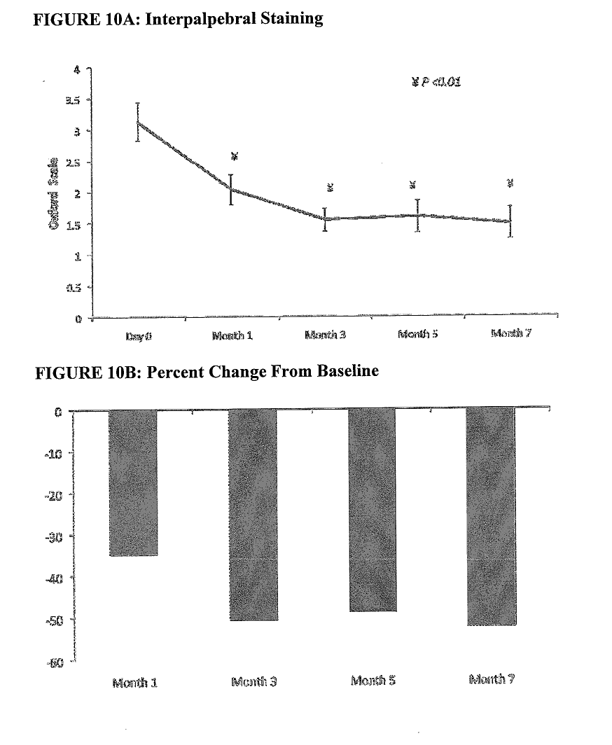

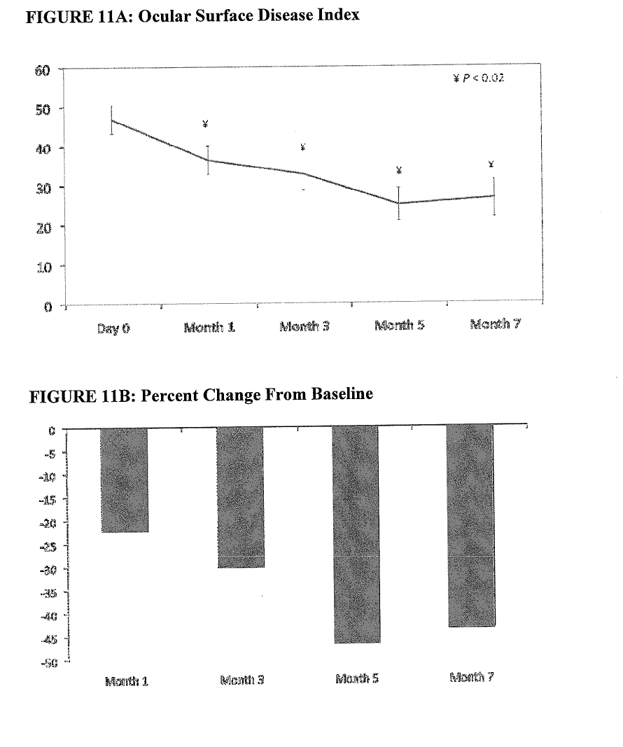

As depicted in FIGS. 10A and 10B, lisamine green staining was utilized to detect damage to the conjunctival epithelium, and each patient's disease was graded using the standardized Oxford scale. A significant reduction in interpalpebral (conjunctival) staining (severity of disease) was observed from baseline to 7 months follow-up after treatment with IL-1 receptor antagonist. Ocular surface disease index (OSDI) is a validated instrument for measuring dry eye disease-related ocular surface disease symptoms' severity and effect on vision-related function. The score ranges from 0 (no symptoms) to 100 (maximal symptoms and visual dysfunction in all categories), with higher scores representing greater disability. OSDI was determined for each patient treated with IL-1 receptor antagonist (FIGS. 11A and 11B). OSDI score decreased significantly from baseline to 7 months follow-up after treatment with IL-1 receptor antagonist. In addition, a significant number of patients who experienced ocular pain reported a reduction in pain soon after topical administration of IL-1 receptor antagonist. In some cases, reduction in pain can be experienced, e.g., within minutes. Since pain-sensing nerves express IL-1 receptor, we believe that the reduction of pain after topical administration of the IL-1 antagonist is not only due to improvements in the surface epithelium health as a result of IL-1 inhibition but also a direct effect on the corneal/ocular surface nerves by inhibiting the ability of inflammatory IL-1 cytokines to activate IL-1 receptor on these nerves. Accordingly, inhibitors of IL-1 receptor can be used to reduce pain and can lead to reductions in discomfort (e.g., by a reduction of 2, 3, 5, 7 or more in discomfort scores as evaluated by in the ocular discomfort module of the OSDI also referred to as “OSDI-symptoms”).

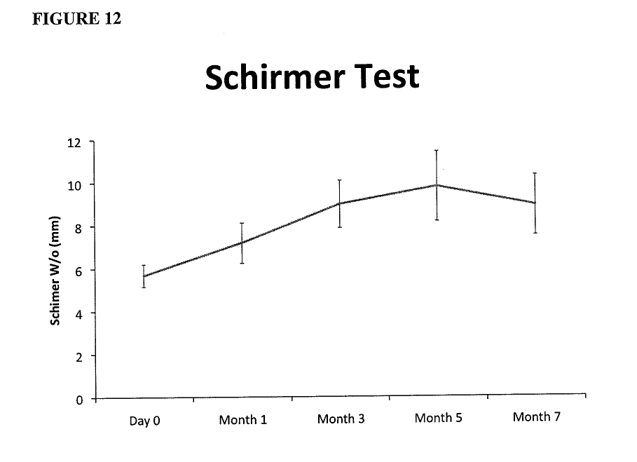

The Schirmer test measures the amount of tear produced by a patient's eye. The test is performed by placing a special scaled filter paper strip between the lower eyelid and the eyeball for 5 minutes. As shown in FIG. 12, tear volume increased from below 6 mm at baseline to ˜9 mm at 7 months of follow-up. The data suggest an overall trend toward higher tear secretion with topical blockade of IL-1 with IL-1 receptor antagonist.

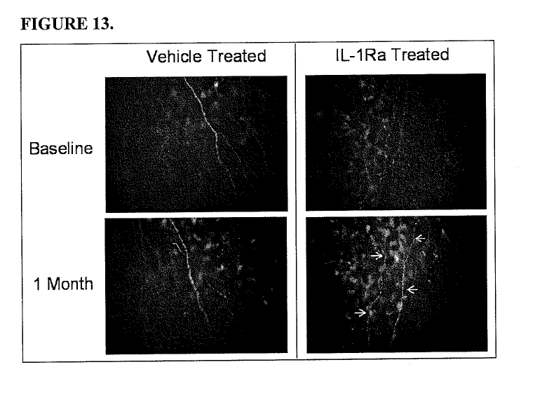

FIG. 13 shows confocal micrographs of corneas from patients with dry eye disease before and after treatment with topical vehicle or topical IL-1Ra. One month of IL-Ra treatment promoted corneal nerve regeneration (marked with white arrows). By contrast, one month of vehicle treatment (lubricating drops) did not promote corneal nerve regeneration. These data support the observation made in mice (above) that selective blockade of cytokines with pathogenic roles in dry eye disease (IL-1, IL-17) provide a level of neuroprotection not observed with standard treatments.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. Genbank and NCBI submissions indicated by accession number cited herein are hereby incorporated by reference. All other published references, documents, manuscripts and scientific literature cited herein are hereby incorporated by reference.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for protecting or regenerating corneal nerves in a subject in need thereof, comprising the steps of:
(a) identifying a subject with corneal nerve damage or loss; and
(b) locally administering to the cornea of said subject a composition that inhibits an activity of an inflammatory interleukin-1 cytokine, thereby enhancing corneal nerve regeneration and reducing the development of abnormalities in nerve morphology or density.

2. The method of claim 1, wherein said subject is identified as having corneal nerve damage or loss that results from a congenital defect, disease, trauma, medical or surgical procedure.

3. The method of claim 1, wherein said subject is identified as having corneal nerve damage or loss that results from neurotrophic keratitis, herpes simplex, zoster keratitis, diabetes mellitus, trigeminal nerve damage, ocular or orbital or head surgery, head trauma, aneurysm, intracranial neurologic disease, keratorefractive procedures, photorefractive keratectomy (PRK), laser in situ keratomileusis (LASIK), congenital defect, ocular surface disease, dry eye syndrome, or peripheral neuropathy.

4. The method of claim 8, wherein the subject exhibits a decrease of corneal innervation or sensation, a reduction in the number of nerve fibers or bundles innervating the cornea, death of neurons innervating the cornea, a decrease or loss of neurotransmitter release, a decrease or loss of nerve growth factor release, abnormal tearing reflexes, abnormal blink reflexes, abnormal nerve morphology, appearance of abnormal nerve sprouts, abnormal tortuosity, increased bead-like nerve formations, thinning of nerve fiber bundles, or thickening of nerve fiber bundles.

5. The method of claim 1, wherein said composition is a protein that inhibits binding of an inflammatory IL-1 cytokine to an IL-1 receptor.

6. The method of claim 5, wherein said protein binds to the Interleukin-1 receptor, type I (IL-1RI).

7. The method of claim 5, wherein said protein comprises an amino acid sequence of SEQ ID NO: 16 or a sequence at least 90% identical to SEQ ID NO:16.

8. The method of claim 5, wherein said protein comprises an antibody that binds to IL-1α, IL-1β or IL-1RI.

9. The method of claim 1, wherein said composition is present in a concentration of 0.1-10% (mg/ml).

10. The method of claim 1, wherein said composition is present in a concentration of 2.5% (mg/ml) or 5% (mg/ml).

11. The method of claim 1, wherein said composition is administered topically.

12. The method of claim 1, wherein said method does not comprise systemic administration or substantial dissemination of the composition to non-ocular tissue.

13. The method of claim 1, wherein said composition further comprises a compound selected from the group consisting of a physiological acceptable salt, poloxamer analogs with carbopol, carbopol/HPMC, carbopol-methyl cellulose, a mucolytic agent, carboxymethylcellulose (CMC), hyaluronic acid, cyclodextrin, and petroleum.

14. The method of claim 1, wherein said composition further comprises carboxymethylcellulose (CMC).

15. The method of claim 1, further comprising locally administering an antagonist of interleukin-17 or a receptor thereof.

16. A method for protecting or regenerating corneal nerves in a subject in need thereof, comprising the steps of:
(a) identifying a subject with corneal nerve damage or loss; and
(b) locally administering to the cornea of said subject a composition that inhibits an activity of an inflammatory interleukin-1 cytokine and a composition that inhibits an activity of an inflammatory interleukin-17 cytokine, thereby enhancing corneal nerve regeneration and reducing the development of abnormalities in nerve morphology or density.

17. A method of reducing ocular pain in a subject in need thereof, the method comprising:
topically administering, to a subject experiencing ocular pain, a protein that inhibits binding of an inflammatory IL-1 cytokine to an IL-1 receptor in an amount effective reduce ocular pain.

18. The method of claim 17, wherein said protein comprises an amino acid sequence of anakinra or SEQ ID NO: 16 or a sequence at least 90% identical to anakinra or SEQ ID NO:16.

19. The method of claim 17, wherein said protein comprises an antibody that binds to IL-1α, IL-1β or IL-1RI.

20. The method of claim 18, wherein said protein is present in a concentration of 0.1-10% (mg/ml).

21. The method of claim 18, wherein said protein is present in a concentration of 2.5% (mg/ml) or 5% (mg/ml).

22. The method of claim 17 wherein the protein is delivered in an eye drop.


SEQUENCE LISTING (45 sequences)

SEQ ID NO: 1 - DNA, 2943 bp, Homo sapiens
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 2 - protein, 213 aa, Homo sapiens
MAKVPDMFEDLKNCYSENEEDSSSIDHLSLNQKSFYHVSYGPLHDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 3 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 4 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 5 - DNA, 1760 bp, Homo sapiens
atttctttataaaccacaactctgggcccgcaatggcagtccactgccttgctgcagtcacagaatggaaatctgcagaggcctccgcagtcacctaatcactctcctcctcttcctgttccattcagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaa

SEQ ID NO: 6 - protein, 177 aa, Homo sapiens
MEICRGLRSHLITLLLFLFHSETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 7 - DNA, 1865 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagctgacttgtatgaagaaggaggtggaggaggaggagaaggtgaagacaatgctgactcaaaggagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 8 - protein, 180 aa, Homo sapiens
MALADLYEEGGGGGGEGEDNADSKETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 9 - DNA, 1802 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 10 - protein, 159 aa, Homo sapiens
MALETICRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 11 - DNA, 1973 bp, Homo sapiens
gggcagctccaccctgggagggactgtggcccaggtactgcccgggtgctactttatgggcagcagctcagttgagttagagtctggaagacctcagaagacctcctgtcctatgaggccctccccatggctttagggggattataaaactaatcatcaaagccaagaaggcaagagcaagcatgtaccgctgaaaacacaagataactgcataagtaatgactttcagtgcagattcatagctaacccataaactgctggggcaaaaatcatcttggaaggctctgaacctcagaaaggattcacaagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagcggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtagtactgcccaggcctgcctgttcccattcttgcatggcaaggactgcagggactgccagtccccctgccccagggctcccggctatgggggcactgaggaccagccattgaggggtggaccctcagaaggcgtcacaagaacctggtcacaggactctgcctcctcttcaactgaccagcctccatgctgcctccagaatggtctttctaatgtgtgaatcagagcacagcagcccctgcacaaagcccttccatgtcgcctctgcattcaggatcaaaccccgaccacctgcccaacctgctctcctcttgccactgcctcttcctccctcattccaccttcccatgccctggatccatcaggccacttgatgacccccaaccaagtggctcccacaccctgttttacaaaaaagaaaagaccagtccatgagggaggtttttaagggtttgtggaaaatgaaaattaggatttcatgatttttttttttcagtccccgtgaaggagagcccttcatttggagattatgttctttcggggagaggctgaggacttaaaatattcctgcatttgtgaaatgatggtgaaagtaagtggtagcttttcccttctttttcttctttttttgtgatgtcccaacttgtaaaaattaaaagttatggtactatgttagccccataattttttttttccttttaaaacacttccataatctggactcctctgtccaggcactgctgcccagcctccaagctccatctccactccagattttttacagctgcctgcagtactttacctcctatcagaagtttctcagctcccaaggctctgagcaaatgtggctcctgggggttctttcttcctctgctgaaggaataaattgctccttgacattgtagagcttctggcacttggagacttgtatgaaagatggctgtgcctctgcctgtctcccccaccgggctgggagctctgcagagcaggaaacatgactcgtatatgtctcaggtccctgcagggccaagcacctagcctcgctcttggcaggtactcagcgaatgaatgctgtatatgttgggtgcaaagttccctacttcctgtgacttcagctctgttttacaataaaatcttgaaaatgcctaaaaaaaaaaaaaaaaaaaaaaaaaaaaaaaaaaaaaaaaaaaaaaaaaaaa

SEQ ID NO: 12 - protein, 143 aa, Homo sapiens
MQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 13 - DNA, 602 bp, Homo sapiens
agctccaccctgggagggactgtggcccaggtactgcccgggtgctactttatgggcagcagctcagttgagttagagtctggaagacctcagaagacctcctgtcctatgaggccctccccatggctttagagacgatctgccgaccctctgggagaaaatccagcaagatgcaagccttcagaatctgggatgttaaccagaagaccttctatctgaggaacaaccaactagttgctggatacttgcaaggaccaaatgtcaatttagaagaaaagatagatgtggtacccattgagcctcatgctctgttcttgggaatccatggagggaagatgtgcctgtcctgtgtcaagtctggtgatgagaccagactccagctggaggcagttaacatcactgacctgagcgagaacagaaagcaggacaagcgcttcgccttcatccgctcagacagtggccccaccaccagttttgagtctgccgcctgccccggttggttcctctgcacagcgatggaagctgaccagcccgtcagcctcaccaatatgcctgacgaaggcgtcatggtcaccaaattctacttccaggaggacgagtag

SEQ ID NO: 14 - protein, 159 aa, Homo sapiens; identical to SEQ ID NO: 10

SEQ ID NO: 15 - DNA, 602 bp, Homo sapiens; identical to SEQ ID NO: 13

SEQ ID NO: 16 - protein, 153 aa, Homo sapiens
MRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 17 - DNA, 4909 bp, Homo sapiens
tagacgcaccctctgaagatggtgactccctcctgagaagctggaccccttggtaaaagacaaggccttctccaagaagaatatgaaagtgttactcagacttatttgtttcatagctctactgatttcttctctggaggctgataaatgcaaggaacgtgaagaaaaaataattttagtgtcatctgcaaatgaaattgatgttcgtccctgtcctcttaacccaaatgaacacaaaggcactataacttggtataaagatgacagcaagacacctgtatctacagaacaagcctccaggattcatcaacacaaagagaaactttggtttgttcctgctaaggtggaggattcaggacattactattgcgtggtaagaaattcatcttactgcctcagaattaaaataagtgcaaaatttgtggagaatgagcctaacttatgttataatgcacaagccatatttaagcagaaactacccgttgcaggagacggaggacttgtgtgcccttatatggagttttttaaaaatgaaaataatgagttacctaaattacagtggtataaggattgcaaacctctacttcttgacaatatacactttagtggagtcaaagataggctcatcgtgatgaatgtggctgaaaagcatagagggaactatacttgtcatgcatcctacacatacttgggcaagcaatatcctattacccgggtaatagaatttattactctagaggaaaacaaacccacaaggcctgtgattgtgagcccagctaatgagacaatggaagtagacttgggatcccagatacaattgatctgtaatgtcaccggccagttgagtgacattgcttactggaagtggaatgggtcagtaattgatgaagatgacccagtgctaggggaagactattacagtgtggaaaatcctgcaaacaaaagaaggagtaccctcatcacagtgcttaatatatcggaaattgaaagtagattttataaacatccatttacctgttttgccaagaatacacatggtatagatgcagcatatatccagttaatatatccagtcactaatttccagaagcacatgattggtatatgtgtcacgttgacagtcataattgtgtgttctgttttcatctataaaatcttcaagattgacattgtgctttggtacagggattcctgctatgattttctcccaataaaagcttcagatggaaagacctatgacgcatatatactgtatccaaagactgttggggaagggtctacctctgactgtgatatttttgtgtttaaagtcttgcctgaggtcttggaaaaacagtgtggatataagctgttcatttatggaagggatgactacgttggggaagacattgttgaggtcattaatgaaaacgtaaagaaaagcagaagactgattatcattttagtcagagaaacatcaggcttcagctggctgggtggttcatctgaagagcaaatagccatgtataatgctcttgttcaggatggaattaaagttgtcctgcttgagctggagaaaatccaagactatgagaaaatgccagaatcgattaaattcattaagcagaaacatggggctatccgctggtcaggggactttacacagggaccacagtctgcaaagacaaggttctggaagaatgtcaggtaccacatgccagtccagcgacggtcaccttcatctaaacaccagttactgtcaccagccactaaggagaaactgcaaagagaggctcacgtgcctctcgggtagcatggagaagttgccaagagttctttaggtgcctcctgtcttatggcgttgcaggccaggttatgcctcatgctgacttgcagagttcatggaatgtaactatatcatcctttatccctgaggtcacctggaatcagattattaagggaataagccatgacgtcaatagcagcccagggcacttcagagtagagggcttgggaagatcttttaaaaaggcagtaggcccggtgtggtggctcacgcctataatcccagcactttgggaggctgaagtgggtggatcaccagaggtcaggagttcgagaccagcccagccaacatggcaaaaccccatctctactaaaaatacaaaaatgagctaggcatggtggcacacgcctgtaatcccagctacacctgaggctgaggcaggagaattgcttgaaccggggagacggaggttgcagtgagccgagtttgggccactgcactctagcctggcaacagagcaagactccgtctcaaaaaaagggcaataaatgccctctctgaatgtttgaactgccaagaaaaggcatggagacagcgaactagaagaaagggcaagaaggaaatagccaccgtctacagatggcttagttaagtcatccacagcccaagggcggggctatgccttgtctggggaccctgtagagtcactgaccctggagcggctctcctgagaggtgctgcaggcaaagtgagactgacacctcactgaggaagggagacatattcttggagaactttccatctgcttgtattttccatacacatccccagccagaagttagtgtccgaagaccgaattttattttacagagcttgaaaactcacttcaatgaacaaagggattctccaggattccaaagttttgaagtcatcttagctttccacaggagggagagaacttaaaaaagcaacagtagcagggaattgatccacttcttaatgctttcctccctggcatgaccatcctgtcctttgttattatcctgcattttacgtctttggaggaacagctccctagtggcttcctccgtctgcaatgtcccttgcacagcccacacatgaaccatccttcccatgatgccgctcttctgtcatcccgctcctgctgaaacacctcccaggggctccacctgttcaggagctgaagcccatgctttcccaccagcatgtcactcccagaccacctccctgccctgtcctccagcttcccctcgctgtcctgctgtgtgaattcccaggttggcctggtggccatgtcgcctgcccccagcactcctctgtctctgctcttgcctcgacccttcctcctcctttgcctaggaggccttctcgcattttctctagctgatcagaattttaccaaaattcagaacatcctccaattccacagtctctgggagactttccctaagaggcgacttcctctccagccttctctctctggtcaggcccactgcagagatggtggtgagcacatctgggaggctggtctccctccagctggaattgctgctctctgagggagaggctgtggtggctgtctctgtccctcactgccttccaggagcaatttgcacatgtaacatagatttatgtaatgctttatgtttaaaaacattccccaattatcttatttaatttttgcaattattctaattttatatatagagaaagtgacctattttttaaaaaaatcacactctaagttctattgaacctaggacttgagcctccatttctggcttctagtctggtgttctgagtacttgatttcaggtcaataacggtcccccctcactccacactggcacgtttgtgagaagaaatgacattttgctaggaagtgaccgagtctaggaatgcttttattcaagacaccaaattccaaacttctaaatgttggaattttcaaaaattgtgtttagattttatgaaaaactcttctactttcatctattctttccctagaggcaaacatttcttaaaatgtttcattttcattaaaaatgaaagccaaatttatatgccaccgattgcaggacacaagcacagttttaagagttgtatgaacatggagaggacttttggtttttatatttctcgtatttaatatgggtgaacaccaacttttatttggaataataattttcctcctaaacaaaaacacattgagtttaagtctctgactcttgcctttccacctgctttctcctgggcccgctttgcctgcttgaaggaacagtgctgttctggagctgctgttccaacagacagggcctagctttcatttgacacacagactacagccagaagcccatggagcagggatgtcacgtcttgaaaagcctattagatgttttacaaatttaattttgcagattattttagtctgtcatccagaaaatgtgtcagcatgcatagtgctaagaaagcaagccaatttggaaacttaggttagtgacaaaattggccagagagtgggggtgatgatgaccaagaattacaagtagaatggcagctggaatttaaggagggacaagaatcaatggataagcgtgggtggaggaagatccaaacagaaaagtgcaaagttattccccatcttccaagggttgaattctggaggaagaagacacattcctagttccccgtgaacttcctttgacttattgtccccactaaaacaaaacaaaaaacttttaatgccttccacattaattagattttcttgcagtttttttatggcatttttttaaagatgccctaagtgttgaagaagagtttgcaaatgcaacaaaatatttaattaccggttgttaaaactggtttagcacaatttatattttccctctcttgcctttcttatttgcaataaaaggtattgagccattttttaaatgacatttttgataaattatgtttgtactagttgatgaaggagttttttttaacctgtttatataattttgcagcagaagccaaattttttgtatattaaagcaccaaattcatgtacagcatgcatcacggatcaatagactgtacttattttccaataaaattttcaaactttgtactgttaaa

SEQ ID NO: 18 - protein, 569 aa, Homo sapiens
MKVLLRLICFIALLISSLEADKCKEREEKIILVSSANEIDVRPCPLNPNEHKGTITWYKDDSKTPVSTEQASRIHQHKEKLWFVPAKVEDSGHYYCVVRNSSYCLRIKISAKFVENEPNLCYNAQAIFKQKLPVAGDGGLVCPYMEFFKNENNELPKLQWYKDCKPLLLDNIHFSGVKDRLIVMNVAEKHRGNYTCHASYTYLGKQYPITRVIEFITLEENKPTRPVIVSPANETMEVDLGSQIQLICNVTGQLSDIAYWKWNGSVIDEDDPVLGEDYYSVENPANKRRSTLITVLNISEIESRFYKHPFTCFAKNTHGIDAAYIQLIYPVTNFQKHMIGICVTLTVIIVCSVFIYKIFKIDIVLWYRDSCYDFLPIKASDGKTYDAYILYPKTVGEGSTSDCDIFVFKVLPEVLEKQCGYKLFIYGRDDYVGEDIVEVINENVKKSRRLIIILVRETSGFSWLGGSSEEQIAMYNALVQDGIKVVLLELEKIQDYEKMPESIKFIKQKHGAIRWSGDFTQGPQSAKTRFWKNVRYHMPVQRRSPSSKHQLLSPATKEKLQREAHVPLG

SEQ ID NO: 19 - DNA, 1484 bp, Homo sapiens
cccgtgaggaggaaaaggtgtgtccgctgccacccagtgtgagcgggtgacaccacccggttaggaaatcccagctcccaagagggtataaatccctgctttactgctgagctcctgctggaggtgaaagtctggcctggcagccttccccaggtgagcagcaacaaggccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaaaaaaaaaa

SEQ ID NO: 20 - DNA, 1436 bp, Homo sapiens
gggatgggagatactgttgtggtcacctctggaaaatacattctgctactcttaaaaactagtgacgctcatacaaatcaacagaaagagcttctgaaggaagactttaaagctgcttctgccacgtgctgctgggtctcagtcctccacttcccgtgtcctctggaagttgtcaggagcaatgttgcgcttgtacgtgttggtaatgggagtttctgccttcacccttcagcctgcggcacacacaggggctgccagaagctgccggtttcgtgggaggcattacaagcgggagttcaggctggaaggggagcctgtagccctgaggtgcccccaggtgccctactggttgtgggcctctgtcagcccccgcatcaacctgacatggcataaaaatgactctgctaggacggtcccaggagaagaagagacacggatgtgggcccaggacggtgctctgtggcttctgccagccttgcaggaggactctggcacctacgtctgcactactagaaatgcttcttactgtgacaaaatgtccattgagctcagagtttttgagaatacagatgctttcctgccgttcatctcatacccgcaaattttaaccttgtcaacctctggggtattagtatgccctgacctgagtgaattcacccgtgacaaaactgacgtgaagattcaatggtacaaggattctcttcttttggataaagacaatgagaaatttctaagtgtgagggggaccactcacttactcgtacacgatgtggccctggaagatgctggctattaccgctgtgtcctgacatttgcccatgaaggccagcaatacaacatcactaggagtattgagctacgcatcaagaaaaaaaaagaagagaccattcctgtgatcatttcccccctcaagaccatatcagcttctctggggtcaagactgacaatcccgtgtaaggtgtttctgggaaccggcacacccttaaccaccatgctgtggtggacggccaatgacacccacatagagagcgcctacccgggaggccgcgtgaccgaggggccacgccaggaatattcagaaaataatgagaactacattgaagtgccattgatttttgatcctgtcacaagagaggatttgcacatggattttaaatgtgttgtccataataccctgagttttcagacactacgcaccacagtcaaggaagcctcctccacgttctcctggggcattgtgctggccccactttcactggccttcttggttttggggggaatatggatgcacagacggtgcaaacacagaactggaaaagcagatggtctgactgtgctatggcctcatcatcaagactttcaatcctatcccaagtgaaataaatggaatgaaataattcaaacacaaaaaaaaaaaaaaaaaaaaaaaaaaaaaa

SEQ ID NO: 21 - protein, 398 aa, Homo sapiens
MLRLYVLVMGVSAFTLQPAAHTGAARSCRFRGRHYKREFRLEGEPVALRCPQVPYWLWASVSPRINLTWHKNDSARTVPGEEETRMWAQDGALWLLPALQEDSGTYVCTTRNASYCDKMSIELRVFENTDAFLPFISYPQILTLSTSGVLVCPDLSEFTRDKTDVKIQWYKDSLLLDKDNEKFLSVRGTTHLLVHDVALEDAGYYRCVLTFAHEGQQYNITRSIELRIKKKKEETIPVIISPLKTISASLGSRLTIPCKVFLGTGTPLTTMLWWTANDTHIESAYPGGRVTEGPRQEYSENNENYIEVPLIFDPVTREDLHMDFKCVVHNTLSFQTLRTTVKEASSTFSWGIVLAPLSLAFLVLGGIWMHRRCKHRTGKADGLTVLWPHHQDFQSYPK

SEQ ID NO: 22 - DNA, 3177 bp, Homo sapiens
cagaagacctcctgtcctatgaggccctccccatggctttaggtaagctccttccactctcattttttcacctgagaaatgagagaggaaaatgtctacaattggtgtttatcaaatgctttcaggctctggtgagcaagcgtccaggaaaatgtcaagcgcatggagctccaggcctgtctgggggatctgggcacggggaggcatccatgggagaccatgcaggcactctgaggcaggggctgcaagctagtgcctgctggggcagcaggtgaacagagaggtgtaactgctgtgacagaagtcatggagtccttggagtgtgagggtcattttccactgttgatagaatagggaaattggtgaaatagccctgttaaatgagagaaagaacagtgtgagctcaatgagaaatactaatagaatgtggcactgagccacaaggtctgagggttgattgataaggaagggtggggactgtggagaattaagggcttggcacaggtcagttccaccagttgtcacaagagaatgcaggctcaggtggccagaacttctcgcttttccagaagagtccgatattctgatttcattatatatagtattctgattaaaccagacaataaagcaagcagataaaatatttaaagtataagctgccagtttgcaacctccggttaggatttgtgtggggcaaagaaaaaaactctcaggatcattggtatgtagactctaattttaagtttctaatttaaaattggcccctgaggctgggcgtggtggctcacacctgtaatcccagcattttgggaggccaaggtgggtggatctcttgaggtcaagagttcaaggcctgcctggccaacatggtgaaaccctgtctctattaaaaatacaaaaattagctgggcatggtggtgcatgtctgcaatcttagctacttgggtagctaaggcaggagaattgctggaacccgggaggtagaggttgcagtgaatggagatcacaccactgcactccagtctgggcaatagagagagacgctctctctaaaaaaaaatatgtaaagataaataaaatgaaataaaataggcctctaatgagcaggccattctcctttctgggtcttactttccttgcactcctttctgggtgttaagaggaggtctagaggaagctggacaactcttagcttgtagtaagcacagtggaagtatcagctcttaatgggtcatggacacgttacgaagctaggcgccgtgctgagcactttacatggtttatcccactgaaccctctcaataaccctatgaggaagggctattattgctcacattttcagaagaggaaatggatatagagagattagataatttgcccatggccagacagctagtataagaggaggaggtggattgactgcagacattctgtcttcaaaccactacactatgctatggaggcacagagacttaatgaaatcatggagaggggaattgctttgtcaaccacaagcagttattccgggggcagcagatcctcccctgtcccccagtggtacaatggtccctggtgggttgtgctacaatgttagcccatggtcttatgtgtttttcaaatgtgtaaagtaggatgctggaaccactcttagaaccagataccaatacattgtgaagaaataaatctctgtgcttaaaactggttcatcccaaaatattttgaactgacacacaataggtgctaaataaatgtgtgttaacttgaattggattgaattcgggaaaaaagtgcaataagcttagtgaagacaccatgttccctgggtagaggaaccacattctccatctaaggccaggagtatgggaggtatcaatgtttgcccagcacagaacagggtgccaagaagagaaaagttgacggggtgcatactctgactggaaactggaagggtgagaacagagggtaaaggatagagatggaaccatgtgcatacactttgtgttaccttggacaagtcattcatttctctggacctctgctttctctctacacaatggggtcccaccacttcccttacagctgacttgtatgaagaaggaggtggaggaggaggagaaggtgaagacaatgctgactcaaagggtaaattatttttaggatccaagtttgaaaacaattttaggctactagatatgaacaacatcttgattatgtagttgaaggaaattaaagatgaatggtttaattaaaaattaatcagaatgaaaacgattgattactaatatatctgcaatggtttattttcctgagtggcagactcactaaggtttttgaatactcctgtgtgattgctctatgtatgtatgtatgtatgtatgtatgcatgtatctatctatctgttgtctaatagaatggatcacatctctgctaataaaaacactacactggcagggtacaattataatcattaactgtgcctggaatttgcagcagcagccaccagaggtaccagtgccctttaagggttcataatttagaataatccaattatctgagtttttcagggactgaggggtttggcaaggtgtagaactttcagtaataaagtcaagaaagtcctggacaaaccaaggtagttggtcactctagtccataaccaggtaaagagctttccctgtaacctgtgtaaggttttagaatcatttctttccttattaccaaaaatcctccccaaattttcaagaaattatgaactaaatagttactctatgagataggagttcagcccaaaagaaacaccataagaacaaatataattcttgcttatgttaaccatgcaatgaagcagagagaaaaagtcagtggcctctttaggaggactgtagtgtgggaagaaataactaaactgggtttcaatcctggcctggccaggatctggagcaagtgagttaatctttctaagccttgagtagtttataaaagaatggccactccatagacagagtagcctgaaccttgagttcttctataaagtcactatgaatttatactcattttgaaagtgggtgtcaatatgtctgtccactttgcacagctgttatgtggacaaaaggagatctgtgtgaaagtgtaacacagagcctaaactataacaggtaagcaacacagttgtccct

SEQ ID NO: 23 - protein, 20 aa, Homo sapiens
DLYEEGGGGGGEGEDNADSK

SEQ ID NO: 24 - DNA, 4725 bp, Homo sapiens
tgccgggatccaggtctccggggtccgctttggccagaggcgcggaaggaagcagtgcccggcgacactgcacccatcccggctgcttttgctgcgccctctcagcttcccaagaaaggcatcgtcatgtgatcatcacctaagaactagaacatcagcaggccctagaagcctcactcttgcccctccctttaatatctcaaaggatgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttccaaagcagccaaggtgaagcagaaagtgccagctccaagatacacagtggaactggcttgtggttttggagccacagtcctgctagtggtgattctcattgttgtttaccatgtttactggctagagatggtcctattttaccgggctcattttggaacagatgaaaccattttagatggaaaagagtatgatatttatgtatcctatgcaaggaatgcggaagaagaagaatttgtattactgaccctccgtggagttttggagaatgaatttggatacaagctgtgcatctttgaccgagacagtctgcctgggggaattgtcacagatgagactttgagcttcattcagaaaagcagacgcctcctggttgttctaagccccaactacgtgctccagggaacccaagccctcctggagctcaaggctggcctagaaaatatggcctctcggggcaacatcaacgtcattttagtacagtacaaagctgtgaaggaaacgaaggtgaaagagctgaagagggctaagacggtgctcacggtcattaaatggaaaggggaaaaatccaagtatccacagggcaggttctggaagcagctgcaggtggccatgccagtgaagaaaagtcccaggcggtctagcagtgatgagcagggcctctcgtattcatctttgaaaaatgtatgaaaggaataatgaaaagggtaaaaagaacaaggggtgctccaggaagaaagagtccccccagtcttcattcgcagtttatggtttcataggcaaaaataatggtctaagcctcccaatagggataaatttagggtgactgtgtggctgactattctgcttcctcaggcaacactaaagtttagaaagatatcatcaacgttctgtcaccagtctctgatgccactatgttctttgcaggcaaagacttgttcaatgcgaatttccccttctacattgtctatccctgtttttatatgtctccattctttttaaaatcttaacatatggagcagcctttcctatgaatttaaatatgcctttaaaataagtcactgttgacagggtcatgagtttccgagtatagttttctttttatcttatttttactcgtccgttgaaaagataatcaaggcctacattttagctgaggataatgaacttttttcctcattcggctgtataatacataaccacagcaagactgacatccacttaggatgatacaaagcagtgtaactgaaaatgtttcttttaattgatttaaaggacttgtcttctataccacccttgtcctcatctcaggtaatttatgaaatctatgtaaacttgaaaaatatttcttaatttttgtttttgctccagtcaattcctgattatccacaggtcaacccacattttttcattccttctccctatctgcttatatcgcattgctcatttagagtttgcaggaggctccatactaggttcagtctgaaagaaatctcctaatggtgctatagagagggaggtaacagaaagactcttttagggcatttttctgactcatgaaaagagcacagaaaaggatgtttggcaatttgtcttttaagtcttaaccttgctaatgtgaatactgggaaagtgattttttctcactcgtttttgttgctccattgtaaagggcggaggtcagtcttagtggccttgagagttgcttttggcattaatattctaagagaattaactgtatttcctgtcacctattcactagtgcaggaaatatacttgctccaaataagtcagtatgagaagtcactgtcaatgaaagttgttttgtttgttttcagtaatattttgctgtttttaagacttggaaaactaagtgcagagtttacagagtggtaaatatctatgttacatgtagattatacatatatatacacacgtgtatatgagatatatatcttatatctccacaaacacaaattatatatatacatatccacacacatacattacatatatctgtgtatataaatccacatgcacatgaaatatatatatatatataatttgtgtgtgtgtatgtgtatgtatatgactttaaatagctatgggtacaatattaaaaaccactggaactcttgtccagtttttaaattatgtttttactggaatgtttttgtgtcagtgttttctgtacatattatttgttaattcacagctcacagagtgatagttgtcatagttcttgccttccctaagtttatataaataacttaagtattgctacagtttatctaggttgcagtggcatctgctgtgcacagagcttccatggtcactgctaagcagtagccagccatcgggcattaattgatttcctactatattcccagcagacacatttagaaactaagctatgttaacctcagtgctcaactatttgaactgttgagtgataaaggaaacaaatataactgtaaatgaatcttggtatcctgtgaaacagaataattcgtaatttaagaaagcccttatcccggtaacatgaatgttgatgaacaaatgtaaaattatatcctatatttaagtacccataataaatcatttccctctataagtgttattgattattttaaattgaaaaaagtttcacttggatgaaaaaagtagaaaagtaggtcattcttggatctacttttttttagccttattaatatttttccctattagaaaccacaattactccctctattaacccttcacttactagaccagaaaagaacttattccagataagctttgaatatcaattcttacataaactttaggcaaacagggaatagtctagtcaccaaaggaccattctcttgccaatgctgcattccttttgcacttttggattccatatttatcccaaatgctgttgggcacccctagaaataccttgatgttttttctatttatatgcctgcctttggtacttaattttacaaatgctgtaatataaagcatatcaagtttatgtgatacgtatcattgcaagagaatttgtttcaagatttttttttaatgttccagaagatggccaatagagaacattcaagggaaatggggaaacataatttagagaacaagaacaaaccatgtctcaaatttttttaaaaaaaattaatggttttaaatatatgctatagggacgttccatgcccaggttaacaaagaactgtgatatatagagtgtctaattacaaaatcatatacgatttatttaattctcttctgtattgtaacttagatgattcccaaggactctaataaaaaatcacttcattgtatttggaaacaaaaacatcattcattaattacttattttctttccataggttttaatattttgagagtgtcttttttatttcattcatgaacttttgtatttttcatttttcatttgatttgtaaatttacttatgttaaaaataaaccatttattttcagctttg

SEQ ID NO: 25 - protein, 570 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKVPAPRYTVELACGFGATVLLVVILIVVYHVYWLEMVLFYRAHFGTDETILDGKEYDIYVSYARNAEEEEFVLLTLRGVLENEFGYKLCIFDRDSLPGGIVTDETLSFIQKSRRLLVVLSPNYVLQGTQALLELKAGLENMASRGNINVILVQYKAVKETKVKELKRAKTVLTVIKWKGEKSKYPQGRFWKQLQVAMPVKKSPRRSSSDEQGLSYSSLKNV

SEQ ID NO: 26 - DNA, 2063 bp, Homo sapiens
tgccgggatccaggtctccggggtccgctttggccagaggcgcggaaggaagcagtgcccggcgacactgcacccatcccggctgcttttgctgcgccctctcagcttcccaagaaaggcatcgtcatgtgatcatcacctaagaactagaacatcagcaggccctagaagcctcactcttgcccctccctttaatatctcaaaggatgacacttctgtggtgtgtagtgagtctctacttttatggaatcctgcaaagtgatgcctcagaacgctgcgatgactggggactagacaccatgaggcaaatccaagtgtttgaagatgagccagctcgcatcaagtgcccactctttgaacacttcttgaaattcaactacagcacagcccattcagctggccttactctgatctggtattggactaggcaggaccgggaccttgaggagccaattaacttccgcctccccgagaaccgcattagtaaggagaaagatgtgctgtggttccggcccactctcctcaatgacactggcaactatacctgcatgttaaggaacactacatattgcagcaaagttgcatttcccttggaagttgttcaaaaagacagctgtttcaattcccccatgaaactcccagtgcataaactgtatatagaatatggcattcagaggatcacttgtccaaatgtagatggatattttccttccagtgtcaaaccgactatcacttggtatatgggctgttataaaatacagaattttaataatgtaatacccgaaggtatgaacttgagtttcctcattgccttaatttcaaataatggaaattacacatgtgttgttacatatccagaaaatggacgtacgtttcatctcaccaggactctgactgtaaaggtagtaggctctccaaaaaatgcagtgccccctgtgatccattcacctaatgatcatgtggtctatgagaaagaaccaggagaggagctactcattccctgtacggtctattttagttttctgatggattctcgcaatgaggtttggtggaccattgatggaaaaaaacctgatgacatcactattgatgtcaccattaacgaaagtataagtcatagtagaacagaagatgaaacaagaactcagattttgagcatcaagaaagttacctctgaggatctcaagcgcagctatgtctgtcatgctagaagtgccaaaggcgaagttgccaaagcagccaaggtgaagcagaaaggtaatagatgcggtcagtgatgaatctctcagctccaaattaacattgtggtgaataaggacaaaaggagagattgagaacaagagagctccagcacctagcccgacggcatctaacccatagtaatgaatcaaacttaaatgaaaaatatgaaagttttcatctatgtaagatactcaaaatattgtttctgatattgttagtaccgtaatgcccaaatgtagctaaaaaaatcgacgtgagtacagtgagacacaattttgtgtctgtacaattatgaaaaattaaaaacaaagaaaatattcaaagctaccaaagatagaaaaaactggtagagccacatattgttggtgaattattaagacccttttaaaaatcattcatggtagagtttaagagtcataaaaaagattgcatcatctgacctaagactttcggaatttttcctgaacaaataacagaaagggaattatataccttttaatattattagaagcattatctgtagttgtaaaacattattaatagcagccatccaattgtatgcaactaattaaggtattgaatgtttattttccaaaaatgcataattataatattattttaaacactatgtatcaatatttaagcaggtttataatataccagcagccacaattgctaaaatgaaaatcatttaaattatgattttaaatggtataaacatgatttctatgttgatagtactatattattctacaataaatggaaattataaagccttcttgtcagaagtgctgctcctaaaaaaaaaaaaaaaaaaaaaa

SEQ ID NO: 27 - protein, 356 aa, Homo sapiens
MTLLWCVVSLYFYGILQSDASERCDDWGLDTMRQIQVFEDEPARIKCPLFEHFLKFNYSTAHSAGLTLIWYWTRQDRDLEEPINFRLPENRISKEKDVLWFRPTLLNDTGNYTCMLRNTTYCSKVAFPLEVVQKDSCFNSPMKLPVHKLYIEYGIQRITCPNVDGYFPSSVKPTITWYMGCYKIQNFNNVIPEGMNLSFLIALISNNGNYTCVVTYPENGRTFHLTRTLTVKVVGSPKNAVPPVIHSPNDHVVYEKEPGEELLIPCTVYFSFLMDSRNEVWWTIDGKKPDDITIDVTINESISHSRTEDETRTQILSIKKVTSEDLKRSYVCHARSAKGEVAKAAKVKQKGNRCGQ

SEQ ID NO: 28 - DNA, 3589 bp, Homo sapiens
cgcggacccggccggcccaggcccgcgcccgccgcggccctgagaggccccggcaggtcccggcccggcggcggcagccatggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctgatgtgttcacctgggcagatcccccaaatccggaagtcaaagttctcatggtcagaagttctcatggtgcacgagtcctcagcactctgccggcagtgggggtgggggcccatgcccgcgggggagagaaggaggtggccctgctgttctaggctctgtgggcataggcaggcagagtggaaccctgcctccatgccagcatctgggggcaaggaaggctggcatcatccagtgaggaggctggcgcatgttgggaggctgctggctgcacagacccgtgaggggaggagaggggctgctgtgcaggggtgtggagtagggagctggctcccctgagagccatgcagggcgtctgcagcccaggcctctggcagcagctctttgcccatctctttggacagtggccaccctgcacaatggggccgacgaggcctagggccctcctacctgcttacaatttggaaaagtgtggccgggtgcggtggctcacgcctgtaatcccagcactttgggaggccaaggcaggaggatcgctggagcccagtaggtcaagaccagccagggcaacatgatgagaccctgtctctgccaaaaaattttttaaactattagcctggcgtggtagcgcacgcctgtggtcccagctgctggggaggctgaagtaggaggatcatttatgcttgggaggtcgaggctgcagtgagtcatgattgtatgactgcactccagcctgggtgacagagcaagaccctgtttcaaaaagaaaaaccctgggaaaagtgaagtatggctgtaagtctcatggttcagtcctagcaagaagcgagaattctgagatcctccagaaagtcgagcagcacccacctccaacctcgggccagtgtcttcaggctttactggggacctgcgagctggcctaatgtggtggcctgcaagccaggccatccctgggcgccacagacgagctccgagccaggtcaggcttcggaggccacaagctcagcctcaggcccaggcactgattgtggcagaggggccactacccaaggtctagctaggcccaagacctagttacccagacagtgagaagcccctggaaggcagaaaagttgggagcatggcagacagggaagggaaacattttcagggaaaagacatgtatcacatgtcttcagaagcaagtcaggtttcatgtaaccgagtgtcctcttgcgtgtccaaaagtagcccagggctgtagcacaggcttcacagtgattttgtgttcagccgtgagtcacactacatgcccccgtgaagctgggcattggtgacgtccaggttgtccttgagtaataaaaacgtatgttgcaataaaaaaaaaaaaaaaaaa

SEQ ID NO: 29 - protein, 712 aa, Homo sapiens
MAGGPGPGEPAAPGAQHFLYEVPPWVMCRFYKVMDALEPADWCQFAALIVRDQTELRLCERSGQRTASVLWPWINRNARVADLVHILTHLQLLRARDIITAWHPPAPLPSPGTTAPRPSSIPAPAEAEAWSPRKLPSSASTFLSPAFPGSQTHSGPELGLVPSPASLWPPPPSPAPSSTKPGPESSVSLLQGARPFPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLKDLVEEEAEEAGVALRSTQSTLQAGLAADAWAAPIAMQIYKKHLDPRPGPCPPELGLGLGQLACCCLHRRAKRRPPMTQVYERLEKLQAVVAGVPGHSEAASCIPPSPQENSYVSSTGRAHSGAAPWQPLAAPSGASAQAAEQLQRGPNQPVESDESLGGLSAALRSWHLTPSCPLDPAPLREAGCPQGDTAGESSWGSGPGSRPTAVEGLALGSSASSSSEPPQIIINPARQKMVQKLALYEDGALDSLQLLSSSSLPGLGLEQDRQGPEESDEFQS

SEQ ID NO: 30 - DNA, 3499 bp, Homo sapiens
cgcggacccggccggcccaggcccgcgcccgccgcggccctgagaggccccggcaggtcccggcccggcggcggcagccatggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctgaaagacctggtggaagaggaggctgaggaggctggagtggctttgagaagcacccagagcacactgcaagcaggtctggctgcagatgcctgggctgctcccatcgccatgcagatctacaagaagcacctggaccccaggcccgggccctgcccacctgagctgggcctgggcctgggccagctggcctgctgctgcctgcaccgccgggccaaaaggaggcctcctatgacccaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctgatgtgttcacctgggcagatcccccaaatccggaagtcaaagttctcatggtcagaagttctcatggtgcacgagtcctcagcactctgccggcagtgggggtgggggcccatgcccgcgggggagagaaggaggtggccctgctgttctaggctctgtgggcataggcaggcagagtggaaccctgcctccatgccagcatctgggggcaaggaaggctggcatcatccagtgaggaggctggcgcatgttgggaggctgctggctgcacagacccgtgaggggaggagaggggctgctgtgcaggggtgtggagtagggagctggctcccctgagagccatgcagggcgtctgcagcccaggcctctggcagcagctctttgcccatctctttggacagtggccaccctgcacaatggggccgacgaggcctagggccctcctacctgcttacaatttggaaaagtgtggccgggtgcggtggctcacgcctgtaatcccagcactttgggaggccaaggcaggaggatcgctggagcccagtaggtcaagaccagccagggcaacatgatgagaccctgtctctgccaaaaaattttttaaactattagcctggcgtggtagcgcacgcctgtggtcccagctgctggggaggctgaagtaggaggatcatttatgcttgggaggtcgaggctgcagtgagtcatgattgtatgactgcactccagcctgggtgacagagcaagaccctgtttcaaaaagaaaaaccctgggaaaagtgaagtatggctgtaagtctcatggttcagtcctagcaagaagcgagaattctgagatcctccagaaagtcgagcagcacccacctccaacctcgggccagtgtcttcaggctttactggggacctgcgagctggcctaatgtggtggcctgcaagccaggccatccctgggcgccacagacgagctccgagccaggtcaggcttcggaggccacaagctcagcctcaggcccaggcactgattgtggcagaggggccactacccaaggtctagctaggcccaagacctagttacccagacagtgagaagcccctggaaggcagaaaagttgggagcatggcagacagggaagggaaacattttcagggaaaagacatgtatcacatgtcttcagaagcaagtcaggtttcatgtaaccgagtgtcctcttgcgtgtccaaaagtagcccagggctgtagcacaggcttcacagtgattttgtgttcagccgtgagtcacactacatgcccccgtgaagctgggcattggtgacgtccaggttgtccttgagtaataaaaacgtatgttgcaataaaaaaaaaaaaaaaaaa

SEQ ID NO: 31 - protein, 682 aa, Homo sapiens
MAGGPGPGEPAAPGAQHFLYEVPPWVMCRFYKVMDALEPADWCQFAALIVRDQTELRLCERSGQRTASVLWPWINRNARVADLVHILTHLQLLRARDIITAWHPPAPLPSPGTTAPRPSSIPAPAEAEAWSPRKLPSSASTFLSPAFPGSQTHSGPELGLVPSPASLWPPPPSPAPSSTKPGPESSVSLLQGARPFPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLKDLVEEEAEEAGVALRSTQSTLQAGLAADAWAAPIAMQIYKKHLDPRPGPCPPELGLGLGQLACCCLHRRAKRRPPMTQENSYVSSTGRAHSGAAPWQPLAAPSGASAQAAEQLQRGPNQPVESDESLGGLSAALRSWHLTPSCPLDPAPLREAGCPQGDTAGESSWGSGPGSRPTAVEGLALGSSASSSSEPPQIIINPARQKMVQKLALYEDGALDSLQLLSSSSLPGLGLEQDRQGPEESDEFQS

SEQ ID NO: 32 - DNA, 3352 bp, Homo sapiens
cgcggacccggccggcccaggcccgcgcccgccgcggccctgagaggccccggcaggtcccggcccggcggcggcagccatggccggggggccgggcccgggggagcccgcagcccccggcgcccagcacttcttgtacgaggtgccgccctgggtcatgtgccgcttctacaaagtgatggacgccctggagcccgccgactggtgccagttcgccgccctgatcgtgcgcgaccagaccgagctgcggctgtgcgagcgctccgggcagcgcacggccagcgtcctgtggccctggatcaaccgcaacgcccgtgtggccgacctcgtgcacatcctcacgcacctgcagctgctccgtgcgcgggacatcatcacagcctggcaccctcccgccccgcttccgtccccaggcaccactgccccgaggcccagcagcatccctgcacccgccgaggccgaggcctggagcccccggaagttgccatcctcagcctccaccttcctctccccagcttttccaggctcccagacccattcagggcctgagctcggcctggtcccaagccctgcttccctgtggcctccaccgccatctccagccccttcttctaccaagccaggcccagagagctcagtgtccctcctgcagggagcccgcccctttccgttttgctggcccctctgtgagatttcccggggcacccacaacttctcggaggagctcaagatcggggagggtggctttgggtgcgtgtaccgggcggtgatgaggaacacggtgtatgctgtgaagaggctgaaggagaacgctgacctggagtggactgcagtgaagcagagcttcctgaccgaggtggagcagctgtccaggtttcgtcacccaaacattgtggactttgctggctactgtgctcagaacggcttctactgcctggtgtacggcttcctgcccaacggctccctggaggaccgtctccactgccagacccaggcctgcccacctctctcctggcctcagcgactggacatccttctgggtacagcccgggcaattcagtttctacatcaggacagccccagcctcatccatggagacatcaagagttccaacgtccttctggatgagaggctgacacccaagctgggagactttggcctggcccggttcagccgctttgccgggtccagccccagccagagcagcatggtggcccggacacagacagtgcggggcaccctggcctacctgcccgaggagtacatcaagacgggaaggctggctgtggacacggacaccttcagctttggggtggtagtgctagagaccttggctggtcagagggctgtgaagacgcacggtgccaggaccaagtatctggtgtacgagaggctagagaagctgcaggcagtggtggcgggggtgcccgggcattcggaggccgccagctgcatccccccttccccgcaggagaactcctacgtgtccagcactggcagagcccacagtggggctgctccatggcagcccctggcagcgccatcaggagccagtgcccaggcagcagagcagctgcagagaggccccaaccagcccgtggagagtgacgagagcctaggcggcctctctgctgccctgcgctcctggcacttgactccaagctgccctctggacccagcacccctcagggaggccggctgtcctcagggggacacggcaggagaatcgagctgggggagtggcccaggatcccggcccacagccgtggaaggactggcccttggcagctctgcatcatcgtcgtcagagccaccgcagattatcatcaaccctgcccgacagaagatggtccagaagctggccctgtacgaggatggggccctggacagcctgcagctgctgtcgtccagctccctcccaggcttgggcctggaacaggacaggcaggggcccgaagaaagtgatgaatttcagagctgatgtgttcacctgggcagatcccccaaatccggaagtcaaagttctcatggtcagaagttctcatggtgcacgagtcctcagcactctgccggcagtgggggtgggggcccatgcccgcgggggagagaaggaggtggccctgctgttctaggctctgtgggcataggcaggcagagtggaaccctgcctccatgccagcatctgggggcaaggaaggctggcatcatccagtgaggaggctggcgcatgttgggaggctgctggctgcacagacccgtgaggggaggagaggggctgctgtgcaggggtgtggagtagggagctggctcccctgagagccatgcagggcgtctgcagcccaggcctctggcagcagctctttgcccatctctttggacagtggccaccctgcacaatggggccgacgaggcctagggccctcctacctgcttacaatttggaaaagtgtggccgggtgcggtggctcacgcctgtaatcccagcactttgggaggccaaggcaggaggatcgctggagcccagtaggtcaagaccagccagggcaacatgatgagaccctgtctctgccaaaaaattttttaaactattagcctggcgtggtagcgcacgcctgtggtcccagctgctggggaggctgaagtaggaggatcatttatgcttgggaggtcgaggctgcagtgagtcatgattgtatgactgcactccagcctgggtgacagagcaagaccctgtttcaaaaagaaaaaccctgggaaaagtgaagtatggctgtaagtctcatggttcagtcctagcaagaagcgagaattctgagatcctccagaaagtcgagcagcacccacctccaacctcgggccagtgtcttcaggctttactggggacctgcgagctggcctaatgtggtggcctgcaagccaggccatccctgggcgccacagacgagctccgagccaggtcaggcttcggaggccacaagctcagcctcaggcccaggcactgattgtggcagaggggccactacccaaggtctagctaggcccaagacctagttacccagacagtgagaagcccctggaaggcagaaaagttgggagcatggcagacagggaagggaaacattttcagggaaaagacatgtatcacatgtcttcagaagcaagtcaggtttcatgtaaccgagtgtcctcttgcgtgtccaaaagtagcccagggctgtagcacaggcttcacagtgattttgtgttcagccgtgagtcacactacatgcccccgtgaagctgggcattggtgacgtccaggttgtccttgagtaataaaaacgtatgttgcaataaaaaaaaaaaaaaaaaa

SEQ ID NO: 33 - protein, 633 aa, Homo sapiens
MAGGPGPGEPAAPGAQHFLYEVPPWVMCRFYKVMDALEPADWCQFAALIVRDQTELRLCERSGQRTASVLWPWINRNARVADLVHILTHLQLLRARDIITAWHPPAPLPSPGTTAPRPSSIPAPAEAEAWSPRKLPSSASTFLSPAFPGSQTHSGPELGLVPSPASLWPPPPSPAPSSTKPGPESSVSLLQGARPFPFCWPLCEISRGTHNFSEELKIGEGGFGCVYRAVMRNTVYAVKRLKENADLEWTAVKQSFLTEVEQLSRFRHPNIVDFAGYCAQNGFYCLVYGFLPNGSLEDRLHCQTQACPPLSWPQRLDILLGTARAIQFLHQDSPSLIHGDIKSSNVLLDERLTPKLGDFGLARFSRFAGSSPSQSSMVARTQTVRGTLAYLPEEYIKTGRLAVDTDTFSFGVVVLETLAGQRAVKTHGARTKYLVYERLEKLQAVVAGVPGHSEAASCIPPSPQENSYVSSTGRAHSGAAPWQPLAAPSGASAQAAEQLQRGPNQPVESDESLGGLSAALRSWHLTPSCPLDPAPLREAGCPQGDTAGESSWGSGPGSRPTAVEGLALGSSASSSSEPPQIIINPARQKMVQKLALYEDGALDSLQLLSSSSLPGLGLEQDRQGPEESDEFQS

SEQ ID NO: 34 - RNA, 23 nt, Artificial sequence
  IL-1 alpha miRNA
uguaaacauccuacacucucagc

SEQ ID NO: 35 - RNA, 21 nt, Artificial sequence
  IL-1 alpha miRNA
uguaaacauccuacacucagc

SEQ ID NO: 36 - RNA, 23 nt, Artificial sequence
  IL-1 alpha miRNA
uguaaacauccucgacuggaagc

SEQ ID NO: 37 - RNA, 22 nt, Artificial sequence
  IL-1 alpha miRNA
uggcucaguucagcaggaacag

SEQ ID NO: 38 - RNA, 22 nt, Artificial sequence
  IL-1R1 miRNA
uauggcuuuucauuccuaugug

SEQ ID NO: 39 - RNA, 20 nt, Artificial sequence
  IL-1R1 miRNA
ccucugggcccuuccuccag

SEQ ID NO: 40 - RNA, 22 nt, Artificial sequence
  IL-1R1 miRNA
uggacggagaacugauaagggu

SEQ ID NO: 41 - RNA, 21 nt, Artificial sequence
  IL-1R1 miRNA
acagcaggcacagacaggcag

SEQ ID NO: 42 - RNA, 22 nt, Artificial sequence
  IL-1R1 miRNA
gugaaauguuuaggaccacuag

SEQ ID NO: 43 - RNA, 21 nt, Artificial sequence
  IL-1R1 miRNA
gccccugggccuauccuagaa

SEQ ID NO: 44 - RNA, 22 nt, Artificial sequence
  IL-1R1 miRNA
uccuucauuccaccggagucug

SEQ ID NO: 45 - protein, 15 aa, Homo sapiens
  MISC_FEATURE (10)..(10): Wherein Xaa is 2-azetidine-1-carboxylic acid.
FEWTPGWYQXYALPL